(19) United States
(12) Patent Application Publication
(10) Pub. No.: US 2021/0393523 A1
(43) Pub. Date: Dec. 23, 2021

(54) AROMATIC RING SUBSTITUTED AMPHIPHILIC POLYMERS AS DRUG DELIVERY SYSTEMS

(71) Applicants: Avldea Technologies, Inc., Baltimore, MD (US); The United States of America, as represented by the Secretary, Department of Health and Human Servic, Bethesda, MD (US)

(72) Inventors: Geoffrey Martin Lynn, Baltimore, MD (US); Yaling Zhu, Gaithersburg, MD (US); Sarah R. Nichols, Baltimore, MD (US); Andrew Scott lshizuka, Washington, DC (US)

(73) Assignees: Avldea Technologies, Inc., Baltimore, MD (US); The United States of America, as represented by the Secretary, Department of Health and Human Servic, Bethesda, MD (US)

(21) Appl. No.: 17/282,447

(22) PCT Filed: Oct. 2, 2019
(86) PCT No.: PCT/US19/54343

§ 371 Date: Apr. 2, 2021

Related U.S. Application Data
(60) Provisional application No. 62/851,523, filed on May 22, 2019.
(60) Provisional application No. 62/740,837, filed on Oct. 3, 2018.

(51) Int. Cl. A61K 9/107; A61K 9/127; A61K 45/06; A61K 47/30; A61K 47/56; A61K 47/69

(52) U.S. Cl. CPC A61K 9/1075; A61K 9/1273; A61K 45/06; B82Y 5/00; A61K 47/56; A61K 47/6915; A61K 47/6907; A61K 47/30

(57) ABSTRACT

An amphiphilic block copolymer having any one of the formulas S-[B]-H, S-[B]-H(D), D-[B]-H, S-B(D)-H, S-[B]-H-[B]-S, S-[B]-H(D)-[B]-S, D-[B]-H-[B]-S, D-[B]-H-[B]-D, S-B(D)-H-[B]-S or S-B(D)-H-B(D)-S; wherein S is a hydrophilic surface stabilizing group; B is a spacer group; H is a hydrophobic polymer or oligomer; D is a drug molecule; ( ) denotes that the group is bonded directly or indirectly as a side chain or as part of a side chain group to the adjacent group; [ ] denotes that the group is optional; and - denotes that each of the adjacent S, B, H or D are linked directly to one another or indirectly to one another via a linker group.

### PRIORITY

The present application claims priority from U.S. Provisional Patent Application No. 62/740,837 filed on Oct. 3, 2018 and U.S. Provisional Patent Application No. 62/851,523 filed on May 22, 2019, which are hereby incorporated by reference in their entirety.

This invention was created in the performance of a Cooperative Research and Development Agreement with the National Institutes of Health, an Agency of the Department of Health and Human Services. The Government of the United States has certain rights in this invention.

### FIELD OF DISCLOSURE

The present disclosure relates to novel polymer compositions that can be used to form micelle structures or polymersomes, methods of manufacturing the polymer compositions, processes for formulating drug molecules with the polymer compositions that form micelles or polymersomes, and therapeutic uses of the micelles and polymersomes for drug delivery.

### BACKGROUND

Many drugs used for the diagnosis, treatment, cure or prevention of disease are limited by poor water solubility and/or suboptimal pharmacokinetics (PK), which may be improved through the use of drug delivery systems.

Drug delivery systems are broadly inclusive of any materials that impact the solubility, pharmacokinetics and/or or cellular and subcellular distribution of a drug to impact its therapeutic effect in the body. Some known drug delivery systems include particle emulsions, lipid-based micelles and multi-lamellar vesicles (e.g., liposomes), polymer-based micelles and vesicles (e.g., polymersomes), mineral salts and drug conjugates of macromolecules and small molecules (e.g., albumin binding molecules).

Drug delivery systems have been used for a broad range of different applications and therapeutic indications. Examples of drug delivery systems used for cancer treatment include the use of liposomal particles for encapsulating hydrophobic chemotherapeutic molecules, as well as hydrophilic macromolecules as carriers of anthracyclines, taxanes (e.g., paclitaxel) and platinum-based anti-neoplastic drugs. Moreover, various types of drug delivery systems, including mineral salts (e.g., alum), liposomes and emulsions have been used in the delivery of antigens and immuno-stimulants (“adjuvants”) in vaccines for cancer and infectious diseases. Drug delivery systems have also been used for modulating the PK of recombinant proteins and for targeting diagnostic or combined therapeutic and diagnostic agents (“theranostics”) to specific tissues or anatomical sites.

Despite the myriad benefits, poor chemical definition and reproducibility of formulation characteristics limit the potential clinical translatability of most drug delivery systems. For instance, many particle emulsion technologies based on PLGA and liposomes rely on an empirical drug loading process that often results in low and variable drug loading. An additional challenge is that the process for forming particles with commonly used lipid and polymer-based systems typically results in heterogeneous mixtures of particles with a broad distribution of sizes. While processes can be optimized to ensure particle size distribution reproducibility, sterile-filtering such mixtures can still represent a major challenge as particles at or above the size cut-off for sterile filtration membranes (e.g., >200 nm) can clog membranes and complicate manufacturing.

Drug delivery systems based on micelle-forming amphiphilic molecules offer the potential advantages over other drug delivery technologies that micelles can have narrow distribution of particle sizes that are often well below the cut-off for membrane pore size for sterile filters. This provides the benefits that the particles are better defined and can be easily sterile-filtered without membrane clogging.

Drug delivery systems using micelle-forming amphiphilic molecules typically use hydrophobic molecules based on fatty acids (FAs), lipids, cholesterol or hydrophobic polymers, such as PLGA, polystyrene, poly[2-(2-methoxyethoxy)ethyl methacrylate] (or “DEGMA”) and lauryl methacrylate (LMA). While these systems have been used with varying degrees of success, often in preclinical models, poor solubility in water miscible solvents and relatively high critical micellar concentrations, which causes the micelles to fall apart rapidly when used in vivo, are two major challenges to the adoption of micelle-based drug delivery systems for clinical uses.

Additional challenges often associated with the use of micelle-forming amphiphilic molecules for drug delivery systems are poor chemical definition (e.g., broad molecular weight distribution) of many of the polymer systems used; particle size variability; and often low and variable drug molecule loading.

There is a need for drug delivery systems that overcome or address one or more of the limitations of known drug delivery systems.

### SUMMARY

Disclosed herein are compositions and methods of manufacturing micelle- and polymersome-forming amphiphilic copolymers that are chemically defined, soluble in water miscible solvents and/or are amenable to chemical conjugation for precise drug loading thereby ameliorating many limitations of contemporary drug delivery systems. In certain embodiments, such amphiphilic polymers are based on linear block (e.g., diblock and triblock) and brush copolymer architectures.

According to a first aspect of the present disclosure, there is provided an amphiphilic block copolymer having any one of the formulas, S-[B]-H, S-[B]-H(D), D-[B]-H, S-B(D)-H, S-[B]-H-[B]-S, S-[B]-H(D)-[B]-S, D-[B]-H-[B]-S, D-[B]-H-[B]-D, S-B(D)-H-[B]-S or S-B(D)-H-B(D)-S; wherein S is a hydrophilic surface stabilizing group; B is a spacer group; H is a hydrophobic polymer or oligomer; D is a drug molecule; ( ) denotes that the group is bonded directly or indirectly as a side chain or as part of a side chain group to the adjacent group; [ ] denotes that the group is optional; and - denotes that each of the adjacent S, B, H or D are linked directly to one another or indirectly to one another via a linker group.

In certain embodiments, the hydrophobic polymer or oligomer comprises three or more side chain aromatic groups. In certain of these embodiments, the hydrophobic polymer or oligomer comprises three or more side chain aromatic amine groups.

In certain embodiments, the hydrophobic polymer or oligomer is a poly(amino acid) comprising from about 3 to about 80 monomers, such as from about 3 to about 30 aromatic amino acids.

In certain embodiments, the hydrophobic polymer or oligomer comprises a poly(amino acid)-based polymer comprised of hydrophobic monomers ( [symbol] ), spacer monomers (m), charged amino acid monomers (n) for charge compensation, and functional group containing monomers (o) for drug molecule (D) attachment.

In certain embodiments, the hydrophilic surface stabilizing group comprises one or more charged functional groups.

In certain embodiments, the surface stabilizing group provides a high net charge (>+4, or <−4).

The hydrophilic surface stabilizing group may comprise one or more mono-saccharide or oligo-saccharide molecules.

According to a second aspect of the present disclosure, there is provided a composition comprising the amphiphilic block copolymer of the first aspect.

According to a third aspect of the present disclosure, there is provided a particle comprising the amphiphilic block copolymer of the first aspect.

According to a fourth aspect of the present disclosure, there is provided a polymersome particle comprising the amphiphilic block copolymer of the first aspect.

According to a fifth aspect of the present disclosure, there is provided a micelle particle comprising the amphiphilic block copolymer of the first aspect.

According to a sixth aspect of the present disclosure, there is provided use of the amphiphilic block copolymer of the first aspect to form a particle.

According to a seventh aspect of the present disclosure, there is provided use of the amphiphilic block copolymer of the first aspect to form a polymersome particle.

According to an eighth aspect of the present disclosure, there is provided use of the amphiphilic block copolymer of the first aspect to form a micelle particle.

According to a ninth aspect of the present disclosure, there is provided a mosaic particle comprising two or more different amphiphilic block copolymers selected from any one of the formulas, S-[B]-H, S-[B]-H(D), D-[B]-H, S-B(D)-H, S-[B]-H-[B]-S, S-[B]-H(D)-[B]-S, D-[B]-H-[B]-S, D-[B]-H-[B]-D, S-B(D)-H-[B]-S or S-B(D)-H-B(D)-S; wherein S is a hydrophilic surface stabilizing group; B is a spacer group; H is a hydrophobic polymer or oligomer; D is a drug molecule; ( ) denotes that the group is bonded directly or indirectly as a side chain or as part of a side chain group to the adjacent group; [ ] denotes that the group is optional; and - denotes that each of the adjacent S, B, H or D are linked directly to one another or indirectly to one another via a linker group.

According to a tenth aspect of the present disclosure, there is provided a particle of any one of the third, fourth, fifth or ninth aspects further comprising a drug molecule conjugated to a hydrophobic polymer or oligomer, i.e. D-H.

According to an eleventh aspect of the present disclosure, there is provided a method of preparing particles comprising an amphiphilic block copolymer membrane and at least one drug molecule (D) encapsulated therein, said method comprising:

providing an amphiphilic block copolymer having any one of the formulas, S-[B]-H, S-[B]-H(D), D-[B]-H, S-B(D)-H, S-[B]-H-[B]-S, S-[B]-H(D)-[B]-S, D-[B]-H-[B]-S, D-[B]-H-[B]-D, S-B(D)-H-[B]-S or S-B(D)-H-B(D)-S; wherein S is a hydrophilic surface stabilizing group; B is a spacer group; H is a hydrophobic polymer or oligomer; D is a drug molecule; ( ) denotes that the group is bonded directly or indirectly as a side chain or as part of a side chain group to the adjacent group; [ ] denotes that the group is optional; and - denotes that each of the adjacent S, B, H or D are linked directly to one another or indirectly to one another via a linker group; and preparing an aqueous solution comprising said amphiphilic block copolymer under conditions to produce particles having the at least one drug molecule encapsulated therein.

### BRIEF DESCRIPTION OF FIGURES

Embodiments of the present disclosure will be discussed with reference to the accompanying figures wherein:

FIG. 1 is a schematic representation of two different embodiments of nanoparticle micelle carriers of drug molecules (D) comprised of amphiphilic block copolymers and drug molecules described in the present disclosure, wherein (a) the drug molecule is either linked directly to the amphiphilic block copolymer (e.g., S-B-H(D)) or (b) is admixed with the amphiphilic block copolymer (e.g., S-B-H+D) and incorporates within the micelle; the examples provided in FIG. 1 are not meant to be limiting;

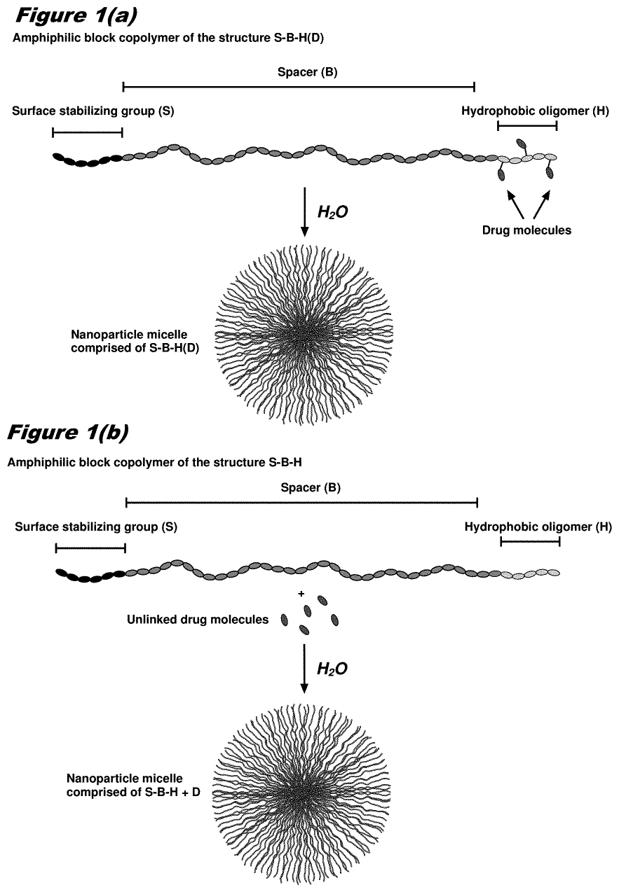

FIG. 2 shows both the relationship between (a) net charge and particle size of amphiphilic block copolymers and (b) the relationship between S-B architecture and the propensity of amphiphilic block copolymers with different size poly(amino acid)-based hydrophobic polymer or oligomers (H) to aggregate (i.e. OD at 490 nm>0.05); in figure panel (a), the number of charged functional groups comprising the charged molecule (C)-based surface stabilizing group (S) is varied to provide poly(amino acid)-based amphiphilic block copolymers, wherein the hydrophobic polymer or oligomer is comprised of between 5 to 20 para-aminophenylalanine monomers, with net charge between +2 and +8; the data show that increasing net charge increases the propensity of the amphiphilic block copolymers to form nanoparticle micelles (<100 nm, diameter); in figure panel (b) the number of charged functional groups comprising the charged molecule (C)-based surface stabilizing group (S) is kept constant (+4 net charge) but the architecture of the poly(amino acid)-based amphiphilic block copolymers is varied, wherein the hydrophobic polymer or oligomer is comprised of between 5 to 20 para-aminophenylalanine monomers (see figure key; for example, (10)2 means the poly(amino acid)-based hydrophobic polymer or oligomer (H) has two branches and there are two linear para-aminophenylalanine oligopeptides comprised of 10 monomer units linked to each of the branches); the data show that amphiphilic block copolymers with brush S-B architecture show lower propensity to aggregate than those with linear or cone architecture;

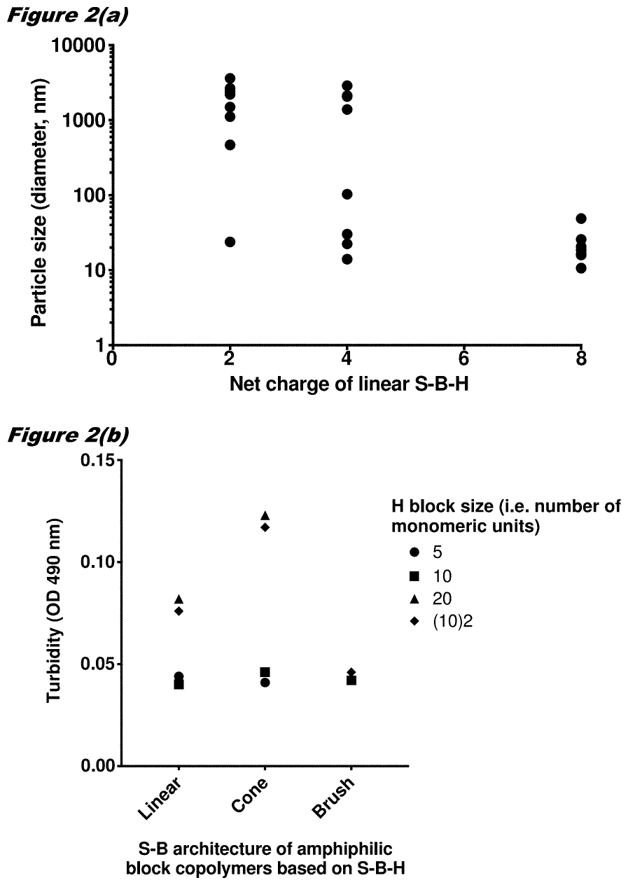

FIG. 3 shows that nanoparticles comprised of amphiphilic block copolymers that comprise CpG-Ahx-W5 (i.e. CpG oligonucleotide linked to DBCO-Ahx-Trp-Trp-Trp-Trp-Trp through an azide-DBCO linkage, as described under example 13) and Compound 33, an imidazoquinoline-based TLR-7/8a drug (D), referred to as “2BXy,” promote regression of established tumors;

FIG. 4 shows that nanoparticles comprised of amphiphilic block copolymers with the formula S-[B]-H that are associated with 2BXy (i.e. S-[B]-H+D) or are linked covalently to 2BXy (i.e. S-[B]-H(D) promote regression of established tumors; K₈—PEG₂₄-W₅ is Compound 88, K₈—PEG₂₄-F′₂₀ is Compound 82, K₇—SG₁₂-F′₂₀ is Compound 84, HPMA-F′₂₀ is Azide-pHPMA linked to DBCO-Ahx-(F′)₁₀ through an azide-DBCO linkage (as described under example 13) and [OH-PEG₂₄]₄-2BXy4 is Compound 78; note: Compound 78 is an amphiphilic block copolymer that has brush S-B architecture and neutral surface charge with a branched poly(amino acid)-based hydrophobic polymer or oligomer (H) that is covalently attached to four 2BXy drug molecules (D); and,

FIG. 5 shows that nanoparticles comprised of amphiphilic block copolymers with the formula S-[B]-H that associated with a first drug molecule, 2BXy, and a second drug, Doxorubicin, (i.e. S-[B]-H+(1)D+(2)D) promote regression of established tumors; [OH-PEG₂₄]₄-F′₁₀ is Compound 80, which has a brush S-B architecture with neutral surface charge and a linear poly(amino acid)-based hydrophobic polymer or oligomer (H).

### DETAILED DESCRIPTION

The present disclosure may be understood more readily by reference to the following description taken in connection with the accompanying figures and/or examples, which form a part of this disclosure. It is to be understood that the various embodiments are not limited to the specific compositions, devices, methods, applications, conditions or parameters described and/or shown herein, and that the terminology used herein is for the purpose of describing particular embodiments by way of example only and is not intended to be limiting.

It is to be appreciated that certain features that are, for clarity, described herein in the context of separate embodiments, may also be provided in combination in a single embodiment. Conversely, various features that are, for brevity, described in the context of a single embodiment, may also be provided separately or in any sub-combination.

As used in this specification and the appended claims, the singular forms “a,” “an,” and “the” include plural referents unless the content clearly dictates otherwise.

When a range of values is expressed herein, another embodiment includes from the one particular value and/or to the other particular value. Similarly, when values are expressed as approximations, by use of the antecedent “about,” it will be understood that the particular value forms another embodiment. All ranges are inclusive and combinable. Furthermore, references to values stated in ranges include each and every value within that range.

Details of terms and methods are given below to provide greater clarity concerning compounds, compositions, methods and the use(s) thereof for the purpose of guiding those of ordinary skill in the art in the practice of the present disclosure. The terminology in this disclosure is understood to be useful for the purpose of providing a better description of particular embodiments and should not be considered limiting.

About: In the context of the present disclosure, “about” when referring to a measurable value such as an amount, a temporal duration, and the like, is meant to encompass variations of ±20%, ±10%, ±5%, 1%, or ±0.1% from the specified value, as such variations are appropriate to perform the disclosed methods. For example, “about 10” refers to 9.5 to 10.5. A ratio of “about 5:1” refers to a ratio from 4.75:1 to 5.25:1.

Administration: To provide or give to a subject an agent, for example, an immunogenic composition comprising amphiphilic block copolymers and drug(s) as described herein, by any effective route. Exemplary routes of administration include, but are not limited to, oral, injection (such as subcutaneous, intramuscular, intradermal, intraperitoneal, and intravenous), transdermal (for example, topical), intranasal, vaginal, and inhalation routes.

“Administration of” and “administering a” compound should be understood to mean providing a compound, a prodrug of a compound, or a pharmaceutical composition as described herein. The compound or composition can be administered by another person to the subject or it can be self-administered by the subject.

Amphiphilic: The term “amphiphilic” is used herein to mean a substance containing both hydrophilic or polar (water-soluble) and hydrophobic (water-insoluble) groups.

Aromatic, aryl or Ar: Aromatic compounds are unsaturated cyclic rings with an odd number of pairs of pi orbital electrons that are delocalized between the carbon or nitrogen atoms forming the ring. Aromatic compounds comprise six to ten ring atoms (e.g., C6-C10 aromatic or C6-C10 aryl) which have at least one ring having a conjugated pi electron system which is carbocyclic (e.g., phenyl, fluorenyl, and naphthyl) or heterocyclic. Whenever it appears herein, a numerical range such as “6 to 10” refers to each integer in the given range; e.g., “6 to 10 ring atoms” means that the aryl group may consist of 6 ring atoms, 7 ring atoms, etc., up to and including 10 ring atoms. The term includes monocyclic or fused-ring polycyclic (i.e., rings which share adjacent pairs of ring atoms) groups. Unless stated otherwise specifically in the specification, an aryl moiety is optionally substituted by one or more substituents which are independently alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —ORa, —SRa, —OC(O)—Ra, —N(Ra)2, —C(O)Ra, —C(O)ORa, —OC(O)N(Ra)₂, —C(O)N(Ra)₂, —N(Ra)C(O)ORa, —N(Ra)C(O)Ra, —N(Ra)C(O)N(Ra)₂, —N(Ra)C(NRa)N(Ra)₂, —N(Ra)S(O)tRa (where t is 1 or 2), —S(O)tORa (where t is 1 or 2), —S(O)tN(Ra)₂ (where t is 1 or 2), or PO(Ra)₂, where each Ra is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl. Aromatic amino acids include those with a side chain comprising an aromatic group, such as such as phenylalanine, tyrosine, or tryptophan. Benzene, a 6-carbon ring containing three double bounds is a prototypical aromatic compound. Phenylalanine (Phe) and Tryptophan (Trp) are prototypical aromatic amino acids. Aryl may refer to an aromatic substituent and aryl-amine may refer to an aromatic group comprising an amine. An exemplary aromatic amine is aniline. Aromatic heterocycles refer to aromatic rings comprising cyclic ring structures comprising carbon and another atom, such as nitrogen, oxygen or sulfur. Nucleotide bases, such as adenine and cytosine, are exemplary aromatic heterocycles.

Biocompatible: Materials are considered biocompatible if they exert minimal destructive or host response effects while in contact with body fluids, cells, or tissues. A biocompatible group may contain chemical moieties, including from the following classes: aliphatic, alicyclic, heteroaliphatic, heteroalicyclic, aryl, or heteroaryl. However, depending on the molecular composition, such moieties are not always biocompatible.

The term “biocompatibility” is alternatively taken to mean either minimal interactions with recognition proteins and/or other components of biological systems (e.g., naturally occurring antibodies, cell proteins including glycoproteins, or cells); or substances and functional groups specifically intended to cause interactions with components of biological systems (e.g., drugs and prodrugs), such that the result of the interactions are not substantially negative or destructive.

Chemotherapeutic: Chemotherapeutic agents are chemical compounds useful in the treatment of cancer and include growth inhibitory agents or other cytotoxic agents and include alkylating agents, anti-metabolites, anti-microtubule inhibitors, topoisomerase inhibitors, receptor tyrosine kinase inhibitors, angiogenesis inhibitors and the like. Examples of chemotherapeutic agents include alkylating agents such as thiotepa and cyclosphosphamide (CYTOXAN®); alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphaoramide and trimethylolomelamine; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as aclacinomysins, actinomycin, authramycin, azaserine, bleomycins, cactinomycin, calicheamicin, carabicin, carminomycin, carzinophilin, chromomycins, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-FU; folic acid analogues such as denopterin, methotrexate, pteropterin, trimetrexate; purine analogues such as fludarabine, 6-mercaptopurine, thiamiprine, thioguanine; pyrimidine analogues such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; amsacrine; bestrabucil; bisantrene; edatraxate; defofamine; demecolcine; diaziquone; elfornithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; PSK®; razoxane; sizofiran; spirogermanium; tenuazonic acid; triaziquone; 2,2′,2″-trichlorotriethylamine; urethan; vindesine; dacarbazine; mannomustine; mitobronitol; mitolactol; pipobroman; gacytosine; arabinoside (“Ara-C”); cyclophosphamide; thiotepa; members of taxoid or taxane family, such as paclitaxel (TAXOL®docetaxel (TAXOTERE®) and analogues thereof; chlorambucil; gemcitabine; 6-thioguanine; mercaptopurine; methotrexate; platinum analogues such as cisplatin and carboplatin; vinblastine; platinum; etoposide (VP-16); ifosfamide; mitomycin C; mitoxantrone; vincristine; vinorelbine; navelbine; novantrone; teniposide; daunomycin; aminopterin; xeloda; ibandronate; CPT-11; topoisomerase inhibitor RFS 2000; difluoromethylornithine (DMFO); retinoic acid; esperamicins; capecitabine; inhibitors of receptor tyrosine kinases and/or angiogenesis, including sorafenib (NEXAVAR®), sunitinib (SUTENT®), pazopanib (VOTRIENT™), toceranib (PALLADIA™), vandetanib (ZACTIMA™), cediranib (RECENTIN®), regorafenib (BAY 73-4506), axitinib (AG013736), lestaurtinib (CEP-701), erlotinib (TARCEVA®), gefitinib (IRESSA™), BIBW 2992 (TOVOK™), lapatinib (TYKERB®), neratinib (HKI-272), and the like, and pharmaceutically acceptable salts, acids or derivatives of any of the above. Also included in this definition are anti-hormonal agents that act to regulate or inhibit hormone action on tumors such as anti-estrogens including for example tamoxifen, raloxifene, aromatase inhibiting 4(5)-imidazoles, 4-hydroxytamoxifen, trioxifene, keoxifene, LY 117018, onapristone, and toremifene (FARESTON®); and anti-androgens such as flutamide, nilutamide, bicalutamide, leuprolide, and goserelin; and pharmaceutically acceptable salts, acids or derivatives of any of the above. Other conventional cytotoxic chemical compounds as those disclosed in Wiemann et al., 1985, in Medical Oncology (Calabresi et al, eds.), Chapter 10, McMillan Publishing, are also suitable chemotherapeutic agents. Chemotherapeutics are sometimes referred to as chemotherapeutic drug molecules, cytotoxic drugs or more generically as drugs, drugs (D) or D.

Charge: A physical property of matter that affects its interactions with other atoms and molecules, including solutes and solvents. Charged matter experiences electrostatic force from other types of charged matter as well as molecules that do not hold a full integer value of charge, such as polar molecules. Two charged molecules of like charge repel each other, whereas two charged molecules of different charge attract each other. Charge is often described in positive or negative integer units.

The term “charged molecule”, abbreviated “C”, refers to any molecule that has one or more functional groups that are positively or negatively charged. The functional groups comprising the charged molecule may be partial or full integer values of charge. A charged molecule may be a molecule with a single charged functional group or multiple charged functional groups. Functional groups may be permanently charged or the functional groups comprising the charged molecule may have charge depending on the pH. The charged molecule may be comprised of positively charged functional groups, negatively charged functional groups or both positive and negatively charged functional groups. The net charge of the charged molecule may be positive, negative or neutral. The charge of a molecule can be readily estimated based on a molecule's Lewis structure and accepted methods known to those skilled in the art. Charge may result from inductive effects, e.g., atoms bonded together with differences in electron affinity may result in a polar covalent bond resulting in a partially negatively charged atom and a partially positively charged atom. For example, nitrogen bonded to hydrogen results in partial negative charge on nitrogen and a partial positive charge on the hydrogen atom. Alternatively, an atom may be considered to have a full integer value of charge when the number of electrons assigned to that atom is less than or equal to the atomic number of the atom. The charge of a functional group is determined by summing the charge of each atom comprising the functional group. The net charge of the charged molecule is determined by summing the charge of each atom comprising the molecule. Those skilled in the art are familiar with the process of estimating charge of a molecule, or individual functional groups, by summing the formal charge of each atom in a molecule or functional group, respectively.

Charged molecules may comprise negatively charged functional groups such as those that occur as the conjugate base of an acid at physiologic pH (e.g., functional groups with a pKa less than about 6.5), e.g., at a pH of about 7.4. These include but are not limited to molecules bearing carboxylates, sulfates, phosphates, phosphoramidates, and phosphonates. Charged molecules may comprise positively charged functional groups such as those that occur as the conjugate acid of a base at physiologic pH (e.g., functional groups wherein the pKa of the conjugate acid of a base is greater than about 8.5). These include but are not limited to molecules bearing primary, secondary and tertiary amines, as well as ammonium and guanidinium. Charged molecules may comprise functional groups with charge that is pH independent, including quaternary ammonium, phosphonium and sulfonium functional groups. In some embodiments, the charged molecule is a poly(amino acid) comprised of negatively or positively charged amino acids, or both negatively and positively charged amino acids. In some embodiments, the negatively charged amino acid is glutamic acid or aspartic acid. In other embodiments, the positively charged amino acid is lysine or arginine. In some embodiments the surface stabilizing group (S) comprises charged molecules (C). Those skilled in the art recognize that many such embodiments are possible. Specific compositions of charged molecules (C) suitable for compositions of amphiphilic block copolymers of the present disclosure are described throughout the specification.

Click chemistry reaction: A bio-orthogonal reaction that joins two compounds together under mild conditions in a high yield reaction that generates minimal, biocompatible and/or inoffensive byproducts. An exemplary click chemistry reaction used in the present disclosure is the reaction of an azide group with an alkyne to form a triazole linker through strain-promoted [3+2] azide-alkyne cyclo-addition.

Copolymer: A polymer derived from two (or more) monomeric species of polymer, as opposed to a homopolymer where only one monomer is used. Since a copolymer includes at least two types of constituent units (also structural units), copolymers may be classified based on how these units are arranged along the chain. The term “block copolymer” may be used herein to refer to a copolymer that comprises two or more homopolymer subunits linked by covalent bonds in which the union of the homopolymer subunits may require an intermediate non-repeating subunit, such as a junction block or linker. The term “block copolymer” may also be used herein to refer to a copolymer that comprises two or more copolymer subunits linked by covalent bonds in which the union of the copolymer subunits may require an intermediate non-repeating subunit, such as a junction block or linker. Block copolymers with two or three distinct blocks are referred to herein as “diblock copolymers” and “triblock copolymers,” respectively.

Delivery vehicle or carrier: Agent with no inherent therapeutic benefit but when combined with an agent for the purposes of drug delivery result in modification of the pharmaceutical compounds solution concentration, bioavailability, absorption, distribution and elimination for the benefit of improving product efficacy and safety, as well as patient convenience and compliance.

Drug: Any molecule—including, without limitation, proteins, peptides, sugars, saccharides, nucleosides, inorganic compounds, lipids, nucleic acids, small synthetic chemical compounds—that has a physiological effect when ingested or otherwise introduced into the body. A drug can be selected from a variety of known classes of drugs, including, for example, analgesics, anesthetics, anti-inflammatory agents, anthelmintics, anti-arrhythmic agents, antiasthma agents, antibiotics (including penicillins), anticancer agents (including Taxol), anticoagulants, antidepressants, antidiabetic agents, antiepileptics, antihistamines, antitussives, antihypertensive agents, antimuscarinic agents, antimycobacterial agents, antineoplastic agents, antioxidant agents, antipyretics, immunosuppressants, immunostimulants, antithyroid agents, antiviral agents, anxiolytic sedatives (hypnotics and neuroleptics), astringents, bacteriostatic agents, beta-adrenoceptor blocking agents, blood products and substitutes, bronchodilators, buffering agents, cardiac inotropic agents, chemotherapeutics, contrast media, corticosteroids, cough suppressants (expectorants and mucolytics), diagnostic agents, diagnostic imaging agents, diuretics, dopaminergics (antiparkinsonian agents), free radical scavenging agents, growth factors, haemostatics, immunological agents, lipid regulating agents, muscle relaxants, proteins, peptides and polypeptides, parasympathomimetics, parathyroid calcitonin and biphosphonates, prostaglandins, radio-pharmaceuticals, hormones, sex hormones (including steroids), time release binders, anti-allergic agents, stimulants and anoretics, steroids, sympathomimetics, thyroid agents, vaccines, vasodilators, and xanthines. Drug(s), may also be referred to as drug molecule(s), D, or drug(s) followed by the capital letter D, e.g., “drug(s) (D).”

Drug delivery: A method or process of administering a pharmaceutical compound to achieve a therapeutic effect in humans or animals.

Effective amount: The amount of a compound, material, or composition effective to achieve a particular biological result such as, but not limited to, biological results disclosed, described, or exemplified herein. Such results may include, but are not limited to, the effective reduction of symptoms associated with any of the disease states mentioned herein, as determined by any means suitable in the art.

Graft copolymer: A polymer that results from the linkage of a polymer of one composition to the side chains of a second polymer of a different composition. A first polymer linked through co-monomers to a second polymer is a graft copolymer. A first polymer linked through an end group to a second polymer may be described as a block polymer (e.g., A-B type di-block) or an end-grafted polymer.

Hydropathy index/GRAVY value: Is a number representing the hydrophobic or hydrophilic characteristics of an amino acid. There are a variety of scales that can be used to describe the relative hydrophobic and hydrophilic characteristics of amino acids comprising peptides. In the present disclosure, the Hydropathy scale of Kyte and Doolittle (Kyte J, Doolittle R F, J. Mol. Biol 157: 105-32, 1983) is used to calculate the grand average of hydropathy (GRAVY) value, sometimes referred to as the GRAVY score. The GRAVY value of a peptide is the sum of the Hydropathy values of all amino acids comprising the peptide divided by the length (i.e. number of amino acids) of the peptide. The GRAVY value is a relative value. The larger the GRAVY value, the more hydrophobic a peptide sequence is considered, whereas the lower the GRAVY value, the more hydrophilic a peptide sequence is considered.

Hydrophilic: Refers to the tendency of a material to disperse freely in aqueous media. A material is considered hydrophilic if it prefers interacting with other hydrophilic material and avoids interacting with hydrophobic material. In some cases, hydrophilicity may be used as a relative term, e.g., the same molecule could be described as hydrophilic or not depending on what it is being compared to. Hydrophilic molecules are often polar and/or charged and have good water solubility, e.g., are soluble up to 0.1 mg/mL or more.

Hydrophobic: Refers to the tendency of a material to avoid contact with water. A material is considered hydrophobic if it prefers interacting with other hydrophobic material and avoids interacting with hydrophilic material. Hydrophobicity is a relative term; the same molecule could be described as hydrophobic or not depending on what it is being compared to. Hydrophobic molecules are often non-polar and non-charged and have poor water solubility, e.g., are insoluble down to 0.1 mg/mL or less.

Hydrophobic polymer or oligomer (H): In the present disclosure, the terms “hydrophobic polymer or oligomer”, “hydrophobic polymer” or “hydrophobic oligomer” (H) is used as a general term to describe a molecule with limited water solubility, or amphiphilic characteristics, that can be linked to other groups to form a conjugate that forms particles in aqueous conditions. The hydrophobic polymer or oligomer (H) in this context promotes particle assembly due to its poor solubility, or tendency to assemble into particles, in aqueous conditions over certain temperatures and pH ranges.

Hydrophobic polymers or oligomers (H) as described herein are inclusive of amphiphilic molecules that may form supramolecular structures, such as micelles or bilayer-forming lamellar or multi-lamellar structures (e.g., polymersomes). The hydrophobic characteristics of the hydrophobic polymers or oligomers may be temperature- and/or pH-responsive. In some embodiments, the hydrophobic polymer or oligomer (H) is a polymer that is water soluble at low temperatures but is insoluble, or micelle-forming, at temperatures above, for example, 20° C., such as 20, 21, 22, 23, 24, 25, 26, 27, 28, 29, 30, 31, 32, 33, 34, 35, 36, 37, 38, 39 or 40° C. In other embodiments, the hydrophobic polymer or oligomer (H) is a polymer that is water soluble at low pH, for example, at a pH below 6.5 but insoluble, for example, at a pH above 6.5. Examples of hydrophobic polymers or oligomers (H) include but are not limited to polystyrene, poly(lactic-co-glycolic acid) (PLGA), as well as poly(amino acids) comprised of predominantly hydrophobic amino acids. In some embodiments, the hydrophobic polymer or oligomer (H) is a linear or branched poly(amino acid) comprised of 3 or more aromatic groups, such as 3 or more aromatic drug molecules. Specific compositions of hydrophobic polymers or oligomers (H) suitable for compositions of amphiphilic block copolymers of the present disclosure are described throughout the specification.

Immunomodulators: Substances that affect the functioning of the immune system. Immunomodulators may be stimulatory or inhibitory. Exemplary immunomodulators include macrolide drugs, such as rapamycin, which acts as an immunosuppressive agent through inhibition of mTOR. Corticosteroids and pattern recognition receptor agonists are additional classes of immunomodulators that have suppressive and stimulatory activity, respectively.

Immunostimulants: refers broadly to any substance that activate cells of the immune system. Immunostimulants include bacterial vaccines, colony stimulating factors, interferons, interleukins, and viral vaccines. Drug molecules with immunostimulatory properties include pattern recognition receptor (PRR) agonists. Non-limiting examples of pattern recognition receptor (PRR) agonists include TLR-1/2/6 agonists (e.g., lipopeptides and glycolipids, such as Pam2cys or Pam3cys lipopeptides); TLR-3 agonists (e.g., dsRNA, such as PolyI:C, and nucleotide base analogs); TLR-4 agonists (e.g., lipopolysaccharide (LPS) derivatives, for example, monophosphoryl lipid A (MPL) and small molecule is a derivative or analog of pyrimidoindole); TLR5 agonists (e.g., Flagellin); TLR-7 & -8 agonists (e.g., ssRNA and nucleotide base analogs, including derivatives of imidazoquinolines, hydroxy-adenine, benzonapthyridine and loxoribine); and TLR-9 agonists (e.g., unmethylated CpG); Stimulator of Interferon Genes (STING) agonists (e.g., cyclic dinucleotides, such as cyclic diadenylate monophosphate and amidobenzimidazole STING receptor agonists and its derivatives, such as those described in 2018, Ramanjulu J M, et al., Nature, 564:439-443); C-type lectin receptor (CLR) agonists (such as various mono, di, tri and polymeric sugars that can be linear or branched, e.g., mannose, Lewis-X tri-saccharides, etc.); RIG-I-like receptor (RLR) agonists; and NOD-like receptor (NLR) agonists (such as peptidogylcans and structural motifs from bacteria, e.g., meso-diaminopimelic acid and muramyl dipeptide); and combinations thereof. In several embodiments, the pattern recognition receptor agonist can be a TLR agonist, such as an imidazoquinoline-based TLR-7/8 agonist. For example, imidazoquinolines are synthetic immunostimulatory drugs that act by binding Toll-like receptors 7 and 8 (TLR-7/TLR-8) on antigen presenting cells (e.g., dendritic cells), structurally mimicking these receptors' natural ligand, viral single-stranded RNA. Imidazoquinolines are heterocyclic compounds comprising a fused quinoline-imidazole skeleton. Derivatives, salts (including hydrates, solvates, and N-oxides), and prodrugs thereof also are contemplated by the present disclosure. Particular imidazoquinoline compounds are known in the art, see for example, U.S. Pat. No. 6,518,265; and U.S. Pat. No. 4,689,338. Immunostimulants are sometimes referred to as immunostimulatory drug molecules, immunostimulant drug molecules, drugs with immunostimulatory properties or more generically as drugs, drugs (D) or D.

In vivo delivery: Administration of a composition, such as a composition comprising amphiphilic block copolymers and drug(s), by topical, transdermal, suppository (rectal, vaginal), pessary (vaginal), intravenous, oral, subcutaneous, intraperitoneal, intrathecal, intramuscular, intracranial, inhalational, oral, and the like, to a subject.

Linked or coupled: The terms “linked” and “coupled” mean joined together, either directly or indirectly. A first moiety may be covalently or noncovalently linked to a second moiety. In some embodiments, a first molecule is linked by a covalent bond to another molecule. In some embodiments, a first molecule is linked by electrostatic attraction to another molecule. In some embodiments, a first molecule is linked by dipole-dipole forces (for example, hydrogen bonding) to another molecule. In some embodiments, a first molecule is linked by van der Waals forces (also known as London forces) to another molecule. A first molecule may be linked by any and all combinations of such couplings to another molecule. The molecules may be linked indirectly, such as by using a linker. The molecules may be linked indirectly by interposition of a component that binds non-covalently to both molecules independently.

As used herein, “linked” and variations thereof, refer to maintaining molecules in chemical or physical association, including after immunization, at least until they contact a cell, particularly an immune cell. In some embodiments, linked components are associated so that the components are not freely dispersible from one another, at least until contacting a cell, such as an immune cell. For example, two components may be covalently linked to one another so that the two components are incapable of separately dispersing or diffusing.

Linker: A linker is a molecule or group of atoms that links or couples or joins together two or more moieties. In some embodiments, a linker precursor, referred to as “X1” may be present on one molecule and reacts with a linker precursor “X2” present on a heterologous molecule, resulting in the joining of the two molecules through a linker or linkage. In some embodiments, the hydrophobic polymer or oligomer (H) comprises a linker precursor X1 (X1-H) comprising a dibenzocyclooctyne (DBCO) and is reacted with a surface stabilizing group linked to a spacer and a linker precursor X2 (i.e. S-B-X2) comprising an azide, which results in triazole bond formation and the union of S-B-X2 and X1-H to form S-B-H.

Membrane: A spatially distinct collection of molecules that defines a 2-dimensional surface in 3-dimensional space, and thus separates one space from another in at least a local sense. A “bilayer membrane” or “bilayer(s)” is a self-assembled membrane of amphiphiles or super-amphiphiles in aqueous solutions.

Micelles: Spherical receptacles comprised of a single monolayer defining a closed compartment. Generally, amphiphilic molecules spontaneously form micellar structures in polar solvents. In contrast to liposome bilayers, micelles are “sided” in that they project a hydrophilic, polar outer surface and a hydrophobic interior.

Mol %: Refers to the percentage of a particular type of monomeric unit (or “monomer”) that is present in a polymer. For example, a polymer comprised of 100 monomeric units of A and B with a density (or “mol %”) of monomer A equal to 10 mol % would have 10 monomeric units of A, and the remaining 90 monomeric units (or “monomers”) may be monomer B or another monomer unless otherwise specified.

Monomeric unit: The term “monomeric unit” is used herein to mean a unit of polymer molecule containing the same or similar number of atoms as one of the monomers. Monomeric units, as used in this specification, may be of a single type (homogeneous) or a variety of types (heterogeneous). For example, poly(amino acids) are comprised of amino acid monomeric units. Monomeric units may also be referred to as monomers or monomer units or the like.

Net charge: The sum of electrostatic charges carried by a molecule or, if specified, a section of a molecule.

Particle: A nano- or micro-sized supramolecular structure comprised of an assembly of molecules. For example, in some embodiments, the amphiphilic block copolymer forms a particle in aqueous solution. In some embodiments, particle formation by the amphiphilic block copolymer is dependent on pH or temperature. In some embodiments, the nanoparticles comprised of amphiphilic block copolymers have an average diameter between 5 nanometers (nm) to 500 nm. In some embodiments, the nanoparticles comprised of amphiphilic block copolymer for micelles and have an average diameter between 5 nanometers (nm) to 50 nm, such as between 10 and 30 nm. In some embodiments, the nanoparticles comprised of amphiphilic block copolymers may be larger than 100 nm.

Peptide or polypeptide: Two or more natural or non-natural amino acid residues that are joined together through an amide bond. The amino acid residues may contain post-translational modification(s) (e.g., glycosylation and/or phosphorylation). Such modifications may mimic post-translational modifications that occur naturally in vivo or may be non-natural. Any one or more of the components of the amphiphilic block copolymers may be comprised of peptides.

There is no conceptual upper limit on the length of a peptide. The length of the peptide is typically selected depending on the application. In several embodiments, the hydrophobic polymer or oligomer (H) is comprised of a peptide that can be between 3 to 1,000 amino acids in total, typically no more than 300 amino acids in total (e.g., 300 amino acids in length for linear peptides).

Peptide Modifications: Peptides may be altered or otherwise synthesized with one or more of several modifications as set forth below. In addition, analogs (non-peptide organic molecules), derivatives (chemically functionalized peptide molecules obtained starting from a peptide) and variants (homologs) of these peptides can be utilized in the methods described herein. The peptides described herein are comprised of a sequence of amino acids, analogs, derivatives, and variants, which may be either L- and/or D-versions. Such peptides may contain peptides, analogs, derivatives, and variants that are naturally occurring and otherwise.

Peptides can be modified through a variety of chemical techniques to produce derivatives having essentially the same activity as the unmodified peptides, and optionally having other desirable properties. For example, carboxylic acid groups of the peptide, whether at the carboxyl terminus or at a side chain, can be provided in the form of a salt of a pharmaceutically-acceptable cation or esterified to form a CC₁-CC₁₆ ester, wherein CC refers to a carbon chain (and thus, CC1 refers to a single carbon and CC16 refers to 16 carbons), or converted to an amide. Amino groups of the peptide, whether at the amino terminus or at a side chain, can be in the form of a pharmaceutically-acceptable acid addition salt, such as the HCl, HBr, acetic, trifluoroacetic, formic, benzoic, toluene sulfonic, maleic, tartaric and other organic salts, or can be modified or converted to an amide.

Peptides may be modified to contain substituent groups that contain a positive or negative charge or both. The positive and/or negative charge may be affected by the pH at which the peptide is present.

Hydroxyl groups of the peptide side chains may be converted to CC₁-CC₁₆ alkoxy or to a CC₁-CC₁₆ ester using well-recognized techniques, or the hydroxyl groups may be converted (e.g., sulfated or phosphorylated) to introduce negative charge. Phenyl and phenolic rings of the peptide side chains may be substituted with one or more halogen atoms, such as fluorine, chlorine, bromine or iodine, or with CC₁-CC₁₆ alkyl, CC₁-CC₁₆ alkoxy, carboxylic acids and esters thereof, or amides of such carboxylic acids. Methylene groups of the peptide side chains can be extended to homologous CC₂-CC₄ alkylenes. Thiols can be used to form disulfide bonds or thioethers, for example through reaction with a maleimide. Thiols may be protected with any one of a number of well-recognized protecting groups, such as acetamide groups. Those skilled in the art will also recognize methods for introducing cyclic structures into the peptides of this invention to select and provide conformational constraints to the structure that result in enhanced stability. Reference may be made to Greene et al., “Greene's Protective Groups in Organic Synthesis” Fourth Edition, John Wiley & Sons, Inc. 2006 for details of additional modifications that can be made to functional groups.

Pharmaceutically acceptable vehicles: The pharmaceutically acceptable carriers (vehicles) useful in this disclosure are conventional. Remington's Pharmaceutical Sciences, by E. W. Martin, Mack Publishing Co., Easton, Pa., 15th Edition (1975), describes compositions and formulations suitable for pharmaceutical delivery of one or more therapeutic compositions, such as one or more therapeutic cancer vaccines, and additional pharmaceutical agents.

In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. For solid compositions (for example, powder, pill, tablet, or capsule forms), conventional non-toxic solid carriers can include, for example, pharmaceutical grades of mannitol, lactose, starch, or magnesium stearate. In addition to biologically-neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate.

Plurality: The word “plurality” is used herein to mean more than one.

Polar: A description of the properties of matter. Polar is a relative term, and may describe a molecule or a portion of a molecule that has partial charge that arises from differences in electronegativity between atoms bonded together in a molecule, such as the bond between nitrogen and hydrogen. Polar molecules prefer interacting with other polar molecules and typically do not associate with non-polar molecules. In specific, non-limiting cases, a polar group may contain a hydroxyl group, or an amino group, or a carboxyl group, or a charged group. In specific, non-limiting cases, a polar group may prefer interacting with a polar solvent such as water. In specific, non-limiting cases, introduction of additional polar groups may increase the solubility of a portion of a molecule.

Polymer: A molecule containing repeating structural units (monomers). As described in greater detail throughout the disclosure, polymers may be used for any number of components of the amphiphilic block copolymer and may be natural or synthetic. In certain embodiments, a hydrophobic or amphiphilic polymer is used as the hydrophobic polymer (H) and drives particle assembly. The polymers included in the disclosed embodiments can form polymer nanoparticles that can be administrated to a subject without causing adverse side effects. The polymers included in the disclosed embodiments can form polymer nanoparticles that can be administered to a subject to cause an immune response or to treat and/or ameliorate a disease. The polymers included in the disclosed embodiments may include a side chain with a functional group that can be utilized, for example, to facilitate linkage to a drug molecule. In several embodiments, the polymer can contain two or more polymer blocks linked through a linker to create a block copolymer, such as an amphiphilic diblock copolymer. In several embodiments, a polymer block may be predominantly hydrophobic in character. In several embodiments, the polymer consists of peptides, their analogs, derivatives, and variants. Various compositions of polymers useful for the practice of the invention are discussed in greater detail elsewhere.

Polymerization: A chemical reaction, usually carried out with a catalyst, heat or light, in which monomers combine to form a chainlike, branched or cross-linked macromolecule (a polymer). The chains, branches or cross-linked macromolecules can be further modified by additional chemical synthesis using the appropriate substituent groups and chemical reactions. The monomers may contain reactive substances. Polymerization commonly occurs by addition or condensation. Addition polymerization occurs when an initiator, usually a free radical, reacts with a double bond in the monomer. The free radical adds to one side of the double bond, producing a free electron on the other side. This free electron then reacts with another monomer, and the chain becomes self-propagating, thus adding one monomer unit at a time to the end of a growing chain. Condensation polymerization involves the reaction of two monomers resulting in the splitting out of a water molecule. In other forms of polymerization, a monomer is added one at a time to a growing chain through the staged introduction of activated monomers, such as during solid phase peptide synthesis.

Polymersome: Vesicle, which is assembled from synthetic multi-block polymers in aqueous solutions. Unlike liposomes, a polymersome does not include lipids or phospholipids as its majority component. Consequently, polymersomes can be thermally, mechanically, and chemically distinct and, in particular, more durable and resilient than the most stable of lipid vesicles. The polymersomes assemble during processes of lamellar swelling, e.g., by film or bulk rehydration or through an additional phoresis step, as described below, or by other known methods. Like liposomes, polymersomes form by “self-assembly,” a spontaneous, entropy-driven process of preparing a closed semi-permeable membrane.

Purified: A substance or composition that is relatively free of impurities or substances that adulterate or contaminate the substance or composition. The term purified is a relative term and does not require absolute purity. Substantial purification denotes purification from impurities. A substantially purified substance or composition is at least 60%, 70%, 80%, 90%, 95%, 98%, or 99% pure.

Soluble: Capable of becoming molecularly or ionically dispersed in a solvent to form a homogeneous solution. When referring to a peptide, a soluble peptide is understood to be a single molecule in solution that does not assemble into multimers or other supramolecular structures through hydrophobic or other non-covalent interactions. A soluble molecule is understood to be freely dispersed as single molecules in solution. Hydrophobic polymers or oligomers (H) described herein are insoluble down to about 0.1 mg/mL or less. Solubility can be determined by visual inspection, by turbidity measurements or by dynamic light scattering.

Spacer: The term spacer (denoted B) is used herein to describe molecules that function to join together and provide distance, i.e. space, between the hydrophobic polymer or oligomer (H) and the surface stabilizing group (S). In some embodiments, the spacer modulates the rate of degradation of the amphiphilic block copolymers. In other embodiments, the spacer functions to impart hydrophobic or hydrophilic properties to modulate micelle or polymersome stability. In still other embodiments, the composition of the spacer used as a linker may be selected to impart rigidity or flexibility. Specific compositions of spacers (S) suitable for compositions of amphiphilic block copolymers of the present disclosure are described throughout the specification.

Subject and patient: These terms may be used interchangeably herein to refer to both human and non-human animals, including birds and non-human mammals, such as rodents (for example, mice and rats), non-human primates (for example, rhesus macaques), companion animals (for example domesticated dogs and cats), livestock (for example pigs, sheep, cows, llamas, and camels), as well as non-domesticated animals (for example big cats).

Treating, preventing, or ameliorating a disease: “Treating” refers to an intervention that reduces a sign or symptom or marker of a disease or pathological condition after it has begun to develop. For example, treating a disease may result in a reduction in tumor burden, meaning a decrease in the number or size of tumors and/or metastases, or treating a disease may result in immune tolerance that reduces systems associated with autoimmunity. “Preventing” a disease refers to inhibiting the full development of a disease. A disease may be prevented from developing at all. A disease may be prevented from developing in severity or extent or kind. “Ameliorating” refers to the reduction in the number or severity of signs or symptoms or marker of a disease, such as cancer.

Reducing a sign or symptom or marker of a disease or pathological condition related to a disease, refers to any observable beneficial effect of the treatment and/or any observable effect on a proximal, surrogate endpoint, for example, tumor volume, whether symptomatic or not. Reducing a sign or symptom associated with a tumor or viral infection can be evidenced, for example, by a delayed onset of clinical symptoms of the disease in a susceptible subject (such as a subject having a tumor which has not yet metastasized, or a subject that may be exposed to a viral infection), a reduction in severity of some or all clinical symptoms of the disease, a slower progression of the disease (for example by prolonging the life of a subject having a tumor or viral infection), a reduction in the number of relapses of the disease, an improvement in the overall health or well-being of the subject, or by other parameters well known in the art (e.g., that are specific to a particular tumor or viral infection). A “prophylactic” treatment is a treatment administered to a subject who does not exhibit signs of a disease or exhibits only early signs for the purpose of decreasing the risk or severity of developing pathology.

Tumor or cancer or neoplastic: An abnormal growth of cells, which can be benign or malignant, often but not always causing clinical symptoms. “Neoplastic” cell growth refers to cell growth that is not responsive to physiologic cues, such as growth and inhibitory factors.

A “tumor” is a collection of neoplastic cells. In most cases, tumor refers to a collection of neoplastic cells that forms a solid mass. Such tumors may be referred to as solid tumors. In some cases, neoplastic cells may not form a solid mass, such as the case with some leukemias. In such cases, the collection of neoplastic cells may be referred to as a liquid cancer.

Cancer refers to a malignant growth of neoplastic cells, being either solid or liquid. Features of a cancer that define it as malignant include metastasis, interference with the normal functioning of neighboring cells, release of cytokines or other secretory products at abnormal levels and suppression or aggravation of inflammatory or immunological response(s), invasion of surrounding or distant tissues or organs, such as lymph nodes, etc.

A tumor that does not present substantial adverse clinical symptoms and/or is slow growing is referred to as “benign.”

“Malignant” means causing, or likely to cause in the future, significant clinical symptoms. A tumor that invades the surrounding tissue and/or metastasizes and/or produces substantial clinical symptoms through production and secretion of chemical mediators having an effect on nearby or distant body systems is referred to as “malignant.”

“Metastatic disease” refers to cancer cells that have left the original tumor site and migrated to other parts of the body, for example via the bloodstream, via the lymphatic system, or via body cavities, such as the peritoneal cavity or thoracic cavity.

The amount of a tumor in an individual is the “tumor burden”. The tumor burden can be measured as the number, volume, or mass of the tumor, and is often assessed by physical examination, radiological imaging, or pathological examination.

An “established” or “existing” tumor is a tumor that exists at the time a therapy is initiated. Often, an established tumor can be discerned by diagnostic tests. In some embodiments, an established tumor can be palpated. In some embodiments, an established tumor is at least 500 mm³, such as at least 600 mm³, at least 700 mm³, or at least 800 mm³ in size. In other embodiments, the tumor is at least 1 cm long. With regard to a solid tumor, an established tumor generally has a newly established and robust blood supply, and may have induced the regulatory T cells (Tregs) and myeloid derived suppressor cells (MDSC).

Unit dose: A discrete amount of the pharmaceutical composition comprising a predetermined amount of the active ingredient.

Vesicle: A fluid filled sac. In some embodiments the vesicle is a sac comprising an amphiphilic substance. In some embodiments, the sac is a nanoparticle-based vesicle, which refers to a vesicle with a size or dimensions in the nanometer range. In some embodiments, a polymer vesicle is a vesicle that is manufactured with one or more polymers.

A person of ordinary skill in the art would recognize that the definitions provided above are not intended to include impermissible substitution patterns (e.g., methyl substituted with 5 different groups, and the like). Such impermissible substitution patterns are easily recognized by a person of ordinary skill in the art. Any functional group disclosed herein and/or defined above can be substituted or unsubstituted, unless otherwise indicated herein. Unless otherwise explained, all technical and scientific terms used herein have the same meaning as commonly understood by one of ordinary skill in the art to which this disclosure belongs. The term “comprises” means “includes.” Therefore, comprising “A” or “B” refers to including A, including B, or including both A and B. It is further to be understood that all base sizes or amino acid sizes, and all molecular weight or molecular mass values, given for nucleic acids or polypeptides are approximate, and are provided for description. Although methods and materials similar or equivalent to those described herein can be used in the practice or testing of the present disclosure, suitable methods and materials are described herein. In case of conflict, the present specification, including explanations of terms, will control. In addition, the materials, methods, and examples are illustrative only and not intended to be limiting.

Provided herein is an amphiphilic block copolymer having any one of the formulas, S-[B]-H, S-[B]-H(D), D-[B]-H, S-B(D)-H, S-[B]-H-[B]-S, S-[B]-H(D)-[B]-S, D-[B]-H-[B]-S, D-[B]-H-[B]-D, S-B(D)-H-[B]-S or S-B(D)-H-B(D)-S; wherein S is a hydrophilic surface stabilizing group; B is a spacer group; H is a hydrophobic polymer or oligomer; D is a drug molecule; ( ) denotes that the group is bonded directly or indirectly as a side chain or as part of a side chain group to the adjacent group; [ ] denotes that the group is optional; and - denotes that each of the adjacent S, B, H or D are linked directly to one another or indirectly to one another via a linker group

The amphiphilic block copolymers described herein have many uses but have particular utility in the formation of micelles, polymersomes or encapsulating membranes to encapsulate one or more drug(s) (D) and any other agents as required, such as dye molecules or radiotracers. The “loaded” micelle or polymersome may be further used to transport an encapsulatable material (an “encapsulant”) to or from its immediately surrounding environment. For example, the micelle or polymersome can be used to deliver a drug or therapeutic composition to a patient's tissue or through the blood stream.

Chemotherapeutic drug molecules (D) admixed with the amphiphilic block copolymer (e.g., S-B-H+D) lead to improved tumor clearance and/or reduced toxicity as compared with D alone.

Chemotherapeutic drug molecules (D) conjugated to the hydrophobic polymer or oligomer (H) of the amphiphilic block copolymer (e.g., S-B-H(D)) lead to improved tumor clearance and/or reduced toxicity as compared with D alone.

Chemotherapeutic drug molecules (D) conjugated to the hydrophobic polymer or oligomer (H) and admixed with a micelle-forming amphiphilic block copolymer (e.g., S-[B]-H+D-H) form stable micelles and lead to improved tumor clearance and/or reduced toxicity as compared with D alone.

Immunostimulant drug molecules (D) admixed with the amphiphilic block copolymer (e.g., S—B-H+D) lead to improved tumor clearance and/or reduced toxicity as compared with D alone.

Immunostimulant drug molecules (D) conjugated to the hydrophobic polymer or oligomer (H) of the amphiphilic block copolymer (e.g., S-[B]-H(D)) lead to improved tumor clearance and/or reduced toxicity as compared with D alone.

Immunostimulant drug molecules (D) conjugated to the hydrophobic polymer or oligomer (H) and admixed with a micelle-forming amphiphilic block copolymer (e.g., S-[B]-H+D-H) form stable micelles and lead to improved tumor clearance and/or reduced toxicity as compared with D alone.

Particles may be formed comprising a single composition of amphiphilic block copolymer, wherein the drug molecule, D, is non-covalently incorporated within the particle or directly linked to the particle through covalent attachment to the amphiphilic block copolymer:
S-[B]-H+D
S-[B]-H(D)
S(D)-[B]-H
S-B(D)-H
S-[B]-H-[B]-S+D
S-[B]-H(D)-[B]-S
S-B(D)-H-[B]-S
S-B(D)-H-B(D)-S

Incorporation of drug molecules to particles based on amphiphilic block copolymers can be improved by, e.g., attachment of the drug molecule to a hydrophobic polymer or oligomer, H, to yield D-H, which can be used in the preparation of mosaic particles comprising S-[B]-H+D-H.

In some examples, drug molecules linked to a hydrophobic polymer or oligomer may be too hydrophobic for practical use and may require the attachment of a stabilizing group S, to yield S-[B]-H(D) or S-B(D)-H, which can be used in the preparation of mosaic particles. Non-limiting examples include: S-[B]-H+S-[B]-H(D) and S-[B]-H+S-B(D)-H.

Alternatively, the drug molecule may be hydrophilic and substitute the surface stabilizing group, such as D-[B]-H.

The amphiphilic block copolymers described herein can also be used to prepare “empty” micelles or polymersomes.

The amphiphilic block copolymers described herein can also be used to control the release of an encapsulated material from a micelle or polymersome by modulating and controlling the micelle or polymersome stability and surface properties.

In embodiments in which the drug molecule is linked to the amphiphilic block copolymer through a covalent bond, the rate of release of the drug molecule from the micelle or polymersome may be modulated by varying the composition of linker molecule.

The amphiphilic block copolymer can be a diblock, triblock, or other multi-block copolymer, which may each be referred generically as block copolymers. Each block serves to segregate the hydrophilic and hydrophobic characteristics to provide polarity to the amphiphile. The architecture of each block may be the same or different. In some embodiments, the amphiphilic block copolymer comprises of two or more linear blocks that are attached end-to-end. In other embodiments, a branched copolymer block is attached to a linear copolymer block. In other embodiments, a branched copolymer block is attached to a branched copolymer block. In some embodiments, the amphiphilic block copolymer is a brush copolymer, such as a brush copolymer formed by grafting multiple polymer arms to a linear copolymer. In other embodiments, the amphiphilic block copolymer comprises a linear or branched copolymer block linked to a brush copolymer block. In preferred embodiments, the amphiphilic block copolymer comprises a brush S-[B] linked to a linear or branched hydrophobic polymer or oligomer (H). A non-limiting example is a linear or branched hydrophobic polymer or oligomer (H) attached to an amplifying linker that is attached to 2 or more, such as between 2 and 8, hydrophilic linear polymers as spacers (B) that are each linked to a surface stabilizing group (S).

In certain embodiments, the hydrophobic polymer or oligomer (H) comprises 3 or more cyclic aromatic groups. In certain embodiments, the aromatic groups comprising the hydrophobic polymer or oligomer (H) are heterocyclic aromatic groups. In still other embodiments, the aromatic or heterocyclic aromatic groups comprising the hydrophobic polymer or oligomer (H) further comprise an aromatic amine (or “aryl amine”). The present inventors have surprisingly found that hydrophobic polymers or oligomers comprising aromatic amines lead to improved manufacturability and solubility in water-miscible solvents, compared with polymers comprised of aromatic groups without an amine. The present inventors have also found that amphiphilic block copolymers comprised of hydrophobic polymers or oligomers comprising aromatic amines lead to formation of stable particles with low critical micellar concentration (CMC).

The hydrophobic polymer or oligomer (H) is a molecule with substantially limited water solubility, or is amphiphilic in properties, and capable of assembling into supramolecular structures, e.g., micellar, nano- or micro-particles in aqueous conditions. In certain embodiments, the hydrophobic polymer or oligomer (H) is insoluble, or forms micelles, in aqueous conditions down to about 0.1 mg/mL or about 0.01 mg/mL.

The hydrophobic polymer or oligomer (H) may comprise a linear, branched or brush polymer. The hydrophobic polymer or oligomer (H) can be a homopolymer or copolymer. The hydrophobic polymer or oligomer (H) can be comprised of one or many different types of monomer units. The hydrophobic polymer or oligomer (H) can be a statistical copolymer or alternating copolymer. The hydrophobic polymer or oligomer (H) can be a block copolymer, such as the A-B type, or the polymer can be comprised of a grafted copolymer, whereby two or more polymers are linked through polymer analogous reaction.

The hydrophobic polymer or oligomer (H) may comprise polymers comprising naturally occurring and/or non-natural monomers and combinations thereof.

Natural biopolymers may include peptides (sometimes referred to as poly(amino acids)) comprised of amino acids; a specific example is poly(tryptophan). Natural biopolymers that are water soluble in their native form may be used but must be modified chemically to make such natural biopolymers water insoluble and suitable for use as hydrophobic polymers or oligomers (H). For example, biopolymers comprised of hydrophilic amino acids, such as glutamic acid or lysine residues may be modified at the gamma carboxyl or epsilon amine groups, respectively, for the attachment of a hydrophobic molecule, such as a hydrophobic drug molecule, i.e. D, to increase the hydrophobicity of the resulting modified biopolymer. Similarly, biopolymers can be selected from hydrophilic polysaccharides, which may include but are not limited to glycogen, cellulose, dextran, alginate and chitosan, but such polysaccharides should be modified chemically, for example via acetylation or benzoylation of hydrophilic functional groups to render the resulting modified polysaccharide water insoluble.

In additional embodiments, the hydrophobic polymer or oligomer (H) is a polymer that may include monomers of (meth)acrylates, (meth)acrylamides, styryl and vinyl moieties. Specific examples of (meth)acrylates, (meth)acrylamides, as well as styryl- and vinyl-based monomers include N−2-hydroxypropyl(methacrylamide) (HPMA), hydroxyethyl(methacrylate) (HEMA), Styrene and vinylpyrrolidone (PVP), respectively. For example, hydrophobic polymers or oligomers (H) comprised of HPMA, which is hydrophilic, may additionally comprise hydrophobic co-monomers, such as N-benzyl methacrylamide, which increase the hydrophobic characteristics of the copolymer. In still other embodiments, the hydrophobic polymer or oligomer (H) may be comprised of monomers of lactic acid, glycolic acid, ethylene oxide, propylene oxide or combinations thereof.

The hydrophobic polymer or oligomer (H) can be a thermoresponsive polymer comprised of monomers of N-isopropylacrylamide (NIPAAm); N-isopropylmethacrylamide (NIPMAm); N,N′-diethylacrylamide (DEAAm); N-(L)-(1-hydroxymethyl)propyl methacrylamide (HMPMAm); N,N′-dimethylethylmethacrylate (DMEMA), 2-(2-methoxyethoxy)ethyl methacrylate (DEGMA), triethylene glycol methyl ether methacrylate (TEGMA) or y-(2-methoxyethoxy)esteryl-L-glutamate.

In preferred embodiments, the hydrophobic polymer or oligomer is comprised of monomers that comprise aromatic groups. In preferred embodiments, the hydrophobic polymer or oligomer (H) is a poly(amino acid) comprised of amino acids that comprise aromatic groups. Exemplary aromatic groups (sometimes referred to as “aromatics” or “aromatic rings”) include but are not limited to benzene and fused benzene ring structures or heterocyclic aromatic molecules. Non-limiting examples include:

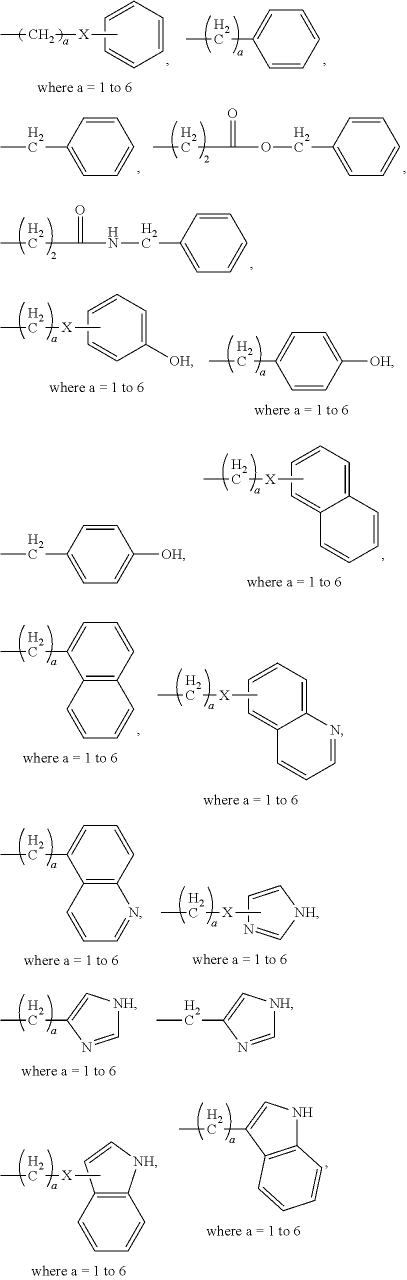

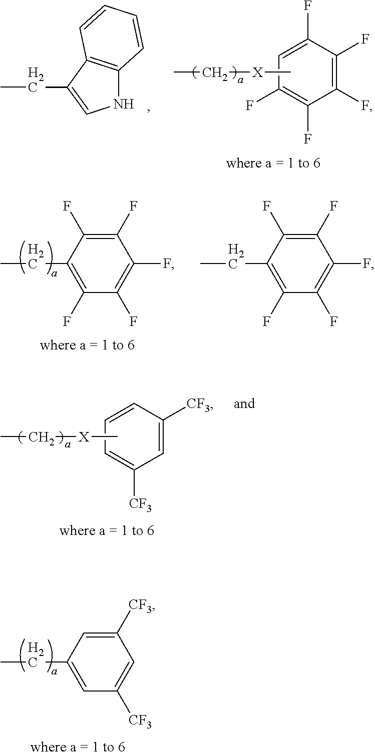

wherein X is any suitable linker molecule and a is an integer value, typically between 1 and 6. Furthermore, in the aforementioned aromatic groups one or more hydrogen atoms may be substituted for one or more fluorine atoms.

The present inventors have unexpectedly found that poly(amino acid)-based hydrophobic polymers or oligomers (H) comprising aromatic groups further comprising an aryl amine lead to improved manufacturing and solubility in water-miscible solvents compared with poly(amino acids) comprised of aromatic groups without an amine.

In certain embodiments, the hydrophobic polymer or oligomer (H) is a poly(amino acid) comprising aromatic amines of the formula —Ar—NHR, where Ar can be a C6-C10 or heterocyclic aromatic group, optionally fused to another ring, and R is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl. Non-limiting examples of aromatic groups comprising an aryl amine include but are not limited to:

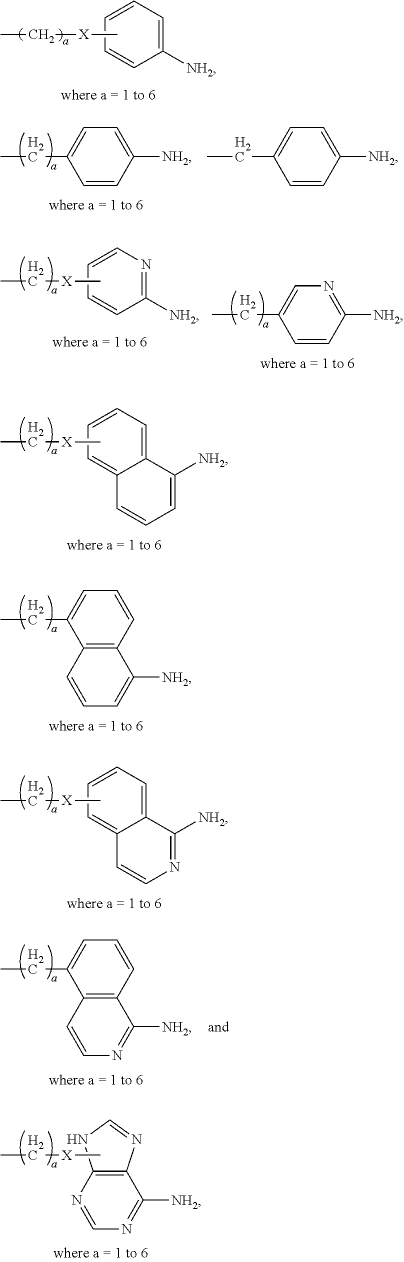

wherein X is any suitable linker molecule and a is an integer value, typically between 1 and 6.

The hydrophobic polymer or oligomer (H) may be a poly(amino acid)-based polymer comprised of four different classes of co-monomers, namely hydrophobic monomers ( [symbol] ), spacer monomers (m), charged amino acid monomers (n) for charge compensation, and functional group containing monomers (o) for drug molecule (D) attachment. The different co-monomers may be included in the hydrophobic polymer or oligomer (H) for different reasons. Hydrophobic monomers ( [symbol] ) comprising aromatic containing amino acids are selected to increase the hydrophobic properties of the backbone. Spacer monomers (m), such as ethylene oxide based amino acids, glycine, serine and alanine can be selected to increase spacing between monomer units. Charged co-monomers (n) are selected to balance a charged drug molecule such that the overall charge of the hydrophobic polymer or oligomer (H) is zero. Functional group containing monomers (o) are used for drug molecule attachment.

The hydrophobic polymer or oligomer (H) may be a poly(amino acid)-based polymer comprising one or more hydrophobic monomers ( [symbol] ), optionally one or more spacer monomers (m), optionally one or more charged amino acid monomers (n), and optionally one or more drug molecule (D) containing monomers (o). The hydrophobic monomers, spacer monomers, charged amino acid monomers and drug molecule (D) containing monomers can be assembled in any combination and any order.

In some embodiments, the hydrophobic polymer or oligomer (H) is a poly(amino acid)-based polymer that has the formula:

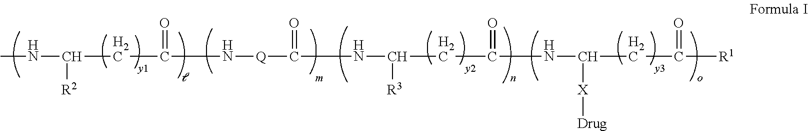

The poly(amino acid)-based polymer of Formula I typically comprises the monomer [symbol] and optional monomers, m, n and o. R¹ is typically selected from one of hydrogen, hydroxyl or amine. In some embodiments, R¹ is linked to a drug molecule. The number of methylene units denoted by y1, y2, y3, is typically 0 to 6, such as 0, 1, 2, 3, 4, 5, or 6. The N-terminal amine of the poly(amino acid) of Formula I is typically linked to a surface stabilizing group (S) either directly or via a spacer (B) through any suitable linker molecule. In some embodiments, the N-terminal amine is linked to a drug molecule (D) either directly or via a spacer (B) through any suitable linker molecule. In typical embodiments, the poly(amino acid)-based polymer of Formula I comprises monomer(s) [symbol] that are selected from any natural or non-natural amino acid wherein R² is selected from aromatic groups that endow the polymer backbone with hydrophobic properties. In some embodiments, the R² included in Formula I can be selected from

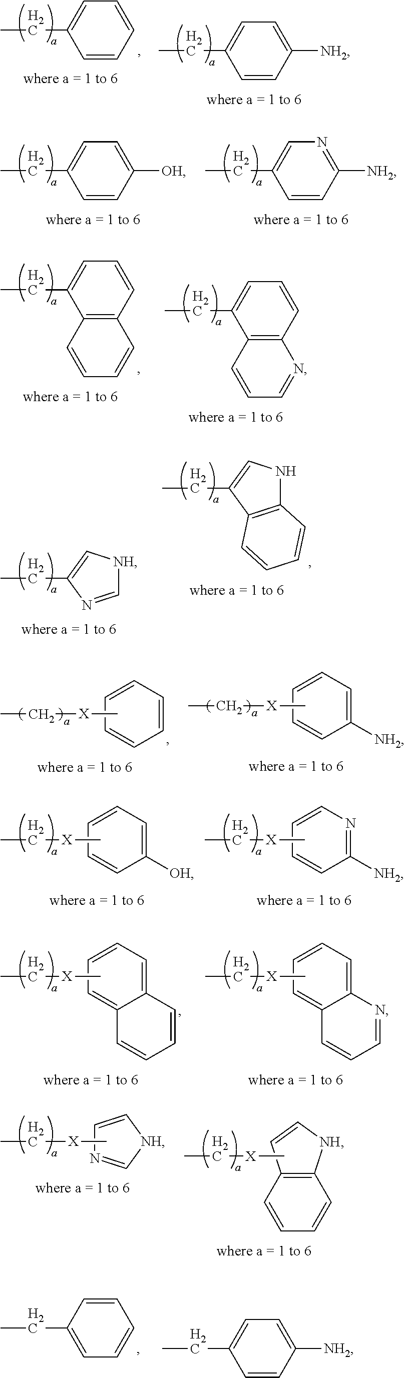

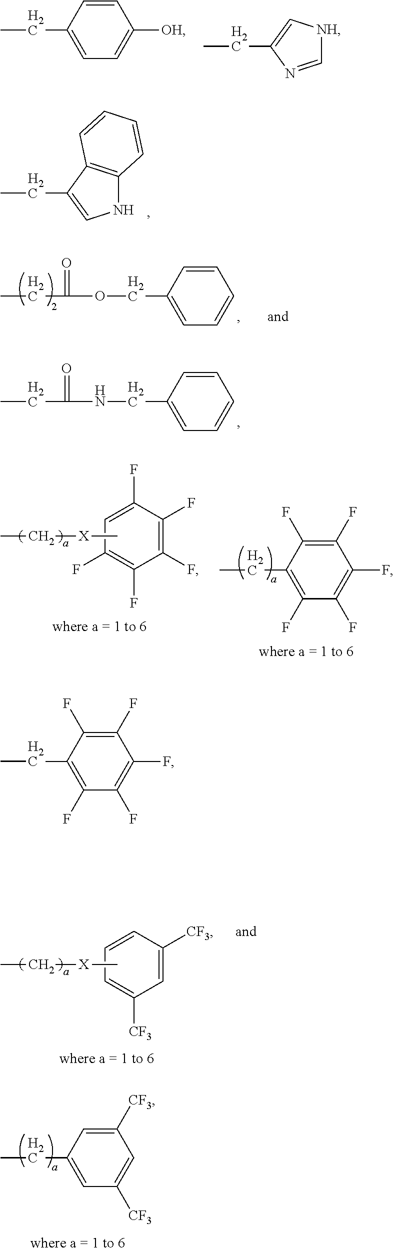

wherein X is any suitable linker.

In some embodiments, the poly(amino acid)-based polymer of Formula I comprises optional co-monomer(s) m that are selected from any natural or non-natural amino acid, such as a PEG amino acid spacer (e.g., m of Formula I is —NH—(CH₂—CH₂—O)_{y4}-(CH₂)_{y5}-(CO)—, wherein y4 is an integer typically between 1 and 24 and y5 is an integer typically between 1 and 3) or an amino acid with a small substituent selected from Hydrogen, lower alkyl or a lower alkyl comprising a hydroxyl and is provided to increase the spacing or flexibility of the polymer backbone.

In some embodiments, the poly(amino acid)-based polymer of Formula I comprises optional co-monomer(s), n, that are selected from any natural or non-natural amino acid, wherein R³ is selected from any group comprising a functional group that carriers charge either permanently or at a specific pH. In some embodiments, the R³ included in Formula II can be selected from

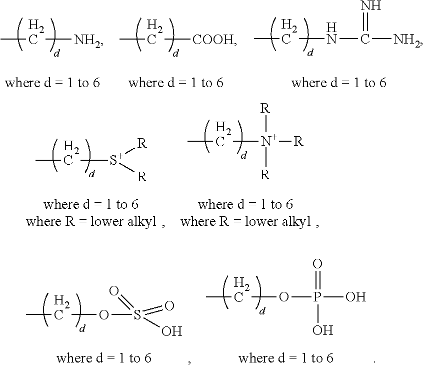

In some embodiments, the poly(amino acid)-based polymer of Formula I comprises optional co-monomer(s) o that are selected from any natural or non-natural amino acid, wherein a drug molecule (D) is linked through any suitable linker, X, to the monomer o. Non-limiting examples of drug molecules (D) linked to co-monomer o include immunostimulants and anti-neoplastic compounds. The drug molecule (D) linked to poly(amino acids) of Formula I may be hydrophobic, hydrophilic, amphiphilic, charged or neutral in properties.

In some embodiments of poly(amino acid)-based hydrophobic polymers or oligomers (H) of Formula (I), the integers y1, y2, and y3 are equal to 0 and Formula I reduces to:

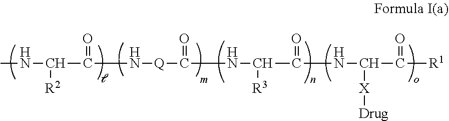

In certain embodiments, the linker, X, is comprised of an alkyl chain with a Functional Group (FG) that is used to link a drug molecule (D) to the polymer backbone. Formula I(b) can thus be elaborated to give Formula I(b).

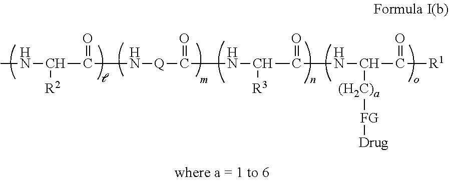

In Formula I(b), the integer, a, is typically 0 to 6 such as 0, 1, 2, 3, 4, 5, or 6. The Functional Group (FG) included in Formula I(b) is typically selected from carboxylic acid, amine, thiol, aldehyde, ketone, hydrazine, azide, or alkyne. In certain embodiments, the FG links the drug molecule (D or “Drug”) to the poly(amino acid) backbone either directly or through a linker.

For clarity, any references to Formula I disclosed herein refer to any possible embodiment of poly(amino acids) of Formula I, including Formula I, Formula I(a) and Formula I(b).

In some embodiments, the hydrophobic polymer or oligomer (H) is a poly(amino acid) of Formula I comprised entirely of monomers of [symbol] :

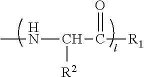

Non-limiting examples include:

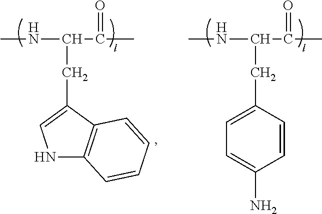

A non-limiting example of a hydrophobic polymer or oligomer (H) comprised of a poly(amino acid) of Formula I(b) comprised entirely of [symbol] co-monomers selected from tryptophan, wherein [symbol] is equal to 5 (i.e. 5 monomeric units), R1 is an amine and the N-terminal amine is linked to a surface stabilizing group (S) either directly or indirectly through a spacer (B) and/or linker, is shown here for clarity:

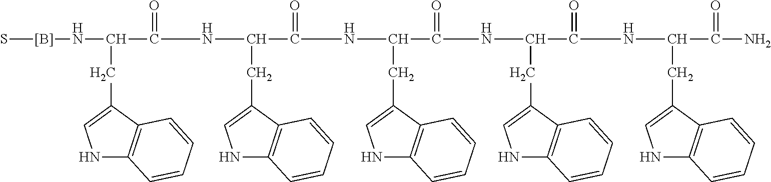

A non-limiting example of a hydrophobic polymer or oligomer (H) comprised of a poly(amino acid) of Formula I(b) comprised entirely of [symbol] co-monomers selected from para-amino-phenylalanine (sometimes referred to as amino phenylalanine; CAS no. 943-80-6), wherein [symbol] is equal to 10 (i.e. 10 monomeric units), R1 is an amine and the N-terminal amine is linked to a surface stabilizing group (S) either directly or indirectly through a spacer (B) and/or linker, is shown here for clarity:

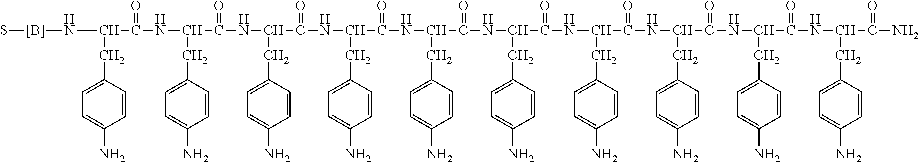

Herein, we report the unexpected finding that amphiphilic copolymers with hydrophobic polymers or oligomers (H) comprised of poly(amino acid)-based copolymers that include aromatic amino acids (e.g., phenylalanine, amino phenylalanine, histidine, tryptophan, tyrosine, benzyl glutamate) and/or aromatic drug molecules (e.g., imidazoquinolines), have unexpected improvements in manufacturability through improved organic solvent solubility and improved particle stability as compared with poly(amino acids) predominantly comprised of aliphatic amino acids. Thus, in certain embodiments, hydrophobic polymers or oligomers (H) comprised of poly(amino acids), or other classes of polymers, include one or more, typically 3 or more, aromatic groups optionally comprising an aromatic amine.

An additional notable finding relates to how the number of monomer units comprising the hydrophobic polymer or oligomer (H) impacts particle formation by amphiphilic block copolymers. For example, poly(amino acid)-based hydrophobic polymers or oligomers (H) comprised of at least 5 hydrophobic amino acids were typically needed to promote particle formation of amphiphilic block copolymers comprising S and H. Though, unexpectedly, poly(amino acid)-based hydrophobic polymers or oligomers (H) comprised of oligomers with as few as 3 monomers that included aromatic rings were found to be sufficient to drive stable particle, e.g., micelle, assembly when linked to a surface stabilizing group, S, either directly or through a spacer, B. Notably, increasing the number of monomers comprising the hydrophobic polymer or oligomer (H) from 3 to 5 and from 5 to 10 hydrophobic monomers increased the strength of the forces promoting particle formation, leading to more stable and larger particles formed by the amphiphilic block copolymers described herein. The present inventors have surprisingly found that hydrophobic polymers or oligomers (H) comprising poly(amino acid)-based hydrophobic polymers or oligomers (H) of between about 3 to about 30 aromatic amino acids (i.e. amino acids that comprise aromatic groups) form stable nanoparticle micelles as amphiphilic block copolymers with diverse S and B compositions (i.e. S-B-H). Therefore, in certain embodiments, the hydrophobic polymer or oligomer (H) is selected from poly(amino acids) that comprise from about 3 to about 30 aromatic amino acids.

While there is no theoretical upper limit for the number of monomers that can be included in the hydrophobic polymer or oligomer (H), it was unexpectedly found that linear poly(amino acids) with 40 or more amino acids that comprise aromatic groups were more challenging to manufacture and—when incorporated in amphiphilic block copolymers—resulted in less stable particles than those with fewer, i.e. those with 3 to 30 amino acids. It was also found unexpectedly that the preferred number of monomers is also dependent on the architecture of the poly(amino acid)-based hydrophobic polymer or oligomer (H). Accordingly, preferred embodiments of hydrophobic polymers or oligomers (H) comprised of linear poly(amino acids), such as those produced by solid phase peptide synthesis, comprise between 3 to 30 amino acids. Preferred embodiments of hydrophobic polymers or oligomers (H) comprised of branched poly(amino acids) produced by solid phase peptide synthesis, solution phase peptide synthesis or a combination thereof comprise between 3 to 31 amino acids. Preferred embodiments of hydrophobic polymers or oligomers (H) comprised of brush poly(amino acids), which may be produced by grafting one or more peptides to the side chains and/or ends of a linear or branched peptide either on-resin during solid phase peptide synthesis or in solution phase, comprise between 3 to 100 amino acids.

In certain embodiments, the poly(amino acid)-based hydrophobic polymer or oligomer (H) may comprise two or more different monomers. In such embodiments, the mol % of each of the co-monomers should be selected to meet the specific demands of the application. For instance, the poyl(amino acid)-based hydrophobic polymer or oligomer (H) should include a sufficient mol % of hydrophobic amino acids that comprise aromatic groups to ensure stable particle formation when such hydrophobic polymer or oligomer (H) is included in an amphiphilic block copolymer, e.g., as S-[B]-H. Importantly, it was unexpectedly found that the density (or mol %) of hydrophobic amino acids that comprise aromatic groups required to ensure stable particle formation depends on the total number of amino acids comprising the poly(amino acid). In general, the density (mol %) of hydrophobic amino acids required is inversely proportional to the size of the poly(amino acid)-based hydrophobic polymer or oligomer (H); i.e. a higher density (mol %) is required for smaller poly(amino acids) comprised of fewer amino acids than larger poly(amino acids) that comprise a greater number of amino acids. For instance, the density (mol %) of hydrophobic amino acids that comprise aromatic groups should be 100 mol % for poly(amino acids) with 3 amino acids; 80-100 mol % for poly(amino acids) with 4 amino acids, such as 80 mol %, 81 mol %, 82 mol %, 83 mol %, 84 mol %, 85 mol %, 86 mol %, 87 mol %, 88 mol %, 89 mol %, 90 mol %, 91 mol %, 92 mol %, 93 mol %, 94 mol %, 95 mol %, 96 mol %, 97 mol %, 98 mol %, 99 mol % or 100 mol % for poly(amino acids) with 4 amino acids; 60-100 mol % for poly(amino acids) with between 5 and 10 amino acids, such as 60 mol %, 61 mol %, 62 mol %, 63 mol %, 64 mol %, 65 mol %, 66 mol %, 67 mol %, 68 mol %, 69 mol %, 70 mol %, 71 mol %, 72 mol %, 73 mol %, 74 mol %, 75 mol %, 76 mol %, 77 mol %, 78 mol %, 79 mol %, 80 mol %, 81 mol %, 82 mol %, 83 mol %, 84 mol %, 85 mol %, 86 mol %, 87 mol %, 88 mol %, 89 mol %, 90 mol %, 91 mol %, 92 mol %, 93 mol %, 94 mol %, 95 mol %, 96 mol %, 97 mol %, 98 mol %, 99 mol % or 100 mol % for poly(amino acids) with between 5 and 10 amino acids; and, 40-100 mol % for poly(amino acids) with between 11 and 100 amino acids, such as 40 mol %, 41 mol %, 42 mol %, 43 mol %, 44 mol %, 45 mol %, 46 mol %, 47 mol %, 48 mol %, 49 mol %, 60 mol %, 51 mol %, 52 mol %, 53 mol %, 54 mol %, 55 mol %, 56 mol %, 57 mol %, 58 mol %, 59 mol %, 60 mol %, 61 mol %, 62 mol %, 63 mol %, 64 mol %, 65 mol %, 66 mol %, 67 mol %, 68 mol %, 69 mol %, 70 mol %, 71 mol %, 72 mol %, 73 mol %, 74 mol %, 75 mol %, 76 mol %, 77 mol %, 78 mol %, 79 mol %, 80 mol %, 81 mol %, 82 mol %, 83 mol %, 84 mol %, 85 mol %, 86 mol %, 87 mol %, 88 mol %, 89 mol %, 90 mol %, 91 mol %, 92 mol %, 93 mol %, 94 mol %, 95 mol %, 96 mol %, 97 mol %, 98 mol %, 99 mol % or 100 mol % for poly(amino acids) with between 11 and 100 amino acids.

In some embodiments, the amphiphilic block copolymer comprises a poly(amino acid)-based hydrophobic polymer or oligomer (H) that is branched. In preferred embodiments, the branched poly(amino acid) comprises a monomer with three or more functional groups. In some embodiments, one of the functional groups is selected from an amine, and the other two or more functional groups are selected from carboxylic acid. In other embodiments, one the functional groups is selected from a carboxylic acid, and the other two or more functional groups are selected from amine. A branched poly(amino acid) comprised of monomers with 3 functional groups has two branches per monomer, which can each incorporate another monomer, which may be the same or different. For a branched poly(amino acid) comprised entirely of amino acids with 3 functional groups, each monomer splits into two branches that can incorporate another two monomers that can each incorporate another two monomers and so on. In this way, the branched poly(amino acid) expands from a single amino acid (or focal point) and terminates with a number of functional groups equal to the generation number multiplied by the number of branches per generation. For example, in some embodiments, the poly(amino acid)-based hydrophobic polymer or oligomer (H) has two branch points for each generation, wherein each branch is terminated with a functional group, sometimes referred to as an end group or attachment point. For example, generation 2 would comprise 3 monomers with 4 attachment points; generation 3 would comprise 7 monomers with 8 attachment points; and, generation 4 would comprise 15 monomers with 16 attachment points. It was found unexpectedly that amphiphilic block copolymers comprised of branched poly(amino acid)-based hydrophobic polymers or oligomers (H) linked at the focal point to a surface stabilizing group (S) either directly or indirectly through a spacer (B) and/or linker, formed more stable particles when each of the terminal attachment points was linked to an aromatic group, such as an aromatic drug molecule (D). Therefore, in preferred embodiments, each of the terminal attachment points for branched poly(amino acid)-based hydrophobic polymers or oligomers (H) are linked to an aromatic group and the focal point of the branched poly(amino acid) is linked to the surface stabilizing group (S) either directly or indirectly through a spacer (B) and/or linker through a functional group accessible on the focal point monomer. In some embodiments of amphiphilic block copolymers, the hydrophobic polymer or oligomer (H) comprises a branched poly(amino acid) comprised of 3 monomers linked to 4 aromatic groups, such as aromatic drug molecules (D). In some embodiments of amphiphilic block copolymers, the hydrophobic polymer or oligomer (H) comprises a branched poly(amino acid) comprised of 7 monomers linked to 8 aromatic groups, such as aromatic drug molecules (D). In still other embodiments of amphiphilic block copolymers, the hydrophobic polymer or oligomer (H) comprises a branched poly(amino acid) comprised of 15 monomers linked to 16 aromatic groups, such as aromatic drug molecules (D).

Based on the aforementioned unexpected findings, the percentage of monomers, [symbol] , m, n and o comprising the poly(amino acid)-based polymer of Formula I can be rationally selected to meet the demands of specific applications. For instance, in some embodiments, the amphiphilic block copolymer comprises a hydrophobic polymer or oligomer comprised of poly(amino acid)-based polymers of Formula I that are comprised entirely of the monomer [symbol] , and therefore, drug molecules (D) must be admixed with the amphiphilic block copolymers, i.e. S-[B]-H+D, for particle encapsulation.

In some embodiments, the amphiphilic block copolymer comprises a hydrophobic polymer or oligomer comprised of a poly(amino acid)-based polymer of Formula I comprised of the monomer o, which is linked to drug molecules (D), and optionally co-monomers, [symbol] , m and n. In some embodiments, the drug molecule linked to monomers o is hydrophobic and the poly(amino acid) is comprised entirely of the monomer o (i.e. 100 mol %), or a combination of [symbol] and o, typically between 1-99 mol % [symbol] and 1-99 mol %, such as 10-20 mol % [symbol] and 80-90 mol % o, 30-40 mol % [symbol] and 60-70 mol % o; 40-50 mol % [symbol] and 50-60 mol % o; 60-70 mol % [symbol] and 30-40 mol % o; 70-80 mol % [symbol] and 20-30 mol % o; and 80-90 mol % [symbol] and 10-20 mol % o. An unexpected finding was that higher densities mol % of co-monomer o led to enhanced biological activity. Therefore, preferred embodiments of poly(amino acid)-based polymers of Formula I are comprised entirely of the monomer o. In other embodiments, wherein the poly(amino acid)-based polymers of Formula I is comprised of the monomers o and [symbol] , the density (mol %) of o is typically greater than 10 mol %, such as 10, 20, 30, 40 50, 60, 70, 80 or 90 mol %.

In other embodiments, the drug molecule linked to monomer o is hydrophilic and therefore co-monomers [symbol] are needed to ensure particle formation of the amphiphilic block copolymer. The density (mol %) of [symbol] required to induce particle formation of poly(amino acid)-based polymers of Formula I comprised of [symbol] and o, wherein o is linked to a hydrophilic drug molecule, depends on the total number of amino acids; the density (mol %) of [symbol] should be 80 mol % for poly(amino acids) with 4 amino acids; 60 mol % or greater for poly(amino acids) with between 5 and 10 amino acids; and, 40 mol % or greater for poly(amino acids) with between 11 and 100 amino acids.

In some embodiments, the poly(amino acid)-based polymer of Formula I comprises co-monomers [symbol] and m, wherein m provides space, i.e. distance, between the hydrophobic monomers [symbol] and may reduce polymer rigidity. In other embodiments, the poly(amino acid)-based polymer of Formula I comprises monomers [symbol] , m and o wherein monomers m provide space between the bulky substituents comprising monomers [symbol] and o. In still other embodiments, the poly(amino acid)-based polymer of Formula I is comprised entirely of monomers m and o.

In other embodiments, the poly(amino acid)-based polymer of Formula I comprises monomers [symbol] and o and optionally monomers m and n, wherein monomer n is used to modulate the charge of the polymer backbone. For instance, drug molecules may comprise charge at physiologic pH (pH˜7.4), which can disrupt particle formation by promoting solubility of the hydrophobic polymer or oligomer (H). Co-monomers n are introduced to neutralize charge present on drug molecules linked to co-monomer o, thereby providing an overall neutral charge. In some embodiments the poly(amino acid)-based polymer of Formula I is comprised entirely of monomers m, n and o, or just n and o. In still other embodiments, the poly(amino acid)-based polymer of Formula I comprises monomers [symbol] , m, n and o.

The average molecular weight of the polymer comprising the poly(amino acid)-based hydrophobic polymer or oligomer (H) can be readily estimated based on the number and composition of amino acids and is typically between about 500 g/mol to about 20,000 g/mol. In some embodiments, the polymer molecular weight is between about 1,000 and 5,000, or between about 5,000 and 10,000, or between about 10,000 and 20,000 g/mol.

The polydispersity, Mw/Mn, of the hydrophobic polymer or oligomer (H) typically ranges from about 1.0 to 2.0 and depends on the polymerization technique used. For instance, poly(amino acid)-based hydrophobic polymers or oligomers (H) are typically prepared by solid phase peptide synthesis and will have polydispersity of 1.0 as the polymers are molecularly defined. Polymers formed by chain growth polymerization will have polydispersities >1.0. The hydrophobic polymer or oligomer (H) may also comprise polymers based on cyclic monomers, such as poly(amino acid)-based hydrophobic polymers or oligomers (H) based on amino acid N-carboxyanhydrides (NCAs).

In certain embodiments, poly(amino acid)-based hydrophobic polymers or oligomers (H) are synthesized by solid-phase peptide synthesis. Peptide (or “poly(amino acid)”)-based polymers comprising amino acids with aromatic rings, such as tryptophan, though hydrophobic in aqueous conditions, provided unexpected improvements in manufacturing by solid-phase synthesis as compared with hydrophobic peptides (or “poly(amino acids)”) without aromatic rings. Surprisingly, poly(amino acids) further comprising aromatic amines provided still further improvements in manufacturability as compared with poly(amino acids) comprising aromatic rings or heterocyclic aromatic rings. Thus, in certain embodiments, hydrophobic polymers or oligomers (H) based on peptides produced by solid phase synthesis include amino acids comprising aromatic rings further comprising aryl amine groups.

The present inventors have surprisingly found that hydrophobic polymers or oligomers (H) comprising aromatic amines have improved solubility in water-miscible organic solvents as compared with aliphatic hydrophobic polymers. This improved solubility in water-miscible organic solvents leads to improved manufacturability.

The present inventors have also found that increasing the total number of monomers of hydrophobic polymers or oligomers (H) (e.g., length for linear polymers) leads to improved kinetic stability and that only certain compositions of hydrophobic peptides greater than 10 amino acids can be reliably produced by solid phase peptide synthesis (SPPS). For example, peptides comprising between 10-30 consecutive monomers of poly(para-amino phenylalanine) can be produced by SPPS, but peptides with 10-30 consecutive monomers based on phenylalanine, tryptophan or aliphatic amino acids, e.g., leucine, or valine, cannot be accessed readily by SPPS and/or are not readily soluble in water-miscible organic solvents.

In some embodiments, the hydrophobic polymer or oligomer (H) is a poly(amino acid) that is linked to drug molecules (D), such as PRR agonists through side chains of the poly(amino acid), such as through co-monomer o for poly(amino acid)-based polymers of Formula I. In other embodiments, the hydrophobic polymer or oligomer (H) comprises a poly(amino acid) wherein a drug molecule (D) with anti-neoplastic properties (i.e. chemotherapeutic) is attached to side groups distributed along the backbone of the poly(amino acid), such as through co-monomer o for poly(amino acid)-based polymers of Formula I. The drug molecule with anti-neoplastic properties may either be hydrophobic or hydrophilic, charged or uncharged in properties. In certain embodiments, the drug molecule with anti-neoplastic properties is selected from anthracyclines, taxanes and platinum-based compounds.

In some embodiments, the drug molecule (D) linked to the hydrophobic polymer or oligomer (H) is hydrophobic and promotes increased micelle or polymersome stability. In other embodiments, the drug molecule (D) that is linked to the hydrophobic polymer or oligomer (H) comprises an aromatic or heterocyclic aromatic ring. In some embodiments, the drug molecule (D) that is linked to the polymer-based hydrophobic polymer or oligomer (H) comprises an aromatic ring further comprising an aryl amine (i.e. Ar—NH₂). In some embodiments, the drug molecule (D) attached to the hydrophobic polymer or oligomer (H) comprises a heterocyclic aromatic ring further comprising an aryl amine. In embodiments wherein the hydrophobic polymer or oligomer (H) comprises a drug molecule (D) that comprises an aromatic group, optionally comprising a heterocycle and/or aryl amine, we report the unexpected finding that such hydrophobic polymer or oligomer (H) are highly soluble in pharmaceutically acceptable organic solvents, such as DMSO and ethanol, but insoluble in aqueous buffers.

In some embodiments, the drug molecule (D) that is linked to the poly(amino acid)-based hydrophobic polymer or oligomer (H), such as through co-monomer o for poly(amino acid)-based polymers of Formula I, comprises an aromatic ring structure. The monomers linked to drug molecules (D) that comprise aromatic groups (or “an aromatic ring structure”) may be referred to as aromatic amino acids or amino acids (or monomers) that comprise an aromatic group. For example, a non-limiting example of a poly(amino acid) comprised of 5 monomers, 3 of which are linked to drug molecules (D) that comprise aromatic groups, can be described as having 60 mol % monomers with aromatic groups. A non-limiting example of a poly(amino acid) comprised of 5 monomers, 5 of which are linked to drug molecules (D) that comprise aromatic groups, can be described as having 100 mol % monomers with aromatic groups. A non-limiting example of a poly(amino acid) comprised of 10 monomers, 5 of which are linked to drug molecules (D) that comprise aromatic groups, can be described as having 50 mol % monomers with aromatic groups.

The density of the drug molecule (D) linked to the hydrophobic polymer or oligomer (H) can be varied as needed for particular applications. Drug molecules (D) may be linked to the hydrophobic polymer or oligomer at densities ranging from 1 to 100 mol %, such as from 1 to 10 mol % or from 50-100 mol %. Mol % refers to the percentage of monomers comprising the polymer that are linked to the drug molecule (D). For example, 10 mol % drug molecule (D) is equal to 10 monomer units linked to the drug molecule from a total of 100 monomer units, where the remaining 90 may be macromolecule-forming monomeric units, which are not linked to the drug molecule (D). For hydrophobic drug molecules (D) with aromatic ring structures, the preferred density (mol %) is typically greater than 20 mol % for linear polymers, such as between 20-100 mol %, and full occupancy of terminal attachment points for branched polymers.

In some embodiments, the hydrophobic polymer or oligomer (H) comprises a poly(amino acid) wherein a drug molecule (D) with immunostimulatory properties (i.e. immunostimulant) is attached to side groups distributed along the backbone of the poly(amino acid). The drug molecule with immunostimulatory properties may either be hydrophobic or hydrophilic, charged or uncharged in properties. In certain embodiments, the drug molecule with immunostimulatory properties is a PRR agonist.

In several embodiments, the drug molecule with immunostimulatory properties can be a pattern recognition receptor (PRR) agonist. Non-limiting examples of pattern recognition receptor (PRR) agonists include TLR-1/2/6 agonists (e.g., lipopeptides and glycolipids, such as Pam2cys or Pam3cys lipopeptides); TLR-3 agonists (e.g., dsRNA, such as PolyI:C, and nucleotide base analogs); TLR-4 agonists (e.g., lipopolysaccharide (LPS) derivatives, for example, monophosphoryl lipid A (MPL) and small molecule derivatives or analogs of pyrimidoindole); TLR5 agonists (e.g., Flagellin); TLR-7 & -8 agonists (e.g., ssRNA and nucleotide base analogs, including derivatives of imidazoquinolines, hydroxy-adenine, benzonapthyridine and loxoribine); and TLR-9 agonists (e.g., unmethylated CpG); Stimulator of Interferon Genes (STING) agonists (e.g., cyclic dinucleotides, such as cyclic diadenylate monophosphate); C-type lectin receptor (CLR) agonists (such as various mono, di, tri and polymeric sugars that can be linear or branched, e.g., mannose, Lewis-X tri-saccharides, etc.); RIG-I-like receptor (RLR) agonists; and NOD-like receptor (NLR) agonists (such as peptidogylcans and structural motifs from bacteria, e.g., meso-diaminopimelic acid and muramyl dipeptide); and combinations thereof. In several embodiments, the pattern recognition receptor agonist can be a TLR agonist, such as an imidazoquinoline-based TLR-7/8 agonist. For example, the Ligand with adjuvant properties can be Imiquimod (R837) or Resiquimod (R848), which are approved by the FDA for human use.

In several embodiments, the drug molecule with immunostimulatory properties can be a TLR-7 agonist, a TLR-8 agonist and/or a TLR-7/8 agonist. Numerous such agonists are known, including many different imidazoquinoline compounds.

Imidazoquinolines are of use in the methods disclosed herein. Imidazoquinolines are synthetic immunomodulatory drugs that act by binding Toll-like receptors 7 and 8 (TLR-7/TLR-8) on antigen presenting cells (e.g., dendritic cells), structurally mimicking these receptors' natural ligand, viral single-stranded RNA. Imidazoquinolines are heterocyclic compounds comprising a fused quinoline-imidazole skeleton. Derivatives, salts (including hydrates, solvates, and N-oxides), and prodrugs thereof also are contemplated by the present disclosure. Particular imidazoquinoline compounds are known in the art, see for example, U.S. Pat. Nos. 6,518,265; and 4,689,338. In some non-limiting embodiments, the imidazoquinoline compound is not imiquimod and/or is not resiquimod.

In some embodiments, the drug molecule with immunostimulatory properties can be a small molecule having a 2-aminopyridine fused to a five membered nitrogen-containing heterocyclic ring, including but not limited to imidazoquinoline amines and substituted imidazoquinoline amines such as, for example, amide substituted imidazoquinoline amines, sulfonamide substituted imidazoquinoline amines, urea substituted imidazoquinoline amines, aryl ether substituted imidazoquinoline amines, heterocyclic ether substituted imidazoquinoline amines, amido ether substituted imidazoquinoline amines, sulfonamido ether substituted imidazoquinoline amines, urea substituted imidazoquinoline ethers, thioether substituted imidazoquinoline amines, hydroxylamine substituted imidazoquinoline amines, oxime substituted imidazoquinoline amines, 6-, 7-, 8-, or 9-aryl, heteroaryl, aryloxy or arylalkyleneoxy substituted imidazoquinoline amines, and imidazoquinoline diamines; tetrahydroimidazoquinoline amines including but not limited to amide substituted tetrahydroimidazoquinoline amines, sulfonamide substituted tetrahydroimidazoquinoline amines, urea substituted tetrahydroimidazoquinoline amines, aryl ether substituted tetrahydroimidazoquinoline amines, heterocyclic ether substituted tetrahydroimidazoquinoline amines, amido ether substituted tetrahydroimidazoquinoline amines, sulfonamido ether substituted tetrahydroimidazoquinoline amines, urea substituted tetrahydroimidazoquinoline ethers, thioether substituted tetrahydroimidazoquinoline amines, hydroxylamine substituted tetrahydroimidazoquinoline amines, oxime substituted tetrahydroimidazoquinoline amines, and tetrahydroimidazoquinoline diamines; imidazopyridine amines including but not limited to amide substituted imidazopyridine amines, sulfonamide substituted imidazopyridine amines, urea substituted imidazopyridine amines, aryl ether substituted imidazopyridine amines, heterocyclic ether substituted imidazopyridine amines, amido ether substituted imidazopyridine amines, sulfonamido ether substituted imidazopyridine amines, urea substituted imidazopyridine ethers, and thioether substituted imidazopyridine amines; 1,2-bridged imidazoquinoline amines; 6,7-fused cycloalkylimidazopyridine amines; imidazonaphthyridine amines; tetrahydroimidazonaphthyridine amines; oxazoloquinoline amines; thiazoloquinoline amines; oxazolopyridine amines; thiazolopyridine amines; oxazolonaphthyridine amines; thiazolonaphthyridine amines; pyrazolopyridine amines; pyrazoloquinoline amines; tetrahydropyrazoloquinoline amines; pyrazolonaphthyridine amines; tetrahydropyrazolonaphthyridine amines; and 1H-imidazo dimers fused to pyridine amines, quinoline amines, tetrahydroquinoline amines, naphthyridine amines, or tetrahydronaphthyridine amines.

In some embodiments, the drug molecule with immunostimulatory properties is an imidazoquinoline with the formula:

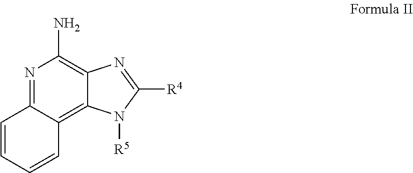

In Formula II, R⁴ is selected from one of hydrogen, optionally-substituted lower alkyl, or optionally-substituted lower ether; and R⁵ is selected from one of optionally substituted arylamine, or optionally substituted lower alkylamine. R⁴ may be optionally substituted to a linker that links to a polymer.

In some embodiments, the R⁴ included in Formula II can be selected from hydrogen,

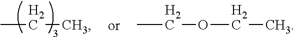

In some embodiments, R⁵ can be selected from,

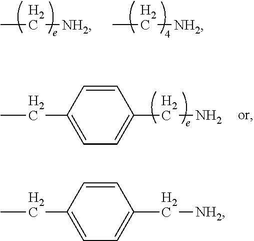

wherein e denotes the number of methylene units and is an integer from 1 to 4.

In some embodiments, R⁵ can be

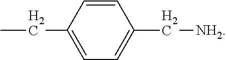

In some embodiments, R⁵ can be

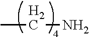

In some embodiments, R⁴ can be

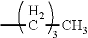

### and R⁵ can be

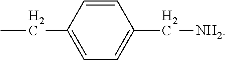

Non-limiting examples of hydrophobic polymers or oligomers (H) comprised of poly(amino acids) of Formula I linked to drug molecules of Formula II include:

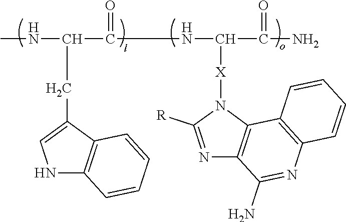

wherein [symbol] is typically between 3-300 and o is between 3-300. For example, when [symbol] =2 and o=3, the PGP-55. RT. peptide is comprised of amino acids, wherein 3 of the amino acids are linked to drug molecules of Formula II.

A non-limiting example of a hydrophobic polymer or oligomer (H) comprised of a poly(amino acid) of Formula I linked to drug molecules of Formula II, wherein [symbol] is equal to 2 (i.e. 2 monomeric units of tryptophan), o is equal to 3 (i.e. 3 monomer units linked to drug molecules), RT is an amine and the N-terminal amine is linked to a surface stabilizing group (S) either directly or indirectly through a spacer (B) and/or linker, is shown here for clarity:

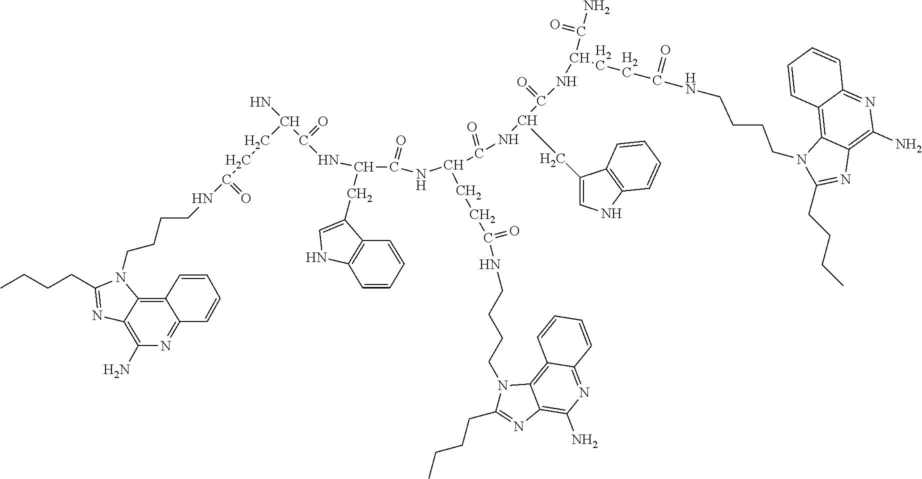

In some embodiments the hydrophobic polymer or oligomer (H) is a branched poly(amino acid), wherein each of the end groups (or attachment points) is occupied by aromatic groups, such as drug molecules (D) that comprise an aromatic group.

A non-limiting example of a hydrophobic polymer or oligomer (H) comprised of a branched poly(amino acid) linked to drug molecules of Formula II, wherein the focal point (first generation monomer) is linked to a surface stabilizing group (S) either directly or indirectly through a spacer (B) and/or linker is shown here for clarity:

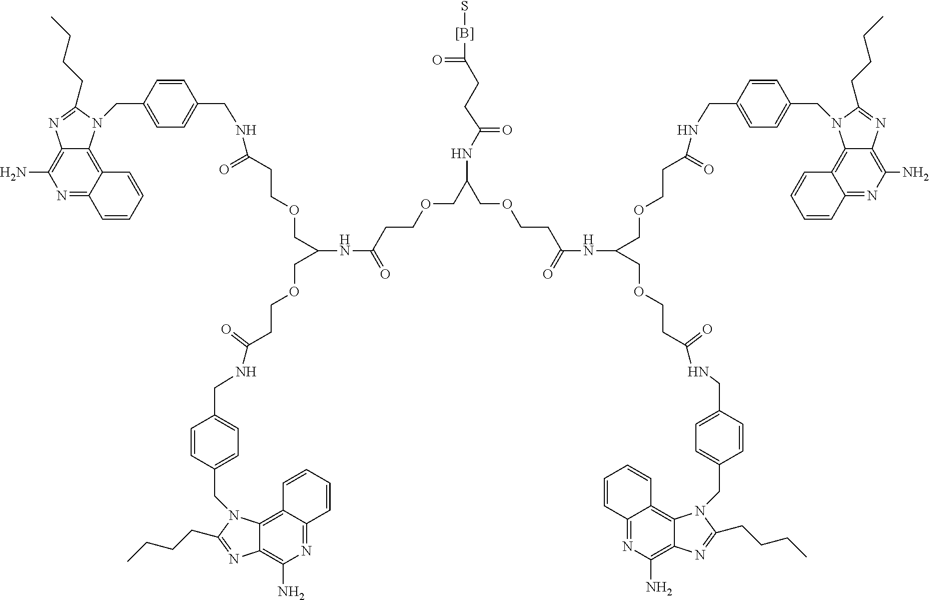

In certain embodiments, the hydrophilic surface stabilizing group (S) comprises one or more charged functional groups. In certain of these embodiments, the surface stabilizing group provides a high net charge (>+4, or <−4). The present inventors have surprisingly found that S groups comprising charged functional groups with a high net charge (>+4, or <−4) ensure stable nanoparticle formation.

In certain embodiments, the surface stabilizing group may comprise a charged molecule (C) (sometimes referred to as a “charged moiety”), which refers to any molecule that has one or more functional groups that are positively or negatively charged in aqueous buffers at a pH of about 7.4. The functional groups comprising the charged molecule (C) may be partial or full integer values of charge. A charged molecule (C) may be a molecule with a single charged functional group or multiple charged functional groups. The net charge of the charged molecule (C) may be positive, negative or neutral. The charge of functional groups comprising the charged molecule (C) may be dependent or independent of the pH of the solution in which the charged molecule (C) is dispersed, such is the case, for example, for tertiary amines and quaternary ammonium compounds that are pH dependent and pH independent, respectively. The charge of a molecule can be readily estimated based on the molecule's Lewis structure and accepted methods known to those skilled in the art. Charge may result from inductive effects, e.g., atoms bonded together with differences in electron affinity may result in a polar covalent bond resulting in a partially negatively charged atom and a partially positively charged atom. For example, nitrogen bonded to hydrogen results in partial negative charge on nitrogen and a partial positive charge on the hydrogen atom. Alternatively, an atom in a molecule may be considered to have a full integer value of charge when the number of electrons assigned to that atom is less than or equal to the atomic number of the atom. The charge of the molecule is determined by summing the charge of each atom comprising the molecule. Those skilled in the art are familiar with the process of estimating charge of a molecule by summing the formal charge of each atom in a molecule.

The charged molecule (C) may either carry a net negative, net positive or neutral charge and depends on the net charge of the amphiphilic block copolymer needed for the specific application of the invention disclosed herein. For example, most cell surfaces are known to carry a net negative charge. Thus, net positively charged particles may interact with all cell surfaces without a high degree of specificity. In contrast, net negatively charged particles will be electrostatically repulsed from most cell surfaces but have been shown to promote selective uptake by certain antigen-presenting cell populations. For example, positively charged particles delivered intravenously into the circulation have been found to accumulate in the liver and lungs as well as within antigen-presenting cells in the spleen, whereas negatively charged particles have been found to preferentially accumulate in antigen-presenting cells in the spleen following intravenous administration. Thus, the net charge of the charged molecule (C) can be adjusted to meet the specific demands of the application.

In some embodiments, the surface stabilizing group (S) comprises a charged molecule (C) that has a net negative charge and is comprised of functional groups that carry a negative charge at physiologic pH, at a pH of about 7.4. Suitable charged molecules (C) that carry a net negative charge include molecules bearing functional groups (e.g., functional groups with a pKa less than about 6.5) that occur as the conjugate base of an acid at physiologic pH, at a pH of about 7.4. These include but are not limited to molecules bearing carboxylates, sulfates, phosphates, phosphoramidates, and phosphonates. The charged molecule (C) bearing a carboxylate can be but is not limited to glutamic acid, aspartic acid, pyruvic acid, lactic acid, glycolic acid, glucuronic acid, citrate, isocitrate, alpha-keto-glutarate, succinate, fumarate, malate, and oxaloacetate and derivatives thereof. In certain embodiments, the negatively charged molecule (C) is comprised of a molecule with between 1-20 negatively charged functional groups, such as 1, 2, 3, 4, 5, 6, 7, 8, 9, 10, 11, 12, 13, 14, 15, 16, 17, 18, 19 or 20 negatively charged functional groups, though, typically no more than 16 negatively charged functional groups. In some embodiments, the charged molecule (C) is a poly(glutamic acid) peptide of between 2-6 amino acids in length. A poly(glutamic acid) sequence comprised of 1, 2, 3, 4, 5 or 6 amino acids would be expected to carry a negative charge of −1, −2, −3, −4, −5 and −6 at pH 7.4, respectively. In additional embodiments, the charged molecule (C) is phosphoserine or sulfoserine.

In certain embodiments, the surface stabilizing group (S) comprises a charged molecule (C) that has a net negative charge and is comprised of 1 or more negatively charged amino acids. In certain embodiments, the charged molecule (C) with a net negative charge is comprised of between 1 to 20 negatively charged amino acids, e.g., 1, 2, 3, 4, 5, 6, 7, 8, 9, 10, 11, 12, 13, 14, 15, 16, 17, 18, 19 or 20. In a non-limiting example, a charged molecule (C) is comprised of 16 aspartic acid monomers, e.g., Asp-Asp-Asp-Asp-Asp-Asp-Asp-Asp-Asp-Asp-Asp-Asp-Asp-Asp-Asp-Asp (SEQ ID NO:1), is used to prepare a charged molecule (C) with a net negative charge of −16; a charged molecule (C) comprised of 15 aspartic acid monomers, e.g., Asp-Asp-Asp-Asp-Asp-Asp-Asp-Asp-Asp-Asp-Asp-Asp-Asp-Asp-Asp (SEQ ID NO:2), is used to prepare a charged molecule (C) with a net negative charge of −15; a charged molecule (C) comprised of 14 aspartic acid monomers, e.g., Asp-Asp-Asp-Asp-Asp-Asp-Asp-Asp-Asp-Asp-Asp-Asp-Asp-Asp (SEQ ID NO:3), is used to prepare a charged molecule (C) with a net negative charge of −14; a charged molecule (C) comprised of 13 aspartic acid monomers, e.g., Asp-Asp-Asp-Asp-Asp-Asp-Asp-Asp-Asp-Asp-Asp-Asp-Asp (SEQ ID NO:4), is used to prepare a charged molecule (C) with a net negative charge of −13; a charged molecule (C) comprised of 12 aspartic acid monomers, e.g., Asp-Asp-Asp-Asp-Asp-Asp-Asp-Asp-Asp-Asp-Asp-Asp (SEQ ID NO:5), is used to prepare a charged molecule (C) with a net negative charge of −12; a charged molecule (C) comprised of 11 aspartic acid monomers, e.g., Asp-Asp-Asp-Asp-Asp-Asp-Asp-Asp-Asp-Asp-Asp (SEQ ID NO:6), is used to prepare a charged molecule (C) with a net negative charge of −11; a charged molecule (C) comprised of 10 aspartic acid monomers, e.g., Asp-Asp-Asp-Asp-Asp-Asp-Asp-Asp-Asp-Asp (SEQ ID NO:7), is used to prepare a charged molecule (C) with a net negative charge of −10; a charged molecule (C) comprised of 9 aspartic acid monomers, e.g., Asp-Asp-Asp-Asp-Asp-Asp-Asp-Asp-Asp (SEQ ID NO:8), is used to prepare a charged molecule (C) with a net negative charge of −9; a charged molecule (C) comprised of 8 aspartic acid monomers, e.g., Asp-Asp-Asp-Asp-Asp-Asp-Asp-Asp (SEQ ID NO:9), is used to prepare a charged molecule (C) with a net negative charge of −8; a charged molecule (C) comprised of 7 aspartic acid monomers, e.g., Asp-Asp-Asp-Asp-Asp-Asp-Asp (SEQ ID NO: 10), is used to prepare a charged molecule (C) with a net negative charge of −7; a charged molecule (C) comprised of 6 aspartic acid monomers, e.g., Asp-Asp-Asp-Asp-Asp-Asp (SEQ ID NO:11), is used to prepare a charged molecule (C) with a net negative charge of −6; a charged molecule (C) comprised of 5 aspartic acid monomers, e.g., Asp-Asp-Asp-Asp-Asp (SEQ ID NO: 12), is used to prepare a charged molecule (C) with a net negative charge of −5; a charged molecule (C) comprised of 4 aspartic acid monomers, e.g., Asp-Asp-Asp-Asp (SEQ ID NO:13), is used to prepare a charged molecule (C) with a net negative charge of −4; a charged molecule (C) comprised of 3 aspartic acid monomers, e.g., Asp-Asp-Asp, is used to prepare a charged molecule (C) with a net negative charge of −3; a charged molecule (C) comprised of 2 aspartic acid monomers, e.g., Asp-Asp, is used to prepare a charged molecule (C) with a net negative charge of −2; a charged molecule (C) comprised of 1 aspartic acid monomer, e.g., Asp, is used to prepare a charged molecule (C) with a net negative charge of −1. In the above examples, aspartic acid (Asp) may be replaced with any suitable negatively charged amino acid, including but not limited to glutamic acid, sulfo-serine, or phosphor-serine, wherein the negatively charged amino acids may be the same or different.

In some embodiments, the surface stabilizing group (S) comprises a charged molecule (C) that has a net positive charge and is comprised of positively charged functional groups. Suitable positively charged molecules (C) include those with functional groups that carry positive charge at physiologic pH, at a pH of about 7.4, such as the conjugate acid of weak bases, wherein the pKa of the conjugate acid of the base is greater than about 8.5. Suitable positively charged molecules (C) include but are not limited to molecules bearing primary, secondary and tertiary amines, as well as quaternary ammonium, guanidinium, phosphonium and sulfonium functional groups. Suitable molecules bearing ammonium functional groups include, for example, imidazolium, and tetra-alkyl ammonium compounds. In some embodiments, the charged molecule (C) is comprised of quaternary ammonium compounds that carry a permanent positive charge that is independent of pH.

Non-limiting examples of positively charged functional groups that have charge independent of pH include:

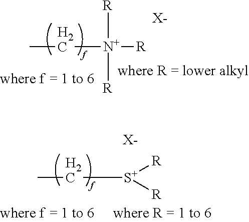

wherein X⁻ is any suitable counter anion.

In additional embodiments, the surface stabilizing group (S) comprises a charged molecule (C) that is comprised of functional groups that occur as the conjugate acid of a base at physiologic pH, such as, for example, primary, secondary and tertiary amines. In certain embodiments, the positively charged molecule (C) is comprised of between 1-20 positively charged functional groups, such as 1, 2, 3, 4, 5, 6, 7, 8, 9, 10, 11, 12, 13, 14, 15, 16, 17, 18, 19 or 20 positively charged functional groups, though, typically no more than 16 charged functional groups. In some embodiments, the charged molecule (C) is a poly(lysine) peptide of between 1-6 amino acids in length. A poly(lysine) sequence comprised of 1, 2, 3, 4, 5 or 6 amino acids would be expected to carry a positive charge of +1, +2, +3, +4, +5 or +6 respectively, at pH 7.4. In additional embodiments, the charged molecule (C) is a poly(arginine) peptide of between 2-6 amino acids in length.

In certain embodiments, the surface stabilizing group (S) comprises a charged molecule (C) that has a net positive charge and is comprised of 1 or more positively charged amino acids. In certain embodiments, the charged molecule (C) with a net positive charge is comprised of between 1 to 20 positively charged amino acids, e.g., 1, 2, 3, 4, 5, 6, 7, 8, 9, 10, 11, 12, 13, 14, 15, 16, 17, 18, 19 or 20. In a non-limiting example, a charged molecule (C) comprised of 16 lysine monomers, e.g., Lys-Lys-Lys-Lys-Lys-Lys-Lys-Lys-Lys-Lys-Lys-Lys-Lys-Lys-Lys-Lys (SEQ ID NO:14), is used to prepare a charged molecule (C) with a net positive charge of +16; a charged molecule (C) comprised of 15 lysine monomers, e.g., Lys-Lys-Lys-Lys-Lys-Lys-Lys-Lys-Lys-Lys-Lys-Lys-Lys-Lys-Lys (SEQ ID NO:15), is used to prepare a charged molecule (C) with a net positive charge of +15; a charged molecule (C) comprised of 14 lysine monomers, e.g., Lys-Lys-Lys-Lys-Lys-Lys-Lys-Lys-Lys-Lys-Lys-Lys-Lys-Lys (SEQ ID NO:16), is used to prepare a charged molecule (C) with a net positive charge of +14; a charged molecule (C) comprised of 13 lysine monomers, e.g., Lys-Lys-Lys-Lys-Lys-Lys-Lys-Lys-Lys-Lys-Lys-Lys-Lys (SEQ ID NO:17), is used to prepare a charged molecule (C) with a net positive charge of +13; a charged molecule (C) comprised of 12 lysine monomers, e.g., Lys-Lys-Lys-Lys-Lys-Lys-Lys-Lys-Lys-Lys-Lys-Lys (SEQ ID NO:18), is used to prepare a charged molecule (C) with a net positive charge of +12; a charged molecule (C) comprised of 11 lysine monomers, e.g., Lys-Lys-Lys-Lys-Lys-Lys-Lys-Lys-Lys-Lys-Lys (SEQ ID NO:19), is used to prepare a charged molecule (C) with a net positive charge of +11; a charged molecule (C) comprised of 10 lysine monomers, e.g., Lys-Lys-Lys-Lys-Lys-Lys-Lys-Lys-Lys-Lys (SEQ ID NO:20), is used to prepare a charged molecule (C) with a net positive charge of +10; a charged molecule (C) comprised of 9 lysine monomers, e.g., Lys-Lys-Lys-Lys-Lys-Lys-Lys-Lys-Lys (SEQ ID NO:21), is used to prepare a charged molecule (C) with a net positive charge of +9; a charged molecule (C) comprised of 8 lysine monomers, e.g., Lys-Lys-Lys-Lys-Lys-Lys-Lys-Lys (SEQ ID NO:22), is used to prepare a charged molecule (C) with a net positive charge of +8; a charged molecule (C) comprised of 7 lysine monomers, e.g., Lys-Lys-Lys-Lys-Lys-Lys-Lys (SEQ ID NO:23), is used to prepare a charged molecule (C) with a net positive charge of +7; a charged molecule (C) comprised of 6 lysine monomers, e.g., Lys-Lys-Lys-Lys-Lys-Lys (SEQ ID NO:24), is used to prepare a charged molecule (C) with a net positive charge of +6; a charged molecule (C) comprised of 5 lysine monomers, e.g., Lys-Lys-Lys-Lys-Lys (SEQ ID NO:25), is used to prepare a charged molecule (C) with a net positive charge of +5; a charged molecule (C) comprised of 4 lysine monomers, e.g., Lys-Lys-Lys-Lys (SEQ ID NO:26), is used to prepare a charged molecule (C) with a net positive charge of +4; a charged molecule (C) comprised of 3 lysine monomers, e.g., Lys-Lys-Lys, is used to prepare a charged molecule (C) with a net positive charge of +3; a charged molecule (C) comprised of 2 lysine monomers, e.g., Lys-Lys, is used to prepare a charged molecule (C) with a net positive charge of +2; a charged molecule (C) comprised of 1 lysine, e.g., Lys, is used to prepare a charged molecule (C) with a net positive charge of +1. In the above examples, Lysine (Lys) may be replaced with any suitable positively charged amino acid, including but not limited to trimethyl-lysine or arginine, wherein the positively charged amino acids may be the same or different.

The surface stabilizing group (S) comprising a charged molecule (C) may additionally comprise small non-charged, hydrophilic amino acids, or hydrophilic linkers, e.g., ethylene oxide that function to i) improve water solubility and ii) increase the distance between charged functional groups to prevent incomplete ionization. For instance, ionization of one functional group on a polymer may impact the pKa of neighboring functional groups through local effects. For example, protonation of an amine in close proximity to a second amine may lower the pKa of the conjugate acid of the second amine. To reduce the impact of local effects on the ionization potential of neighboring functional groups, a linker molecule may be used to increase the distance between charged functional groups comprising the charged molecule. The linker molecule may comprise between 1-5 small, non-charged hydrophilic amino acids, e.g., 1, 2, 3, 4, and 5 amino acids. Alternatively, the linker may comprise an ethylene oxide (i.e. PEG) linker between 1-4 monomers units, e.g., 1, 2, 3, or 4 ethylene oxide monomers in length. In certain embodiments, 1 to 2 small, non-charged hydrophilic amino acids are placed between neighboring charged amino acids comprising the charged molecule (C), wherein the amino acids are linked through amide bonds. In certain embodiments, a serine is placed between each charged amino acid comprising a charged molecule (C) with a net positive charge

In additional embodiments, the surface stabilizing group (S) comprises a charged molecule (C) comprised of both negatively and positively charged amino acids. Di-peptides comprised of amino acids of opposite charge, e.g., Lys-Asp, are referred to as zwitterion dipeptides because they are predicted to have a net neutral, 0, charge at pH 7.4. One or more zwitterion dipeptides can be included in the charged molecule (C) as a means to i) improve water solubility and ii) provide a prevailing charge (e.g., net negative or net positive) over certain pH ranges. For instance, a zwitterion di-peptide can be used to increase the hydrophilic character of a peptide sequence without increasing or decreasing the charge of a peptide sequence at pH 7.4. However, the zwitterion can be used to impart a net charge at a particular pH. For instance, excluding the contribution of the N-terminal amine and the C-terminal carboxylic acid in this example, the zwitterion di-peptide, Lys-Asp, has a net charge of 0 at pH 7.4, but a net charge of +1 at pH <4 and a net charge of −1 at pH>10. One or more zwitterion di-peptides can be added to the sequence of charged molecules (C); for example, one di-peptide, Lys-Asp; two di-peptides Lys-Asp-Lys-Asp (SEQ ID NO:27); three di-peptides, Lys-Asp-Lys-Asp-Lys-Asp (SEQ ID NO:28) and so forth. In the above examples, Lysine (Lys) may be replaced with any suitable positively charged amino acid, including but not limited to trimethyl-lysine or arginine, and aspartic acid (Asp) may be replaced with any suitable negatively charged amino acid, including but not limited to glutamic acid, sulfo-serine, or phospho-serine, wherein the positively or negatively charged amino acids may be the same or different.

The composition of the surface stabilizing group (S) comprising a charged molecule (C) is selected to provide the net charge needed for the specific application. In several embodiments disclosed herein, the charged molecule (C) is a positively charged poly(amino acid) comprised of lysines or arginines, or lysines or arginines and non-charged amino acids. In some embodiments the charged molecule (C) comprises sulfonium or quaternary ammonium functional groups that carry pH independent positive charge. In several embodiments disclosed herein, the charged molecule (C) is a negatively charged poly(amino acid) comprised of glutamic acid or aspartic acid, or glutamic acid or aspartic acid and non-charged amino acids. In some embodiments the charged moiety comprises phosphate or sulfate groups, such as sulfoserine or phosphoserine. In additional embodiments, the charged molecule is comprised of lysines or arginines and glutamic acid or aspartic acid, or lysines or arginines and glutamic acid or aspartic acid as well as non-charged amino acids. Both positive and negatively charged functional groups may be included on the same charged molecule (C). The charged molecule (C) may be positive, negative or neutral. In preferred embodiments of amphiphilic block copolymers comprised of S-[B] with linear architecture (i.e. non-brush or cone)S-[B], the net charge is preferably non-zero, for example, greater than +3 or less than −3 net charge, typically greater than +4 or less than −4 net charge, to ensure that sufficient charge is provided to prevent aggregation.

An unexpected finding reported herein is that amphiphilic block copolymers comprised of S-[B] with brush architecture, i.e. two or more S-[B] groups linked to an amplifying linker that is linked to a hydrophobic polymer or oligomer (H), required less net charge than those with linear architecture. Therefore, in preferred embodiments of amphiphilic block copolymers comprised of S-[B] with brush architecture, the net charge is preferably neutral, for example, between about +4 to −4, such as +4, +3, +2, +1, 0, −1, −2, −3, −4, typically neutral, i.e. o, charge.

In some embodiments the surface stabilizing group (S) comprising a charged molecule is comprised of nucleic acids. In some embodiments, the nucleic acid is therapeutically active, such as an siRNA sequence or a nucleic acid that binds pattern recognition receptors (PRRs). In some embodiments the surface stabilizing group is an oligo-deoxynucleotide that binds to Toll-like receptor 9.

An additional consideration regarding charged molecules (C) is the counterion selected. Non-limiting examples of charged molecules (C) bearing functional groups with positive charge include but are not limited to halides, including chloride, bromide and iodide anions, and conjugate bases of acids, including, phosphate, sulfates, sulfites and carboxylate anions including formate, succinate, acetate and trifluoroacetate. Suitable counterions for charged molecules (C) bearing functional groups with negative charge include but are not limited to hydrogen and alkali and alkaline earth metals, including, for example, sodium, potassium, magnesium and calcium, or conjugate acids of weak bases, such as ammonium compounds. Suitable amines used to form the ammonium salt include but are not limited to ammonium, primary amines, such as tris(hydroxymethyl)aminomethane, secondary amines based on di-alkyl amines, such as dimethyl amine and diethyl amine, tertiary amines based on tri-alkyl amines, such as trimethylamine, di-isopropryl ethylamine (DIPEA) and triethylamine (TEA), as well as quaternary ammonium compounds. Unexpectedly, tris(hydroxymethyl)aminomethane (or Tris) as the ammonium salt of acids as the counterion of amphiphilic block copolymers with negative charge has improved solubility in both water-miscible organic solvents, such as DMSO, DMF, acetone and ethanol, and aqueous solutions. For these reasons, the protonated form of tris(hydroxymethyl)aminomethane is a preferred counter-ion to use in the preparation of salts of conjugate bases of acids present on the amphiphilic block copolymers of the present disclosure.

In certain embodiments, the surface stabilizing (S) group comprises hydrophilic polymers, e.g., synthetic polymers that comprise hydrophilic monomers selected from acrylates, meth(acrylates), e.g., hydroxyethylmethacrylate (HEMA), acrylamides, meth(acrylamides), e.g., N-(2-hydroxypropyl(methacrylamide)) (HPMA), ethylene oxide. In other embodiments, the surface stabilizing (S) group comprises hydrophilic polymers selected from synthetic or natural poly(saccharides), such as glycogen, cellulose, dextran, alginate and chitosan. The hydrophilic polymers (H) typically comprise between 30-300 monomers and have a molecular weight between about 1,000 to about 60,000 g/mol. Hydrophilic polymers used as the surface stabilizing group (S) should have sufficient length to provide adequate surface coverage to stabilize particles formed by the amphiphilic block copolymers described herein. Unexpectedly, it was found that amphiphilic block copolymers comprised of neutral hydrophilic polymer-based surface stabilizing groups (S) with greater than 50 monomer units formed stable nanoparticle micelles, whereas those with fewer monomers tended to aggregate. Therefore, in preferred embodiments, the amphiphilic block copolymers comprised of neutral hydrophilic polymer-based surface stabilizing groups (S) have 50 or monomer units, such as between 50 to 300, though, preferably between 50 and 100.

In certain embodiments, the hydrophilic surface stabilizing group (S) comprises one or more mono-saccharide or oligo-saccharide molecules. The present inventors have surprisingly found that S groups comprising mono-saccharide or oligo-saccharide molecules can stabilize the nanoparticle micelles and promote targeted delivery to specific cell subsets through C-type lectin receptors.

In some embodiments, the surface stabilizing group (S) is neutral, e.g., comprises one or more hydroxyl groups. An unexpected finding reported herein is that linear amphiphilic block copolymers typically require net charge to form stable nanoparticle micelles whereas amphiphilic block copolymers with brush architecture form stable nanoparticle micelles at neutral or near neutral net charge. Therefore, preferred embodiments of neutral amphiphilic block polymers have brush architecture and comprise surface stabilizing groups comprised of one or more hydroxyl groups. In some embodiments, the neutral amphiphilic block polymer has brush architecture and comprises surface stabilizing groups comprised of one or more saccharides, such as one or more monosaccharides, disaccharides, trisaccharides, tetrasaccharides or higher glycans.

In some embodiments, the surface stabilizing group (S) is linked to the hydrophobic polymer or oligomer (H) either directly or indirectly through a spacer molecule (B) and/or linker molecule.

In certain embodiments, the spacer molecule (B) comprises PEG and or peptide linker molecules. The present inventors have surprisingly found compositions of B that promote stable nanoparticle micelles and/or allow for efficient drug molecule (D) release.

The optional spacer (B) may be comprised of any one or more of the following: amino acids, including non-natural amino acids; hydrophilic polymers, e.g., polymers based on ethylene oxide (e.g., PEG) or methacrylate or methacrylamide based monomers; hydrophobic alkane chains; or the like; or combinations thereof. The spacer (B) may be linked to the surface stabilizing group (S) and hydrophobic polymer or oligomer (H) through any suitable means, e.g., through stable amide bonds. While spacer groups (B) and surface stabilizing groups (S) may both be comprised of hydrophilic polymers (e.g., hydrophilic poly(amino acids); hydrophilic methacrylate-based polymers, such as HEMA; hydrophilic methacrylamide-based polymers, such as HPMA and/or PEG (comprised of ethylene oxide monomers)), the distinction between S and B is based on function.

In some embodiments, the spacer (B) functions to provide distance, i.e. space, between the heterologous molecules, S and H. In other embodiments, the spacer (B) functions to impart hydrophobic or hydrophilic properties to the block copolymer. In still other embodiments, the composition of the spacer may be selected to impart rigidity or flexibility. In other embodiments, the composition of the spacer may be selected for recognition by enzymes and promote degradation of the block copolymer.

In some embodiments, the spacer (B) is a peptide sequence between about 1 to 30 amino acids in length, such as 1, 2, 3, 4, 5, 6, 7, 8, 9, 10, 15, 20, 25 or 30 amino acids, typically no more than 30 amino acids in length, that is linked to the hydrophobic polymer or oligomer (H) and surface stabilizing group (S) through, e.g., an amide bond formed between the N- or C-terminal carboxyl group of the spacer (B). The amide bond between the spacer (B) and the surface stabilizing group (S) and/or H may be recognized by enzymes or may be selected for resistance to enzyme-mediated hydrolysis.

In some embodiments, the spacer (B) is a hydrophilic polymer, such as PEG, HPMA or HEMA, and is between about 1 to 30 monomers in length, such as 1, 2, 3, 4, 5, 6, 7, 8, 9, 10, 15, 20, 25 or 30 monomers, typically no more than 30 monomers in length, that is linked to the hydrophobic polymer or oligomer (H) and surface stabilizing group (S) either directly or through linkers.

Linkers generally refer to any molecules that join together any two or more heterologous molecules. The linker may use covalent or non-covalent means to join any two or more components, i.e. heterologous molecules, for example a surface stabilizing group (S) and a hydrophobic polymer or oligomer (H).

In certain embodiments, a linker may join, i.e. link, any two components of the amphiphilic block copolymer through a covalent bond. Covalent bonds are the preferred linkages used to join any two components, i.e. heterologous molecules, of the amphiphilic block copolymer and ensure that no component is able to immediately disperse from the other components following administration to a subject.

The linkage used to join any two heterologous molecules may comprise any suitable functional group, including but not limited to amides, esters, ethers, thioethers, disulfides, carbamates, carbamide, hydrazides, hydrazones, acetals and triazoles. In a non-limiting example of a covalent linkage, a click chemistry reaction may result in a triazole that links, i.e. joins together, any two components of the amphiphilic block copolymer. In several embodiments, the click chemistry reaction is a strain-promoted [3+2] azide-alkyne cyclo-addition reaction. An alkyne group and an azide group may be provided on respective molecules comprising the amphiphilic block copolymer to be linked by “click chemistry”. In some embodiments, a drug molecule (D), such as a TLR agonist, bearing an azide functional group is coupled to a hydrophobic polymer or oligomer (H) having an appropriate reactive group, such as an alkyne, for example, a dibenzylcyclooctyne (DBCO).

In preferred embodiments, the amphiphilic block copolymer is comprised of poly(amino acid)-based S, B and H blocks that are linked together through amide bonds. For example, each amino acid comprising S, B and H may be linked together sequentially by reacting a carboxylic acid (or activated carboxylic acid) of one monomer with an amine of another monomer to form an amide bond. Alternatively, pre-formed poly(amino acid) blocks of S, B and H (or S-B and H, or S and B-H), which may each be prepared by solid phase peptide synthesis, may be joined together by reacting a carboxylic acid (or activated carboxylic acid) of one block with an amine of another block to form an amide bond that joins together the blocks. In some embodiments, the amphiphilic block copolymer is comprised of poly(amino acid)-based S, B and H blocks that are joined together through amide bonds during solid phase peptide synthesis. In some embodiments, the amphiphilic block copolymer is comprised of poly(amino acid)-based S, B and H blocks that are joined together through amide bonds during solution phase synthesis. In other embodiments, the amphiphilic block copolymer is comprised of poly(amino acid)-based S, B and H blocks that are joined together through amide bonds during solid phase peptide synthesis and/or solution phase synthesis. Note: the spacer molecule (B) is optional in the examples provided in this paragraph.

In some embodiments, the blocks S, B and H, or S-B and H, or S and B-H may be joined by reacting a reactive precursor X1 on one block with a reactive precursor X2 on the other block to form a linkage. In some embodiments, any two or more blocks are linked together through an amide bond formed by reacting a linker precursor X1 that comprises an activated carboxylic acid with a linker precursor X2 that comprises an amine. In other embodiments, any two or more blocks are linked together through an amide bond formed by reacting a linker precursor X1 that comprises an amine with a linker precursor X2 that comprises an activated carboxylic acid. In some embodiments, any two or more blocks are linked together through a thioether bond formed by reacting a linker precursor X1 that comprises a maleimide with a linker precursor X2 that comprises a thiol. In other embodiments, any two or more blocks are linked together through a thioether bond formed by reacting a linker precursor X1 that comprises a thiol with a linker precursor X2 that comprises a maleimide. In some embodiments, any two or more blocks are linked together through a triazole group formed by reacting a linker precursor X1 that comprises an azide with a linker precursor X2 that comprises an alkyne. In other embodiments, any two or more blocks are linked together through a triazole group formed by reacting a linker precursor X1 that comprises an alkyne with a linker precursor X2 that comprises an azide. Note: the spacer molecule (B) is optional in the examples provided in this paragraph.

In preferred embodiments of amphiphilic block copolymers that comprise S-[B]-H joined together by linking S-B and H, S and H, or S and B-H, through a triazole bond, one block (e.g., H) comprises a linker precursor X1 that comprises a strained alkyne (e.g., dibenzocyclooctyne (DBCO), bicyclononyne (BCN) or the like) is linked to a another block (e.g., S-[B]) that comprises a linker precursor X2 that comprises an azide to form a triazole group that joins S-[B] and H to form the amphiphilic block copolymer S-[B]-H. In a non-limiting example, a DBCO based linker precursor X1 is linked to a poly(amino acid) based hydrophobic polymer or oligomer (H) at the N-terminus through any suitable means (e.g., DBCO-NHS, CAS number 1353016-71-3) and an azide-based linker precursor X2 (e.g. azido acid, such as azidopentanoic acid; azido amino acid, such as azido-lysine (abbreviated Lys(N3), CAS number 159610-92-1); or azido amine, such as azido-butylamine) is linked to a poly(amino acid) and/or hydrophilic polymer based S-[B] through any suitable means; the X1 linker precursor bearing the strained alkyne, i.e. DBCO, is reacted with the linker precursor X2 bearing an azide resulting in a triazole group to form S-[B]-H. Note: the spacer molecule (B) is optional in the examples provided in this paragraph.

There are many suitable linkers that are well known to those of skill in the art and include, but are not limited to, straight or branched-chain carbon linkers, heterocyclic carbon linkers, rigid aromatic linkers, flexible ethylene oxide linkers, peptide linkers, or a combination thereof. In some embodiments, the carbon linker can include a C1-C18 alkane linker, such as a lower alkyl C4; the alkane linkers can serve to increase the space between two or more heterologous molecules, while longer chain alkane linkers can be used to impart hydrophobic characteristics. Alternatively, hydrophilic linkers, such as ethylene oxide linkers, may be used in place of alkane linkers to increase the space between any two or more heterologous molecules and increase water solubility. In other embodiments, the linker can be an aromatic compound, or poly(aromatic) compound that imparts rigidity. The linker molecule may comprise a hydrophilic or hydrophobic linker. In several embodiments, the linker includes a degradable peptide sequence that is cleavable by an intracellular enzyme (such as a cathepsin or the immuno-proteasome).

An unexpected finding disclosed herein is that the reaction rate for attachment of different molecules, e.g., S-B, B, linker molecules or linker precursors (e.g., DBCO based X1), to the N-terminal amine of poly(amino acid)-based hydrophobic polymers (H) can be increased by increasing the number of methylene units between the amide and the amine of the N-terminal amino acid. Importantly, these findings are not limited to the reactivity of the N-terminal amino acid of poly(amino acid)-based hydrophobic polymers and suggest that amino acid-based linkers, whenever possible, should comprise two or more methylene units, to improve reactivity. Therefore, in preferred embodiments, the N-terminal amino acid of peptide-based hydrophobic polymers comprise two or more, typically between 2 and 7, such as 1, 2, 3, 4, 5, 6, 7 methylene units. For clarity, an amino acid with 2 methylene units is beta-alanine and an amino acid with 5 methylene units is amino-hexanoic acid. In preferred embodiments, the N-terminal amino acid of peptide-based hydrophobic polymer is amino-hexanoic acid (sometimes referred to as Ahx; CAS number 60-32-3). In other embodiments, the N-terminal amino acid of peptide-based hydrophobic polymers is beta-alanine.

In some embodiments, the linker may be comprised of poly(ethylene oxide) (PEG). The length of the linker depends on the purpose of the linker. For example, the length of the linker, such as a PEG linker, can be increased to separate components of the amphiphilic block copolymer, for example, to reduce steric hindrance, or in the case of a hydrophilic PEG linker can be used to improve water solubility. The linker, such as PEG, may be a short linker that may be at least 2 monomers in length. The linker, such as PEG, may be between about 4 and about 24 monomers in length, such as 4, 5, 6, 7, 8, 9, 10, 11, 12, 13, 14, 15, 16, 17, 18, 19, 20, 21, 22, 23, 24 monomers in length or more. In some embodiments, a Ligand is linked to a hydrophobic polymer or oligomer (H) though a PEG linker.

In some embodiments, where the linker comprises a carbon chain, the linker may comprise a chain of between about 1 or 2 and about 18 carbons, such as 1, 2, 3, 4, 5, 6, 7, 8, 9, 10, 11, 12, 13, 14, 15, 16, 17, 18 carbons in length or more. In some embodiments, where the linker comprises a carbon chain, the linker may comprise a chain of between about 12 and about 20 carbons. In some embodiments, where the linker comprises a carbon chain, the linker may comprise a chain of between no more than 18 carbons.

In some embodiments, the linker is cleavable under intracellular conditions, such that cleavage of the linker results in the release of any component linked to the linker, for example, a drug molecule (D).

For example, the linker can be cleavable by enzymes localized in intracellular vesicles (for example, within a lysosome or endosome or caveolae) or by enzymes, in the cytosol, such as the proteasome, or immuno-proteasome. The linker can be, for example, a peptide linker that is cleaved by protease enzymes, including, but not limited to proteases that are localized in intracellular vesicles, such as cathepsins in the lysosomal or endosomal compartment. The peptide linker is typically between 1-10 amino acids, such as 1, 2, 3, 4, 5, 6, 7, 8, 9, 10, or more (such as up to 20) amino acids long, such as 11, 12, 13, 14, 15, 16, 17, 18, 19, 20 or more amino acids long. Certain dipeptides are known to be hydrolyzed by proteases that include cathepsins, such as cathepsins B and D and plasmin, (see, for example, Dubowchik and Walker, 1999, Pharm. Therapeutics 83:67-123). For example, a peptide linker that is cleavable by the thiol-dependent protease cathepsin-B, can be used (for example, a Phe-Leu or a Gly-Phe-Leu-Gly (SEQ ID NO:107) linker). Other examples of such linkers are described, for example, in U.S. Pat. No. 6,214,345, incorporated herein by reference. In a specific embodiment, the peptide linker cleavable by an intracellular protease is a Val-Cit linker or a Phe-Lys linker (see, for example, U.S. Pat. No. 6,214,345, which describes the synthesis of doxorubicin with the Val-Cit linker).

The cleavable peptide linker can be selected to promote processing (i.e. hydrolysis) of the peptide linker following intracellular uptake by immune cells. The sequence of the cleavable peptide linker can be selected to promote processing by intracellular proteases, such as cathepsins in intracellular vesicles or the proteasome or immuno-proteasome in the cytosolic space.

In several embodiments, linkers comprised of peptide sequences of the formula Pn . . . P4-P3-P2-P1 are used to promote recognition by cathepsins, wherein P1 is selected from arginine, lysine, citrulline, glutamine, threonine, leucine, norleucine, or methionine; P2 is selected from glycine, leucine, valine or isoleucine; P3 is selected rom glycine, serine, alanine, proline or leucine; and P4 is selected from glycine, serine, arginine, lysine aspartic acid or glutamic acid. In a non-limiting example, a tetrapeptide linker of the formula P4-P3-P2-P1 linked through an amide bond to a heterologous molecule and has the sequence Lys-Pro-Leu-Arg (SEQ ID NO:29). For clarity, the amino acid residues (Pn) are numbered from proximal to distal from the site of cleavage, which is C-terminal to the P1 residue, for example, the amide bond between P1-P1′ is hydrolyzed. Suitable peptide sequences that promote cleavage by endosomal and lysosomal proteases, such as cathepsin, are well described in the literature (see: Choe, et al., J. Biol. Chem., 281:12824-12832, 2006).

In several embodiments, linkers comprised of peptide sequences are selected to promote recognition by the proteasome or immuno-proteasome. Peptide sequences of the formula Pn . . . P4-P3-P2-P1 are selected to promote recognition by proteasome or immuno-proteasome, wherein P1 is selected from basic residues and hydrophobic, branched residues, such as arginine, lysine, leucine, isoleucine and valine; P2, P3 and P4 are optionally selected from leucine, isoleucine, valine, lysine and tyrosine. In a non-limiting example, a cleavable linker of the formula P4-P3-P2-P1 that is recognized by the proteasome is linked through an amide bond at P1 to a heterologous molecule and has the sequence Tyr-Leu-Leu-Leu (SEQ ID NO:30). Sequences that promote degradation by the proteasome or immuno-proteasome may be used alone or in combination with cathepsin cleavable linkers. In some embodiments, amino acids that promote immuno-proteasome processing are linked to linkers that promote processing by endosomal proteases. A number of suitable sequences to promote cleavage by the immuno-proteasome are well described in the literature (see: Kloetzel, et al., Nat. Rev. Mol. Cell Biol., 2:179-187), 2001, Huber, et al., Cell, 148:727-738, 2012, and Harris et al., Chem. Biol., 8:1131-1141, 2001).

In some embodiments, the spacer (B) comprises an enzyme degradable peptide linker sequence.

In other embodiments, any two or more components of the amphiphilic block copolymer may be joined together through a pH-sensitive linker that is sensitive to hydrolysis under acidic conditions. A number of pH-sensitive linkages are familiar to those skilled in the art and include for example, a hydrazone, semicarbazone, thiosemicarbazone, cis-aconitic amide, orthoester, acetal, ketal, or the like (see, for example, U.S. Pat. Nos. 5,122,368; 5,824,805; 5,622,929; Dubowchik and Walker, 1999, Pharm. Therapeutics 83:67-123; Neville et al., 1989, Biol. Chem. 264:14653-14661). In certain embodiments, the linkage is stable at physiologic pH, e.g., at a pH of about 7.4, but undergoes hydrolysis at lysosomal pH, ˜pH 5-6.5. In some embodiments, a drug molecule (D), such as an anthracycline, is linked to a hydrophobic or oligomer (H) through a FG that forms a pH-sensitive bond, such as the reaction between a ketone and a hydrazine to form a pH labile hydrazone bond.

In other embodiments, the linker comprises a linkage that is cleavable under reducing conditions, such as a reducible disulfide bond. Many different linkers used to introduce disulfide linkages are known in the art (see, for example, Thorpe et al., 1987, Cancer Res. 47:5924-5931; Wawrzynczak et al., In Immunoconjugates: Antibody Conjugates in Radioimagery and Therapy of Cancer (C. W. Vogel ed., Oxford U. Press, 1987); Phillips et al., Cancer Res. 68:92809290, 2008). See also U.S. Pat. No. 4,880,935).

Particles comprising an amphiphilic block copolymer membrane and at least one drug molecule encapsulated therein, can be prepared by preparing an aqueous solution comprising said amphiphilic block copolymer under conditions to produce particles having the at least one drug molecule encapsulated therein.

The present inventors have found that dendritic amplifying linkers can be used to create cone or brush shaped amphiphilic block copolymers that form nanoparticle micelles with improved stability.

The present inventors have found that admixing one or more different compositions of molecules with an amphiphilic block copolymer of the present disclosure in an organic solvent such as DMSO and re-suspending in aqueous media results in the formation of stable nanoparticles (“mosaic particles”), such as nanoparticle micelles or polymersomes. The composition and architecture of the amphiphilic block copolymer can be used to control whether the particle is a micelle or polymersome, with amphiphilic block polymers typically assembling into micelles.

Surprisingly, the present inventors have found that mosaic particles comprising both amphiphilic block copolymers linked to a chemotherapeutic drug molecule (e.g., S-[B]-H(D)) and amphiphilic block copolymers linked to an immunostimulant drug molecule (e.g., (2)S-[B]-H(D)) lead to improved tumor clearance as compared with particles comprising either block copolymer drug conjugates alone.

Mosaic particles may comprise two or more different compositions of amphiphilic block copolymers, wherein the two or more different amphiphilic block copolymers may each optionally comprise one or more drug molecules, e.g., a mosaic particle comprising S-[B]-H+S-[B]-H(D), or S-[B]-H(D)+(2)S-[B]-H(D). Alternatively, mosaic particles may incorporate two or more different compositions of molecules each comprising a hydrophobic polymer or oligomer, wherein the two or more different molecules are selected from at least one amphiphilic block copolymers and one or more drug-hydrophobic polymer or oligomer conjugates, e.g., a mosaic particle comprising S-[B]-H+D-H.

In some embodiments, the mosaic particle comprises two or more different amphiphilic block copolymers and/or amphiphilic block copolymer drug conjugates of the formula S-[B]-H, S-[B]-H(D), S(D)-[B]-H, S-B(D)-H and D-[B]-H, optionally wherein one or more different drug molecules of the formula, D or D-[B]-H may be incorporated into the particle; each of the two or more different molecules of the same formula are proceeded by an integer value (1, 2, 3 . . . ) to distinguish different compositions of the same formula. Non-limiting examples of such particles include:
S-[B]-H+S-[B]-H(D)
S-[B]-H+D-[B]-H
(1)S-[B]-H+(2)S-[B]-H+D
(1)S-[B]-H+(2)S-[B]-H+D-H
(1)S-[B]-H+(2)S-[B]-H+(3)S-[B]-H+D-H

In some embodiments, the mosaic particle comprises an amphiphilic block copolymer of the formula S-[B]-H, S-[B]-H(D), S(D)-[B]-H, S-B(D)-H or D-[B]-H and two or more different drug molecules of the formula, D or D-[B]-H; each of the two or more different molecules of the same formula are proceeded by an integer value (1, 2, 3 . . . ) to distinguish different compositions of the same formula. Non-limiting examples of such particles include:
S-[B]-H+(1) D-[B]-H+(2) D-[B]-H
S-[B]-H+(2)S-[B]-H(D)+D
S-[B]-H+D-[B]-H+D-H
S-[B]-H+(2)S-[B]-H+(3)S-[B]-H+D-H

Selection of Counter-Ions for Charged Molecules (C) Based on Poly(Anions)

A challenge for producing charged molecules (C) comprised of anions based on acids, e.g., peptide-based oligomers or polymers based on aspartic acid, glutamic acid, sulfoserine or phosphoserine, is that the protonated forms of these acids are typically poorly soluble in aqueous solutions and often even have poor solubility in water-miscible organic solvents. The limited solubility of acids can create challenges to manufacturing and handling.

One means of improving manufacturing and handling of amphiphilic di-block copolymers, e.g., C-B-H comprised, wherein the charged molecule (C) comprises anions, is to select a suitable counter-ion.

While use of alkali metal ions, such as sodium (Na+) and potassium (K+), as the counter-ions of conjugate bases of acids provided salts (e.g., the sodium salt of carboxylate) with good water solubility, such salts were generally found to have insufficient solubility in water-miscible solvents, such as DMSO, DMF, methanol, ethanol and acetone, which are preferred solvent systems for solubilizing amphiphilic block copolymers prior to particle assembly.

In contrast, the conjugate acid of organic bases, such as those based on alkyl amines, particularly tri-alkyl amines, were found to improve solubility amphiphilic block copolymers in both water and water-miscible organic solvents. Therefore, in certain embodiments, amphiphilic block copolymers that comprise acids are prepared as the ammonium salt form of the acid. Suitable amines used to form the ammonium salt include but are not limited to ammonium, primary amines, such as tris(hydroxymethyl)aminomethane, secondary amines based on di-alkyl amines, such as dimethyl amine and diethyl amine, tertiary amines based on tri-alkyl amines, such as trimethylamine, di-isopropryl ethylamine (DIPEA) and triethylamine (TEA), as well as quaternary ammonium compounds. Unexpectedly, tris(hydroxymethyl)aminomethane (or Tris) as the ammonium salt of acids present on amphiphilic block copolymers improved solubility of such molecules in both water-miscible organic solvents, such as DMSO, DMF, acetone and ethanol, and aqueous solutions; additionally, the ammonium salts of amphiphilic block copolymers prepared from tris(hydroxymethyl)aminomethane had minimal impact on the pH of the aqueous buffer, such as PBS, pH 7.4, when such salts were suspended in aqueous buffers. For these reasons, the protonated form of tris(hydroxymethyl)aminomethane is a preferred counter-ion to use in the preparation of salts of conjugate bases of acids present on the amphiphilic block copolymers disclosed herein.

Number and Selection of Charged Functional Groups

The number of charged functional groups included in amphiphilic block copolymers is selected to ensure stable nanoparticle micelle formation and to prevent formation of aggregates. In some embodiments, 4 or more amine or guanidine functional groups are needed to ensure stable nanoparticle micelle formation with amphiphilic block copolymers of formula S-B-H. In other embodiments, 4 or more carboxylate functional groups are needed to ensure stable nanoparticle micelle formation with amphiphilic block copolymers of formula S-B-H. Unexpectedly, amphiphilic block copolymers of formula S-B-H were found to form stable nanoparticle micelles with as few as two or more functional groups comprised of sulfonates, sulfates, phosphonates and/or phosphates.

Moreover, the number of charged functional groups needed to ensure stable nanoparticle micelle formation was also found to be dependent on the composition of the spacer, B, and the architecture of the amphiphilic block copolymers.

Linear amphiphilic block copolymers of formula S-B-H, wherein B is comprised of small and/or hydrophilic amino acids and H is comprised of 5 or more hydrophobic amino acids, such as 5 or more Tryptophan amino acids, typically required charged molecules with a greater number of charged functional groups, such as 6 or more, sometimes 10 or more amines, guanidines and/or carboxylates, or 3 or more, sometimes 4 or more, sulfonates, sulfates, phosphonates and/or phosphates. In contrast, linear amphiphilic block copolymers of formula S-B-H, wherein B is comprised of a hydrophilic polymer, such as PEG or HPMA, and H is comprised of 5 or more hydrophobic amino acids, such as 5 or more Tryptophan amino acids, typically required charged molecules with fewer charged functional groups, such as 4 or more, sometimes 8 or more amines, guanidines and/or carboxylates, or 2 or more, sometimes 3 or more, sulfonates, sulfates, phosphonates and/or phosphates.

Additionally, the association between amphiphilic block copolymer net charge and nanoparticle micelle formation was also found to be strongly dependent on the architecture of the amphiphilic block copolymers. Notably, an unexpected finding reported herein is that amphiphilic block copolymers with brush architecture required fewer charged functional groups than those with linear architecture. Amphiphilic block copolymers with brush architecture may be prepared by linking hydrophobic polymers (H) to amplifying linkers that provide two or more attachment points for every C-B, for instance, (C-B)y19-K-H, wherein K is an amplifying linker and y19 denotes that there are an integer number, typically between 2 and 8, of C-B attached to the amplifying linker, which is attached either directly or through a linker to a hydrophobic polymer or oligomer (H).

A non-limiting example of an amphiphilic block copolymer with brush architecture of formula (C-B)y19-K-H, wherein y19 is 4, is provided here for clarity:

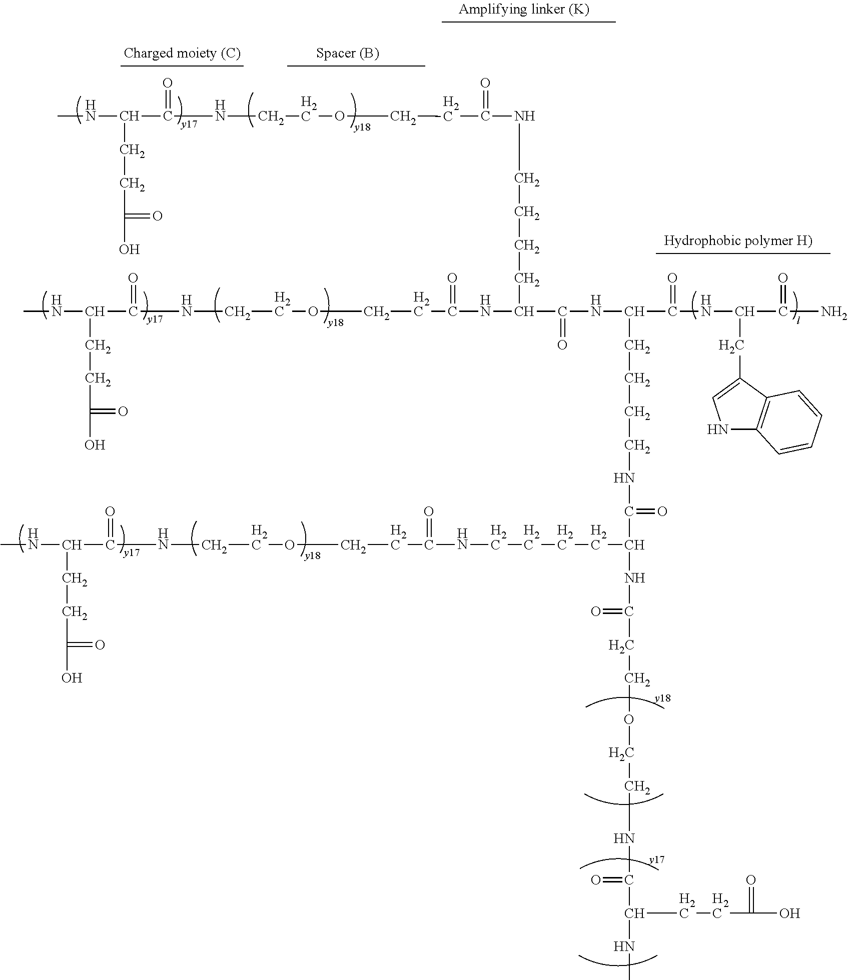

Wherein y17 is an integer number of repeating units of monomers comprising the charged molecule (C) (sometimes referred to as charged moiety), typically selected from between about 1 to 16, such as 1, 2, 3, 4, 5, 6, 7, 8, 9, 10, 11, 12, 13, 14, 15, 16; y18 is an integer number of repeating units of monomers comprising the spacer (B), which is typically between about 4 to about 30; the amine of the N-terminal amino acid of the peptide-based charged molecule (C), as shown in this example, is either in the form of the free amine or is capped, e.g., with an acyl group; and, the hydrophobic polymer (sometimes referred to as the hydrophobic block) is poly(tryptophan).

An additional non-limiting example of an amphiphilic block copolymer with brush architecture of formula (C-B)y19-K-H, wherein y19 is 4, is provided here for clarity:

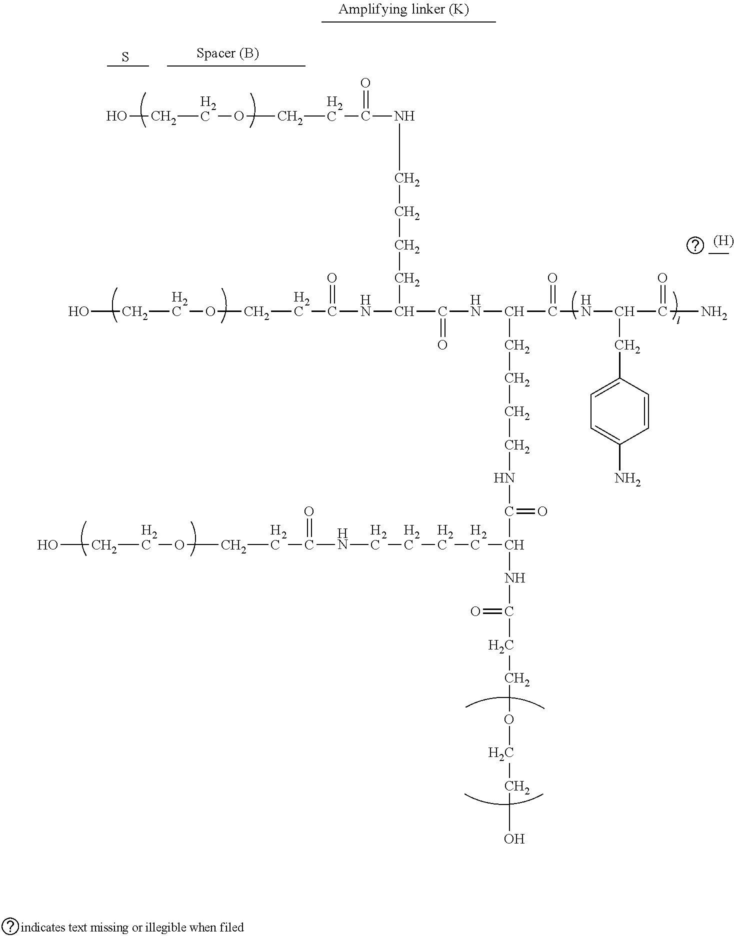

Wherein the surface stabilizing group is hydrophilic and comprises a hydroxyl group; y18 is an integer number of repeating units of monomers comprising the spacer (B), which is typically between about 4 to about 30; and, the hydrophobic polymer is poly(amino phenylalanine).

Placement of Amino Acids Bearing Aryl Amines on Peptide-Based Hydrophobic Polymers

Amino acids comprising a lower alkyl amine or guanidine carry positive charge at physiologic pH (˜pH 7.4) that helps to improve solubility in aqueous solutions at or near physiologic pH, but such properties, i.e. solubility at physiologic pH, may not be desirable when such amino acids are placed at or near the hydrophobic polymers (H). Therefore, the current challenge that the inventors of the present disclosure sought to address is the need to improve manufacturability of peptide-based hydrophobic polymers without adversely impacting the capacity of amphiphilic block copolymers based on such materials to form stable particles in aqueous solutions around physiologic pH.

Recognizing this challenge, the inventors of the present disclosure introduced two novel approaches to leverage the benefits of incorporating amino acids bearing amine functional groups at or near the hydrophobic polymers or oligomers (H) without adversely impacting the hydrophobic characteristics of the hydrophobic polymers or disrupting particle formation by the amphiphilic block copolymers described herein. One approach was to introduce alkyl amines into peptide-based hydrophobic polymers during manufacturing but to cap (e.g., acylate) the alkyl amine groups prior to their incorporation into amphiphilic block copolymers. Another approach was to incorporate amino acids bearing aryl amines, which carry a positive charge at pH below physiologic pH, e.g., pH less than 6.5, but are neutral (non-charged) at physiologic pH, into peptide sequences, e.g., peptide-based hydrophobic polymers.

An unexpected finding disclosed herein is that the incorporation of one or more amino acids, such as between 1 and 30, bearing an aryl amine functional group into peptides during solid-phase peptide synthesis led to improved manufacturability as compared with peptides lacking amino acids bearing the aryl amine functional group. These findings were unexpected as amino acids comprising aromatic groups are often considered difficult to manufacture owing to their hydrophobic characteristics. However, unexpectedly, as reported herein, addition of amino acids with aromatic amines (aryl amines) to peptide sequences led to improved manufacturability comparable to that observed with the addition of lower alkyl amines.

While amino acids bearing an aryl amine improved manufacturability, such amino acids are typically highly hydrophobic in aqueous conditions at physiologic pH (˜pH 7.4). Therefore, such amino acids should be placed at or near the hydrophobic polymer but preferably not placed at or near the charged molecule of amphiphilic block copolymers.

Selection of the Number of Amino Acids Bearing Aryl Amines

Unexpectedly, the inventors of the present disclosure found that between 1 and 10, such as 1, 2, 3, 4, 5, 6, 7, 8, 9 and 10 amino acids bearing aryl amine groups were sufficient to improve manufacturability and solubility of peptide-based hydrophobic polymers. Therefore, in preferred embodiments of peptide-based hydrophobic polymers, the number of amino acids bearing aryl amines is typically selected to be between 1 and 10, such as 1, 2, 3, 4, 5, 6, 7, 8, 9 and 10 amino acids bearing an aryl amine.

The incorporation of amino acids bearing aryl amines onto peptide-based hydrophobic polymers, both when the hydrophobic polymer is produced alone or on-resin as an amphiphilic block copolymer, should be of a high enough number to improve solubility of the peptide sequence in aqueous miscible organic solvents and should be sufficient to promote particle assembly when used as the dominant monomer units of hydrophobic polymers.

Unexpectedly, the inventors of the present disclosure found that between 1 and 10, such as 1, 2, 3, 4, 5, 6, 7, 8, 9 and 10 amino acids bearing aryl amine groups were sufficient to improve manufacturability and solubility of peptide-based hydrophobic polymers, but that between 3 and 30, such as 3, 4, 5, 6, 7, 8, 9, 10, 11, 12, 13, 14, 15, 16, 17, 18, 19, 20, 21, 22, 23, 24, 25, 26, 27, 28, 29 or 30, were preferable when such amino acids were the dominant and/or majority monomer unit in the hydrophobic polymer. Therefore, in preferred embodiments, the number of amino acids bearing aryl amines incorporated into peptide-based hydrophobic polymers produced on-resin is typically selected to be between 3 and 30. Of note, though, peptide-based hydrophobic polymers comprising more than 30 amino acids, typically more than 50 amino acids, are preferably prepared by convergent assembly of two or more peptides produced by solid-phase peptide synthesis or are prepared by an alternative route, such as by ring opening polymerization.

### EXAMPLES

### Example 1: Solid-Phase Synthesis of Poly(Amino Acid)-Based Hydrophobic Polymers or Oligomers (H) Used in the Assembly of Amphiphilic Block Copolymers

Hydrophobic polymers or oligomers based on hydrophobic poly(amino acids) produced by solid phase peptide synthesis (SPPS) provide the advantage over hydrophobic polymers produced by radical polymerization that the resulting material obtained is chemically defined, i.e. a single product with an exact composition can be obtained.

However, a potential limitation of producing hydrophobic poly(amino acids) by SPPS is that highly hydrophobic peptides may not be soluble in the solvents commonly used for peptide coupling (e.g., DMF) and/or the hydrophobic peptides may not be suitable for purification using common HPLC mobile (e.g., acetonitrile and water) and stationary (e.g., C18) phases.

Therefore, we investigated the suitability of different hydrophobic poly(amino acids) based on amino acids having an aliphatic (Aliph), aromatic (Ar), heterocyclic aromatic (H—Ar) or aromatic amine (Ar-a) side chain for synthesis by SPPS and purification by HPLC (Table 1).

TABLE 1
hydrophobic poly(amino acids)

| Cmpd | | SEQ ID | | | Syn- | |
|---|---|---|---|---|---|---|
| # | Amino acid sequence | NO: | Length | Series | thesized | Purity |
| 1 | LLLLL | 31 | 5 | Aliph | Y | Crude |
| 2 | LLLLLLLLLL | 32 | 10 | Aliph | N | — |
| 3 | FFFFF | 33 | 5 | Ar | Y | 98% |
| 4 | FFFFFFFFFF | 34 | 10 | Ar | N | — |
| 5 | HHHHH | 35 | 5 | H-Ar | Y | Crude |
| 6 | HHHHHHHHHH | 36 | 10 | H-Ar | Y | Crude |
| 7 | WWWWW | 37 | 5 | H-Ar | Y | >95% |
| 8 | WWWWWWWWWW | 38 | 10 | H-Ar | Y | Crude |
| 9 | F′F′F′F′F | 39 | 5 | Ar-a | Y | >95% |
| 10 | F′F′F′F′F′F′F′F′F′F | 40 | 10 | Ar-a | Y | >95% |
| 11 | F′F′F′F′F′F′F′F′F′F′F′F′F′F′F′F′F′F′F′F′ | 41 | 20 | Ar-a | Y | >90% |
| 12 | F′F′F′F′F′F′F′F′F′F′F′F′F′F′F′F′F′F′F′F′F′F | 42 | 30 | Ar-a | Y | >90% |
| 13 | FWFWFWFWFW | 43 | 10 | H-Ar | N | — |
| 14 | WF′WF′WF′WF′WF′WF′WF′WF′WF′WF′ | 44 | 20 | H/Ar-a | Y | >90% |

L = leucine; F = phenylalanine; H = histidine; W = tryptophan; F′ = para-aminophenylalanine Aliph = aliphatic-based poly(amino acid); Ar = aromatic poly(amino acid); H-Ar = heterocyclic aromatic poly(amino acid); Ar-a = aromatic amine poly(amino acid); Y indicates successful synthesis upon first attempt; N indicates that the synthesis or purification of the specific amino acid sequence was not successful on the first attempt. Purity is the % AUC of the product determined by HPLC. Crude purity indicates that HPLC purification was not successful but crude material comprising the designated poly(amino acid)-based hydrophobic polymer or oligomer (H) was obtained.

Our results show that hydrophobic poly(amino acids) comprising amino acids with aromatic amine (Ar-a) side chains were the most readily accessible by SPPS, followed by poly(amino acids) comprising aromatic heterocycle (H—Ar), aromatic ring (Ar) and aliphatic side chains (Aliph). Notably, poly(amino acids) comprising aliphatic side chains posed the greatest challenges to production by SPPS, while use of amino acids comprising aromatic side chains were generally more accessible, with poly(amino acids) comprising heterocyclic aromatic and aromatic amine groups being the most readily accessed.

These results indicate that hydrophobic polymers or oligomers (H) comprising poly(amino acids) should include 1 or more amino acids with aromatic amines to improve synthesis and solubility during both purification by HPLC as well as for improved solubility in water miscible solvents.

### Example 2—Synthesis of Hydrophobic Polymers or Oligomers (H) with DBCO Linker Groups

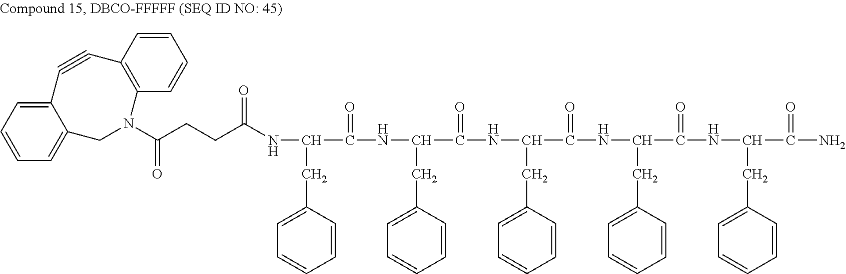 [SMILES: NC(=O)C(Cc1ccccc1)NC(=O)C(Cc1ccccc1)NC(=O)C(Cc1ccccc1)NC(=O)C(Cc1ccccc1)NC(=O)C(Cc1ccccc1)NC(=O)CCC(=O)N1Cc2ccccc2C#Cc2ccccc21]

Compound 15, referred to as DBCO-F₅, F₅ or DBCO-(Phe)s was synthesized by reacting 50.0 mg (0.066 mmol, 1 eq) of the precursor NH₂-(Phe)₅-NH₂ that was prepared by solid phase peptide synthesis with 29.4 mg of DBCO-NHS (0.073 mmol, 1.1 eq) and 7.4 mg of triethylamine (0.073 mmol, 1.1 eq) in 1.0 mL of DMSO. Compound 15 was purified on a preparatory HPLC system using a gradient of 30-95% acetonitrile/H₂O (0.05% TFA) over 12 minutes on an Agilent Prep-C18 column, 50×100 mm, 5 μm. The product eluted at ˜10 minutes and the resulting fractions were collected, frozen and then lyophilized to obtain a spectroscopically pure (>95% AUC at 254 nm) white powder. MS (ESI) calculated for C₆₄H₆₁N₇O₇ m/z 1039.46, found 1040.6 (M+H)⁺.

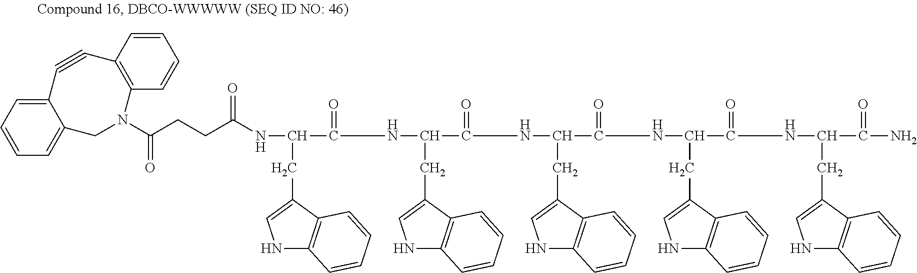 [SMILES: NC(=O)C(Cc1c[nH]c2ccccc12)NC(=O)C(Cc1c[nH]c2ccccc12)NC(=O)C(Cc1c[nH]c2ccccc12)NC(=O)C(Cc1c[nH]c2ccccc12)NC(=O)C(Cc1c[nH]c2ccccc12)NC(=O)CCC(=O)N1Cc2ccccc2C#Cc2ccccc21]

Compound 16, referred to as DBCO-W₅, W₅ or DBCO-(Trp)₅ was synthesized by reacting 137.6 mg (0.15 mmol, 1 eq) of the precursor NH₂-(Trp)₅-NH₂ that was prepared by solid phase peptide synthesis with 146.1 mg of DBCO-NHS (0.057 mmol, 2.5 eq) and 14.7 mg of triethylamine (0.15 mmol, 1.1 eq) in 3.0 mL of DMSO. Compound 16 was purified on a preparatory HPLC system using a gradient of 52-72% acetonitrile/H₂O (0.05% TFA) over 12 minutes on an Agilent Prep-C18 column, 50×100 mm, 5 μm. The product eluted at ˜10 minutes and the resulting fractions were collected, frozen and then lyophilized to obtain 75.1 mg (42% yield) of a spectroscopically pure (>95% AUC at 254 nm) white powder. MS (ESI) calculated for C₇₄H₆₆N₁₂O₇ m/z 1234.52, found 1235.6 (M+H)⁺.

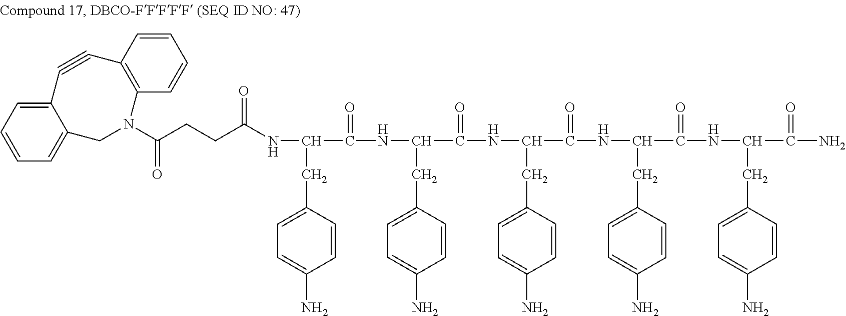 [SMILES: NC(=O)C(Cc1ccc(N)cc1)NC(=O)C(Cc1ccc(N)cc1)NC(=O)C(Cc1ccc(N)cc1)NC(=O)C(Cc1ccc(N)cc1)NC(=O)C(Cc1ccc(N)cc1)NC(=O)CCC(=O)N1Cc2ccccc2C#Cc2ccccc21]

Compound 17, referred to as DBCO-F′₅ or F′₅ was synthesized by reacting 49.8 mg (0.06 mmol, 1 eq) of the precursor NH₂—(F′)₅—NH₂, which was prepared by solid phase peptide synthesis, with 24.5 mg of DBCO-TT (0.057 mmol, 1.0 eq) and 30.3 mg of NaHCO₃ (0.36 mmol, 6.0 eq) in 1.0 mL of DMF. The reaction was run overnight at room temperature and HPLC indicated that the reaction was complete by 24 hours. Compound 17 was purified on a preparatory HPLC system using a gradient of 10-30% acetonitrile/H₂O (0.05% TFA) over 10 minutes on an Agilent Prep-C18 column, 30×100 mm, 5 μm. The product eluted at ˜3.4 minutes and the resulting fractions were collected, frozen and then lyophilized to obtain 25.8 mg (38.4% yield) of a spectroscopically pure (>95% AUC at 254 nm) white powder. MS (ESI) calculated for C₆₄H₆₆N₁₂O₇ m/z 1114.52, found 1116.1 (M+H)⁺.

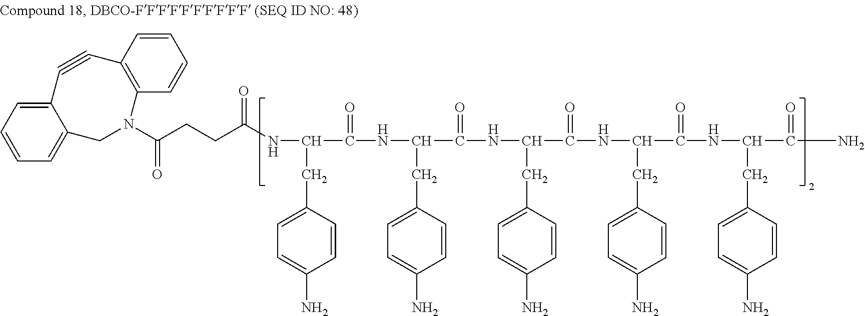

Compound 18, referred to as DBCO-F′₁₀ or F′₁₀ was synthesized by reacting 30 mg (0.0183 mmol, 1 eq) of the precursor NH₂—(F′)₁₀—NH₂, which was prepared by solid phase peptide synthesis, with 7.4 mg of DBCO-TT (0.018 mmol, 1.0 eq) and 16.9 mg of NaHCO₃ (0.20 mmol, 11 eq) in 1.0 mL of DMF. The reaction was run overnight at room temperature and HPLC indicated that the reaction was complete by 24 hours. Compound 18 was purified on a preparatory HPLC system using a gradient of 10-30% acetonitrile/H₂O (0.05% TFA) over 10 minutes on an Agilent Prep-C18 column, 30×100 mm, 5 μm. The product eluted at ˜6.3 minutes and the resulting fractions were collected, frozen and then lyophilized to obtain 14 mg (39.5% yield) of a spectroscopically pure (>95% AUC at 254 nm) white powder. MS (ESI) calculated for C₁₀₉H₁₁₆N₂₂O₁₂ m/z 1924.91, found 963.9 (M+2H)⁺.

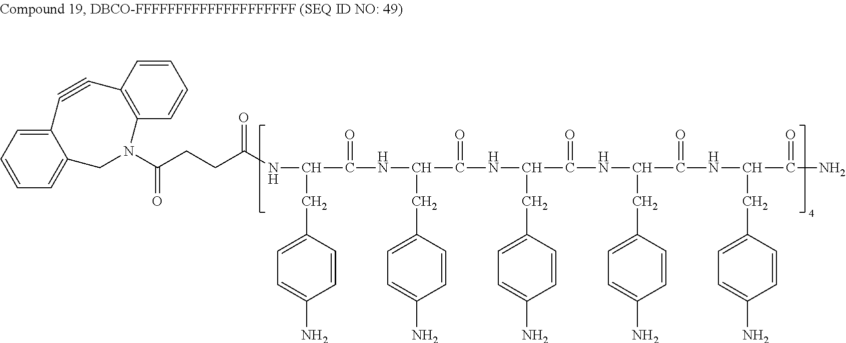

Compound 19, referred to as DBCO-F′₂₀ or F′₂₀ was synthesized by reacting 30 mg (0.009 mmol, 1 eq) of the precursor NH₂—(F′)₂₀—NH₂, which was prepared by solid phase peptide synthesis, with 3.7 mg of DBCO-TT (0.009 mmol, 1.0 eq) and 16.2 mg of NaHCO₃ (0.20 mmol, 21 eq) in 1.0 mL of DMF. The reaction was run overnight at room temperature and HPLC indicated that the reaction was complete by 24 hours. Compound 19 was purified on a preparatory HPLC system using a gradient of 10-30% acetonitrile/H₂O (0.05% TFA) over 10 minutes on an Agilent Prep-C18 column, 30×100 mm, 5 μm. The product eluted at ˜6.3 minutes and the resulting fractions were collected, frozen and then lyophilized to obtain 10.6 mg (32.4% yield) of a spectroscopically pure (>95% AUC at 254 nm) white powder. MS (ESI) calculated for C₁₉₉H₂₁₆N₄₂O₂₂ m/z 3545.71 found 1183.6 (M+3H)+ and 887 (M+4H)⁺.

### Example 3—Synthesis of Amphiphilic Block Copolymers by Reacting Azide-Functionalized S-[B] with DBCO-H to Produce S-[B]-H

As a facile process for producing amphiphilic block copolymers of formula S-[B]-H, azide functionalized S-[B] were reacted with DBCO functionalized hydrophobic polymers or oligomers (H). As an example, 0.5 mg of compound 15 in DMSO at 20 mg/mL was reacted with 1.0 mole equivalents of the peptide, KKSLVRX (SEQ ID NO:50), where X=azidolysine, which resulted in the complete conversion of starting material to compound 20:

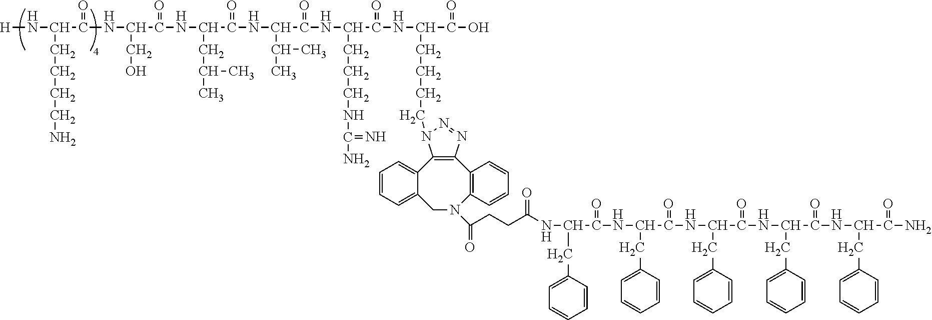

Similar reaction conditions were used to produce compounds 20-32 summarized in Table 2. Notable findings were that (i) amphiphilic block copolymers comprising aromatic amines typically resulted in stable micelles (˜20 nm size) without resulting in visible aggregates (i.e. turbidity >0.05 at 490 nm) or supramolecular associates (i.e. particle sizes >30 nm) and that (ii) net charge of the amphiphile greater than +4 or less than −4 were critical as PEG-modified hydrophobic polymers or oligomers (H) resulted in aggregates, which was not observed for the hydrophobic polymers or oligomers bearing a surface stabilizing group (S) comprised of a charged molecule (C).

TABLE 2
Size and stability of particles formed by amphiphilic block copolymers of formula S-
[B]-H

| | | SEQ | | | |
|---|---|---|---|---|---|
| | Sequence* | ID | | Aggregation | Particle size |
| # | S-B-(linker)-H | NO: | MW | (OD > 0.05) | (Diameter, nm) |
| 20 | KKK-SLVRX-(N3-DBCO)-FFFFF | 51 | 2178.9 | AGGREGATE | — |
| 21 | EEEEE-(N3-DBCO)-FFFFF | 52 | 1828.19 | AGGREGATE | — |
| 22 | KKK-SLVRX-(N3-DBCO)-WWWWW | 53 | 2373.96 | SOLUBLE | 64.73 |
| 23 | EEEEE-(N3-DBCO)-WWWWW | 54 | 2023.25 | SOLUBLE | 54.09 |
| 24 | KKK-SLVRX-(N3-DBCO)-F′F′F′F′F | 55 | 2253.96 | SOLUBLE | 12.40 |
| 25 | EEEEE-(N3-DBCO)-F′F′F′F′F′ | 56 | 1903.25 | SOLUBLE | 14.79 |
| 26 | KKK-SLVRX-(N3-DBCO)-F′F′F′F′F′F′F′F′F′F′ | 57 | 3064.35 | SOLUBLE | 15.71 |
| 27 | EEEEE-(N3-DBCO)-F′F′F′F′F′F′F′F′F′F′ | 58 | 2713.64 | SOLUBLE | 17.46 |
| 28 | OH-PEG24-(N3-DBCO)-F′F′F′F′F′F′F′F′F′F′ | 59 | 3025.2 | AGGREGATE | — |
| 29 | DDDDDDD-PEG24-(N3-DBCO)- | 60 | 4002.03 | SOLUBLE | 16.04 |
| | F′F′F′F′F′F′F′F′F′F′ | | | | |
| 30 | KKKKKKKKKKX-(N3-DBCO)- | 61 | 1452.93 | SOLUBLE | 8.41 |
| | F′F′F′F′F′F′F′F′F′F′ | | | | |
| 31 | KKK-SLVRX-(N3-DBCO)- | 62 | 4685.15 | SOLUBLE | 18.01 |
| | F′F′F′F′F′F′F′F′F′F′F′F′F′F′F′F′F′F′F′F′ | | | | |
| 32 | EEEEE-(N3-DBCO)- | 63 | 4334.44 | AGGREGATE | — |
| | F′F′F′F′F′F′F′F′F′F′F′F′F′F′F′F′F′F′F′F′ | | | | |

*Single letter code is used for the amine acid sequences listed in table 2; K = Lysine, S = Serine, L = leucine, V = valine, R = arginine, x = azidolysine, F = phenylalanine, W = tryptophan and F′ para-aminophenylalanine. (N3-DBCO) is the triazole linker that results from the reaction of an azide with DBCO. PEG24 refers to an ethylene oxide linker with 24 repeats.

An additional notable finding was that stable micelles could be formed by amphiphilic block copolymers comprising as few as 5 aromatic groups. Additional studies (data not shown) revealed, unexpectedly, that amphiphilic block copolymers with hydrophobic polymers or oligomers based on short hydrophobic oligomers, with as few as 3 amino acids with aromatic side chains, was sufficient to induce stable nanoparticle assembly; however, hydrophobic polymers or oligomers (H) with between 5 to 30 amino acids with aromatic side chains were identified to be preferable as amphiphilic block copolymers with 5 or more aromatic groups displayed lower critical micellar concentration, indicating improved stability of the micelles formed with longer length of poly(amino acid) chains.

### Example 4—Conjugation of Drug Molecules (D) to Hydrophobic Polymers or Oligomers (H)

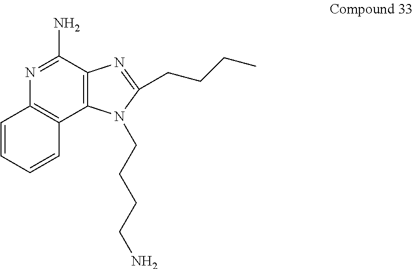 [SMILES: CCCCc1nc2c(N)nc3ccccc3c2n1CCCCN]

Compound 33, sometimes referred to as “2B,” 1-(4-aminobutyl)-2-butyl-1H-imidazo[4,5-c]quinolin-4-amine, referred to as 2B, was synthesized starting from 3-nitro-2,4-dichloroquinoline, 33-b, which was prepared as previously described (Lynn G M, et al., Nat Biotechnol 33(11):1201-1210, 2015). To 21 g of 33-b (87.8 mmol, 1 eq) in 210 mL of triethylamine (TEA) (10% w/w) was added 16.34 g (87.8 mmol, 1 eq) of N-boc-1,4-butanediamine while stirring vigorously. The reaction mixture was heated to 70° C. and monitored by HPLC, which confirmed that the reaction was complete after 2 hours. The triethylamine was removed under vacuum and the resulting oil was dissolved in 200 mL of dichloromethane and then washed with 3×100 mL DI H₂O. The organic layer was dried with Na₂SO₄ and then removed under vacuum and the resulting oil was triturated with 1:1 (v:v) hexane and diethyl ether to yield 30.7 g of yellow crystals of intermediate 33-c. MS (APCI) calculated for C₁₁H₂₃ClN₄O₄, m/z 394.1 found, 394.9.

33-d. 30.7 g (76.4 mmol) of intermediate 33-c was dissolved in 300 mL of ethyl acetate in a Parr Reactor vessel that was bubbled with argon, followed by the addition of 3 g of 10% platinum on carbon. The reaction vessel was kept under argon and then evacuated and pressurized with H₂(g) several times before pressurizing to 55 PSI H₂(g) while shaking vigorously. The H₂(g) was continually added until the pressure stabilized at 55 PSI, at which point the reaction was determined to be complete. The reaction mixture from the Parr Reactor was then filtered through celite end evaporated to dryness to obtain a yellow oil that was triturated with 1:1 hexanes/ether to yield white crystals that were collected by filtration to obtain 27.4 g of spectroscopically pure white crystals of 33-d. MS (APCI) calculated for C₁₈H₂₅ClN₄O₂, m/z 364.2, found 365.2.

33-e. To 10 g (27.4 mmol, 1 eq) of 33-d in 50 mL of THF was added 7.7 mL of triethylamine (54.8 mmol, 2 eq) followed by the drop wise addition of 3.6 g of valeroyl chloride (30.1 mmol, 1.1 eq) in 30 mL of THF while stirring vigorously while the reaction mixture was on ice. After 90 minutes, the ice bath was removed and the THF was removed under vacuum, resulting in a yellow oil that was dissolved in 100 mL of dichloromethane (DCM) that was washed with 3×50 mL of pH 5.5 100 mM acetate buffer. The DCM was removed under vacuum in an oil that was triturated with ethyl acetate to obtain 10.4 g of a white solid that was dissolved in methanol with 1 g of CaO (s), which was heated at 100° C. for 5 hours while stirring vigorously. The reaction mixture was filtered and dried to yield 10.2 g of an off-white solid, intermediate, 33-e. MS (ESI) calculated for C₂₃H₃₁ClN₄O₂, m/z 430.21, found 431.2.

33-f. To 10.2 g (23.7 mmol, 1 eq) of 1-e was added 30.4 g (284 mmol, 12 eq) of benzylamine liquid, which was heated to 110° C. while stirring vigorously. The reaction was complete after 10 hours and the reaction mixture was added to 200 mL ethyl acetate and washed 4×100 mL with 1 M HCl. The organic layer was dried with Na₂SO₄ and then removed under vacuum and the resulting oil was recrystallized from ethyl acetate to obtain 10.8 g of spectroscopically pure white crystals of intermediate, 33-f. MS (ESI) calculated for C₃₀H₃₉N₅O₂, m/z 501.31, found 502.3

Compound 33. 10.8 g (21.5 mmol) of 33-f was dissolved in 54 mL of concentrated (>98%) H₂SO₄ and the reaction mixture was stirred vigorously for 3 hours. After 3 hours, viscous red reaction mixture was slowly added to 500 mL of DI H₂O while stirring vigorously. The reaction mixture was stirred for 30 minutes and then filtered through Celite, followed by the addition of 10 M NaOH until the pH of the solution was ˜pH 10. The aqueous layer was then extracted with 6×200 mL of DCM and the resulting organic layer was dried with Na₂SO₄ and reduced under vacuum to yield a spectroscopically pure white solid. ¹H NMR (400 MHZ, DMSO-d6) δ 8.03 (d, J=8.1 HZ, 1H), 7.59 (d, J=8.1 Hz, 1H), 7.41 (t, J=7.41 Hz, 1H), 7.25 (t, J=7.4 Hz, 1H), 6.47 (s, 2H), 4.49 (t, J=7.4 Hz, 2H), 2.91 (t, J=7.78 Hz, 2H), 2.57 (t, J=6.64 Hz, 1H), 1.80 (m, 4H), 1.46 (sep, J=7.75 Hz, 4H), 0.96 (t, J=7.4 Hz, 3H). MS (ESI) calculated for C₁₈H₂₅N₅, m/z 311.21, found 312.3.

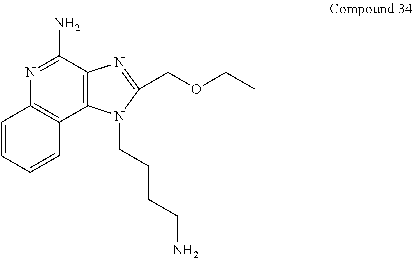 [SMILES: CCOCc1nc2c(N)nc3ccccc3c2n1CCCCN]

Compound 34 was prepared as previously described (Lynn G M, et al., Nat Biotechnol 33(11):1201-1210, 2015). ¹H NMR (400 MHz, DMSO-d6) δ 8.02 (dd, J=16.6, 8.2 Hz, 1H), 7.63-7.56 (m, 1H), 7.47-7.38 (m, 1H), 7.30-7.21 (m, 1H), 6.55 (s, 2H), 4.76 (s, 2H), 4.54 (q, J=6.3, 4.4 Hz, 2H), 3.54 (q, J=7.0 Hz, 2H), 2.58 (t, J=6.9 Hz, 2H), 1.93-1.81 (m, 2H), 1.52 (m, 2H), 1.15 (t, J=7.0 Hz, 3H). MS (APCI) calculated for C₁₇H₂₃N₅O m/z 313.2, found 314.2 (M+H)⁺.

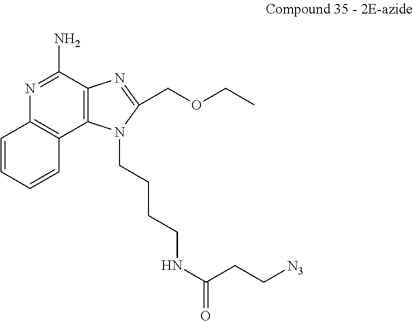 [SMILES: CCOCc1nc2c(N)nc3ccccc3c2n1CCCCNC(=O)CCN=[N+]=[N-]]

Compound 35 was prepared as previously described (Lynn G M, et al., Nat Biotechnol 33(11):1201-1210, 2015). MS (APCI) calculated for C₂₀H₂₆N₈O₂ m/z 410.2, found 411.2 (M+H)⁺.

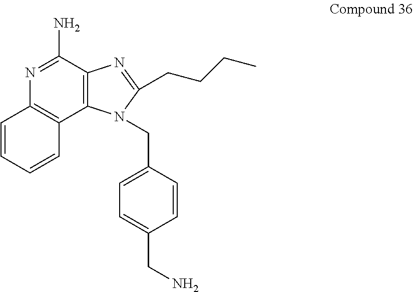 [SMILES: CCCCc1nc2c(N)nc3ccccc3c2n1Cc1ccc(CN)cc1]

Compound 36, 1-(4-(aminomethyl)benzyl)-2-butyl-1H-imidazo[4,5-c]quinolin-4-amine, referred to as 2BXy, was previously described (see: Lynn G M, et al., In vivo characterization of the physicochemical properties of polymer-linked TLR agonists that enhance vaccine immunogenicity. Nat Biotechnol 33(11):1201-1210, 2015, and Shukla N M, et al. Syntheses of fluorescent imidazoquinoline conjugates as probes of Toll-like receptor 7. Bioorg Med Chem Lett 20(22):6384-6386, 2010). 1H NMR (400 MHz, DMSO-d6) δ 7.77 (dd, J=8.4, 1.4 Hz, 1H), 7.55 (dd, J=8.4, 1.2 Hz, 1H), 7.35-7.28 (m, 1H), 7.25 (d, J=7.9 Hz, 2H), 7.06-6.98 (m, 1H), 6.94 (d, J=7.9 Hz, 2H), 6.50 (s, 2H), 5.81 (s, 2H), 3.64 (s, 2H), 2.92-2.84 (m, 2H), 2.15 (s, 2H), 1.71 (q, J=7.5 Hz, 2H), 1.36 (q, J=7.4 Hz, 2H), 0.85 (t, J=7.4 Hz, 3H). MS (APCI) calculated for C₂₂H₂₅N₅ m/z 359.2, found 360.3

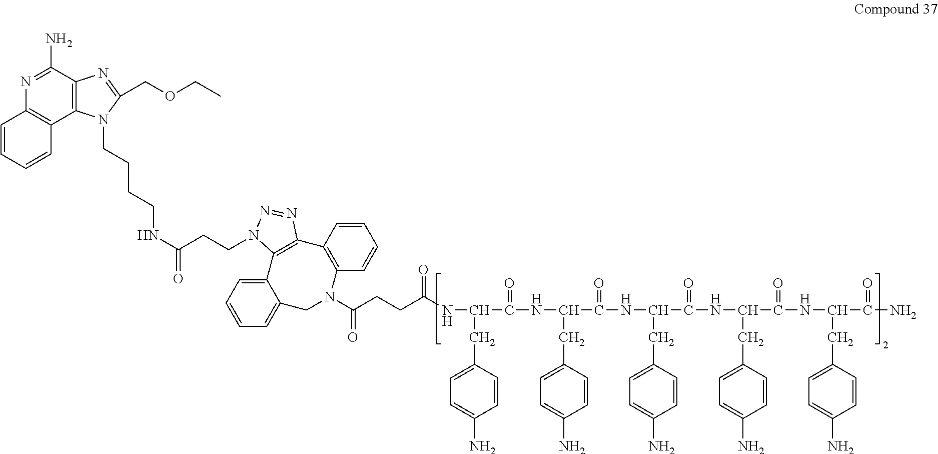

Compound 37 was produced by reacting 0.5 mg of compound 15 in DMSO at 20 mg/mL with 1.0 mole equivalents of compound 35, which resulted in the complete conversion of starting material to compound 37.

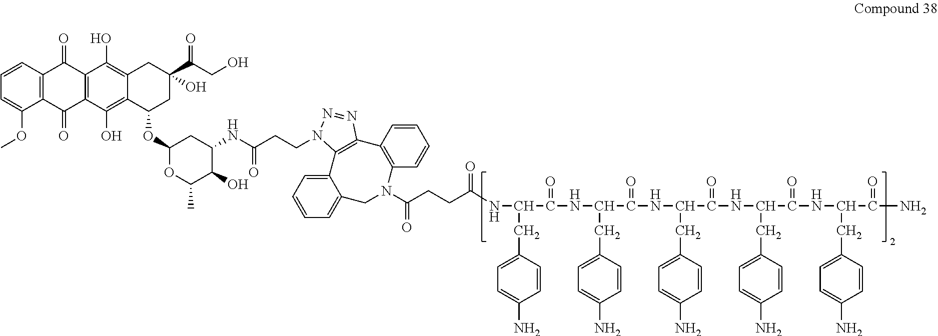

Compound 38 was produced by reacting 0.5 mg of compound 15 in DMSO at 20 mg/mL with 1.0 mole equivalents of azide functionalized doxorubicin, which resulted in the complete conversion of starting material to compound 38.

### Example 5—Synthesis of X1-H(D)

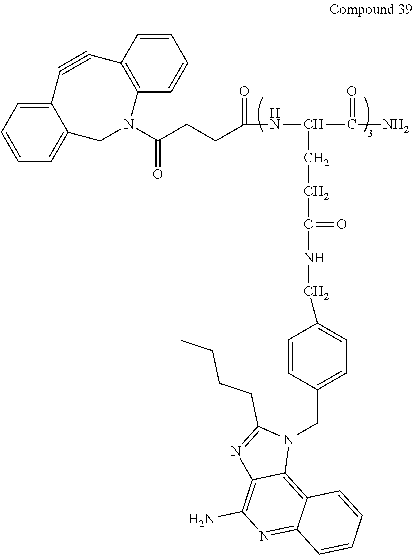

Compound 39, referred to as DBCO-2BXy₃, 2BXy₃ or DBCO-(Glu(2BXy)₃), was synthesized starting from an Fmoc-(Glu)₃-NH₂ precursor prepared by solid-phase peptide synthesis. 50 mg of Fmoc-(Glu)₃-NH₂ (0.08 mmol, 1 eq), 143 mg of Compound 36 (0.40 mmol, 5 eq), 84 mg of 2-chloro-4,6-dimethoxy-1,3,5-triazine (CDMT) (0.48 mmol, 6 eq) and 48.5 mg of 4-methylmorpholine (NMM) (0.48 mmol, 6 eq) were added to 3.25 mL of DMSO while stirring vigorously at room temperature under ambient air. The reaction progress was monitored by HPLC (AUC 254 nm). 1 additional equivalent of Compound 36 and 2 additional equivalents of both CDMT and NMM were added after 30 minutes. After 2 hours, the reaction was complete and the reaction mixture was added to 50 mL of a 1M HCl solution to precipitate the Fmoc protected intermediate, which was collected by centrifuging the solution at 3000 g at 4° C. for 10 minutes. The HCl solution was discarded and the Fmoc protected intermediate was collected as a solid white pellet. The white solid was re-suspended in 50 mL of a 1M HCl solution and spun at 3000 g at 4° C. for 5 minutes; the 1 M HCl solution was discarded and the product was collected as a solid pellet. This process was repeated and then the solid was collected and dried under vacuum to yield 156.1 mg of the Fmoc protected intermediate in quantitative yield. The Fmoc protected product was then added to 1.5 mL of a 20% piperidine in DMF solution for 30 minutes at room temperature to yield the deprotected product that was then precipitated from 50 mL of ether and centrifuged at 3000 g at 4° C. for 30 minutes. The product was collected as a solid pellet and then washed twice more with ether, followed by drying under vacuum to yield 126.4 mg of the intermediate. 60 mg of the resulting intermediate, NH₂-(Glu-2BXy)₃-NH₂, (0.042 mmol, 1 eq) was then reacted with 18.6 mg (0.046 mmol, 1.1 eq) of DBCO-NHS ester (Scottsdale, Ariz., USA) and 8.5 uL of triethylamine (0.084 mmol, 2 eq) in 1 mL of DMSO for 6 hours at room temperature. The resulting product, Compound 39, was purified on a preparatory HPLC system using a gradient of 30-70% acetonitrile/H₂O (0.05% TFA) over 12 minutes on an Agilent Prep-C18 column, 50×100 mm, 5 μm. The product eluted at 7.0 minutes and the resulting fractions were collected, frozen and then lyophilized to obtain 40.12 mg (55.7% yield) of a spectroscopically pure (>95% AUC at 254 nm) white powder. MS (ESI) calculated for C₁₀₀H₁₀₆N₀₂O₈ m/z 1714.85, found 858.9 (M/2)+

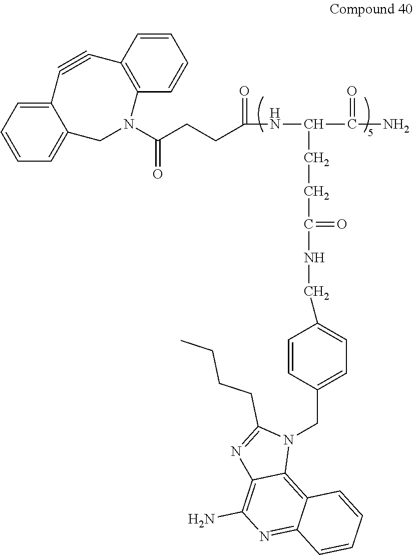

Compound 40, referred to as DBCO-2BXy₅, 2BXy₅ or DBCO-(Glu(2BXy)₅), was synthesized using the same procedure as described for Compound 39, except Fmoc-(Glu)₅-NH₂ was used as the starting material for conjugation of Compound 36. Compound 40 was purified on a preparatory HPLC system using a gradient of 38-48% acetonitrile/H₂O (0.05% TFA) over 12 minutes on an Agilent Prep-C18 column, 50×100 mm, 5 μm. The product eluted at 8.0 minutes and the resulting fractions were collected, frozen and then lyophilized to obtain 45.9 mg (63.4% yield) of a spectroscopically pure (>95% AUC at 254 nm) white powder. MS (ESI) calculated for C₁₅₄H₁₆₆N₃₂O₁₂ m/z 2655.34, found 886.6 (M/3)⁺.

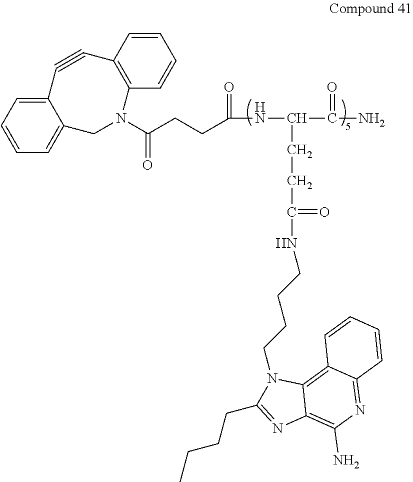

Compound 41, referred to as DBCO-2B₅, 2B₅ or DBCO-(Glu(2B)₅), was synthesized using the same procedure as described for Compound 39, except Fmoc-(Glu)₅-NH₂ (SEQ ID NO:64), was used as the starting material for conjugation of Compound 33. Compound 41 was purified on a preparatory HPLC system using a gradient of 33-45% acetonitrile/H₂O (0.05% TFA) over 12 minutes on an Agilent Prep-C18 column, 50×100 mm, 5 μm. The product eluted at ˜10.0 minutes and the resulting fractions were collected, frozen and then lyophilized to obtain 25.2 mg (62.6% yield) of a spectroscopically pure (>95% AUC at 254 nm) white powder. MS (ESI) calculated for C₁₃₄H₁₆₆N₃₂O₁₂ m/z 2415.34, found 1209.3 (M/2)⁺.

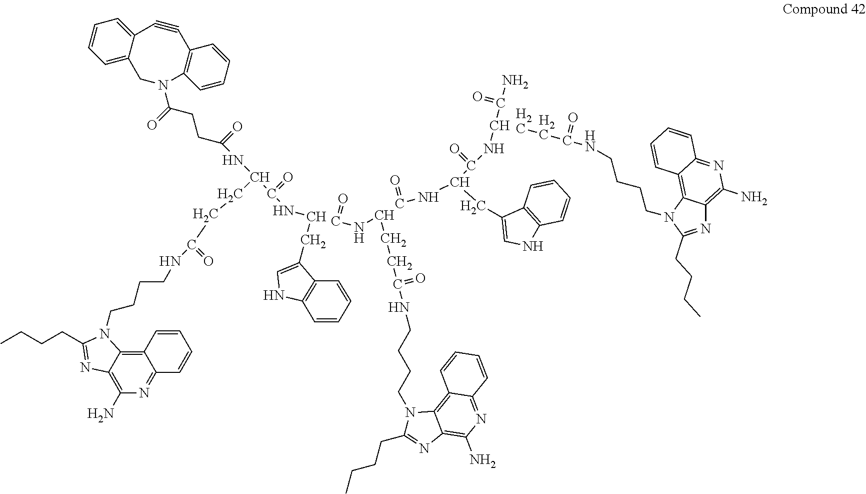

Compound 42, referred to as DBCO-2B₃W₂, 2B₃W₂ or DBCO-(Glu(2B)₃(Trp)₂), was synthesized using the same procedure as described for Compound 39, except Fmoc-Glu-Trp-Glu-Trp-Glu-NH₂ (SEQ ID NO:65), was used as the starting material for conjugation of Compound 33. Compound 42 was purified on a preparatory HPLC system using a gradient of 33-47% acetonitrile/H₂O (0.05% TFA) over 12 minutes on an Agilent Prep-C18 column, 50×100 mm, 5 m. The product eluted at ˜8 minutes and the resulting fractions were collected, frozen and then lyophilized to obtain 197 mg (50.6% yield) of a spectroscopically pure (>95% AUC at 254 nm) white powder. MS (ESI) calculated for C₁₁₀H₁₂₆N₂₄O₁₀ m/z 1943.01, found 973.0 (M/2)⁺.

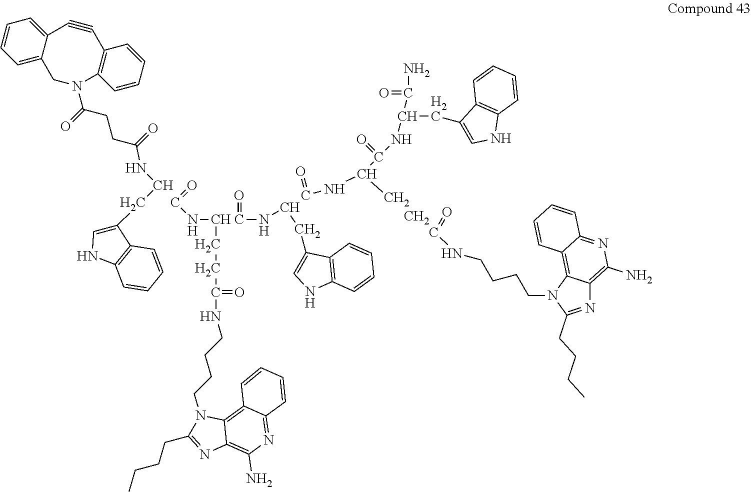

Compound 43, referred to as DBCO-2B₂W₃, 2B₂W₃ or DBCO-(Glu(2B)₂(Trp)₃), was synthesized using the same procedure as described for Compound 39, except Fmoc-Trp-Glu-Trp-Glu-Trp-NH₂ (SEQ ID NO:66), was used as the starting material for conjugation of Compound 33. Compound 43 was purified on a preparatory HPLC system using a gradient of 35-65% acetonitrile/H₂O (0.05% TFA) over 12 minutes on an Agilent Prep-C18 column, 50×100 mm, 5 μm. The product eluted at ˜9 minutes and the resulting fractions were collected, frozen and then lyophilized to obtain 11.6 mg (62.5% yield) of a spectroscopically pure (>95% AUC at 254 nm) white powder. MS (ESI) calculated for C₉₈H₁₀₆N₂₀O₉ m/z 1706.85, found 854.9 (M/2)⁺.

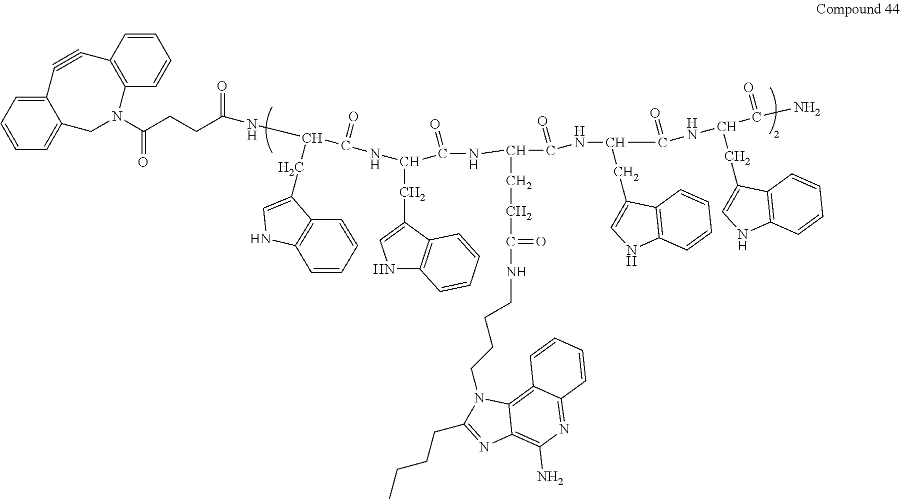

Compound 44, referred to as DBCO-2B₂W₈, 2B₂W₈ or DBCO-(Glu(2B)₂(Trp)s), was synthesized using the same procedure as described for Compound 39, except Fmoc-Trp-Trp-Glu-Trp-Trp-Trp-Trp-Glu-Trp-Trp-NH₂ (SEQ ID NO:67), was used as the starting material for conjugation of Compound 33. Compound 44 was purified on a preparatory HPLC system using a gradient of 35-85% acetonitrile/H₂O (0.05% TFA) over 12 minutes on an Agilent Prep-C18 column, 50×100 mm, 5 μm. The product eluted at ˜8.0 minutes and the resulting fractions were collected, frozen and then lyophilized to obtain 3.3 mg (16.3% yield) of a spectroscopically pure (>95% AUC at 254 nm) white powder. MS (ESI) calculated for C₁₅₃H₁₅₆N₃₀O₁₄ m/z 2637.24, found 1320.2 (M/2)⁺.

Compound 45

Compound 45 referred to as DBCO-2B₁W₄, 2B₁W₄ or DBCO-(Glu(2B)₁(Trp)₄), was

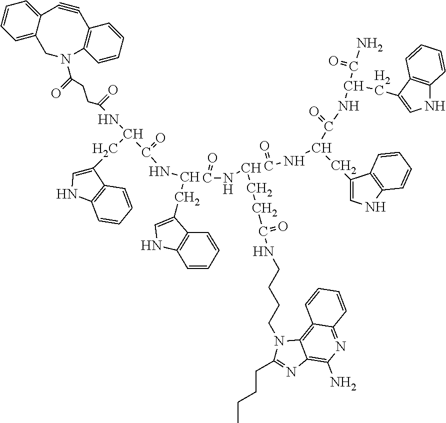

synthesized using the same procedure as described for Compound 39, except Fmoc-Trp-Trp-Glu-Trp-Trp-NH₂ (SEQ ID NO:68), was used as the starting material for conjugation of Compound 33. Compound 45 was purified on a preparatory HPLC system using a gradient of 50-55% acetonitrile/H₂O (0.05% TFA) over 12 minutes on an Agilent Prep-C18 column, 50×100 mm, 5 m. The product eluted at 8.9 minutes and the resulting fractions were collected, frozen and then lyophilized to obtain 9.7 mg (55.4% yield) of a spectroscopically pure (>95% AUC at 254 nm) white powder. MS (ESI) calculated for C₈₆H₈₆N₁₆O₈ m/z 1470.68, found 736.6 (M/2)⁺.

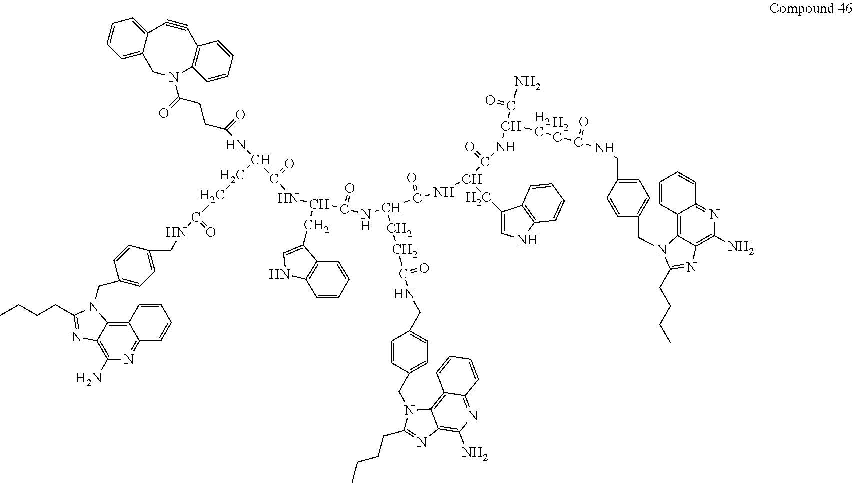 [SMILES: CCCCc1nc2c(N)nc3ccccc3c2n1Cc1ccc(CNC(=O)CCC(NC(=O)C(Cc2c[nH]c3ccccc23)NC(=O)C(CCC(=O)NCc2ccc(Cn3c(CCCC)nc4c(N)nc5ccccc5c43)cc2)NC(=O)C(Cc2c[nH]c3ccccc23)NC(=O)C(CCC(=O)NCc2ccc(Cn3c(CCCC)nc4c(N)nc5ccccc5c43)cc2)NC(=O)CCC(=O)N2Cc3ccccc3C#Cc3ccccc32)C(N)=O)cc1]

Compound 46, referred to as DBCO-2BXy₃W₂, 2BXy₃W₂ or DBCO-(Glu(2BXy)₃(Trp)₂), was prepared using Fmoc-Glu-Trp-Glu-Trp-Glu-NH₂ (SEQ ID NO:69) and Compound 33 as the starting materials. 500 mg of Fmoc-Glu-Trp-Glu-Trp-Glu-NH₂ (SEQ ID NO:69), (0.5 mmol, 1 eq), 595.6 mg of Thiazoline-2-Thiol (TT) (5 mmol, 10 eq), and 575.7 mg of 1-Ethyl-3-(3-dimethylaminopropyl)carbodiimide (EDC) (3 mmol, 6 eq) were suspended in 26 mL of DCM. 18.3 mg of 4-(Dimethylamino)pyridine (DMAP) (0.2 mmol, 0.3 eq) was added and the reaction mixture was stirred at room temperature. The reaction progress was monitored by analytical HPLC. After 4 hours, an additional four equivalents of TT and two equivalents of EDC were added. After stirring overnight, two equivalents of TT and a half equivalent of EDC were added. After 6 hours, the reaction was complete. The DCM was removed under vacuum and the solid was taken up in 6 mL of dry DMSO. 539.3 mg of Compound 33 (1.5 mmol, 3 eq) was added and the reaction mixture was stirred for 2 hours at room temperature. The conjugated intermediate was then precipitated from 300 mL of 1 M HCl and centrifuged at 3000 g at 4° C. for 10 minutes. The pellet was collected and washed once more with 1 M HCl and once with DI water. The final collected pellet was frozen and dried under vacuum. 809.06 mg of Fmoc-2BXy₃W₂—NH₂ (0.4 mmol, 1 eq)) was dissolved in 4 mL of 20% piperidine in DMF. The reaction mixture was stirred at room temperature for 1 hour. The deprotected intermediate was then precipitated from 100 mL of ether and centrifuged at 3000 g at 4° C. for 10 minutes. The product was collected as a solid pellet and then washed twice more with ether, followed by drying under vacuum to yield the intermediate. 729 mg NH₂-2BXy₃W₂—NH2 (0.4 mmol, 1 eq) was dissolved in 6 mL of dry DMSO. 488.8 mg of DBCO-NHS (1.2 mmol, 3 eq) was added and the reaction mixture was stirred at room temperature for 1 hour. The resulting product was purified on a preparatory HPLC system using a gradient of 36-46% acetonitrile/H2O (0.05% TFA) over 12 minutes on an Agilent Prep C-18 column, 50×100 mm, 5 μm. The resulting fractions were combined, frozen and lyophilized to give 239 mg (38.1% yield) of a spectroscopically pure of white powder. MS (ESI) Calculated for C₁₂₂H₁₂₆N₂₄O₁₀ m/z 2087.65 found 697 (m/3)⁺.

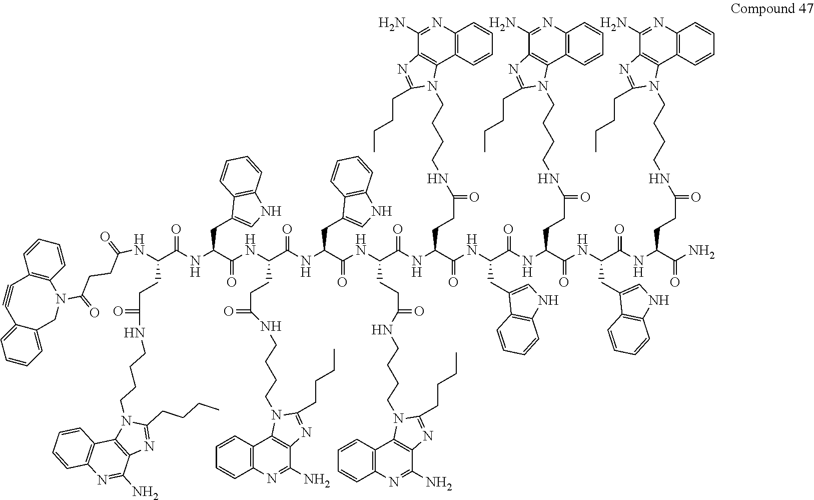 [SMILES: CCCCc1nc2c(N)nc3ccccc3c2n1CCCCNC(=O)CC[C@H](NC(=O)[C@H](Cc1c[nH]c2ccccc12)NC(=O)[C@H](CCC(=O)NCCCCn1c(CCCC)nc2c(N)nc3ccccc3c21)NC(=O)[C@H](Cc1c[nH]c2ccccc12)NC(=O)[C@H](CCC(=O)NCCCCn1c(CCCC)nc2c(N)nc3ccccc3c21)NC(=O)[C@H](CCC(=O)NCCCCn1c(CCCC)nc2c(N)nc3ccccc3c21)NC(=O)[C@H](Cc1c[nH]c2ccccc12)NC(=O)[C@H](CCC(=O)NCCCCn1c(CCCC)nc2c(N)nc3ccccc3c21)NC(=O)[C@H](Cc1c[nH]c2ccccc12)NC(=O)[C@H](CCC(=O)NCCCCn1c(CCCC)nc2c(N)nc3ccccc3c21)NC(=O)CCC(=O)N1Cc2ccccc2C#Cc2ccccc21)C(N)=O]

Compound 47, referred to as DBCO-2B₆W₄, 2B₆W₄ or DBCO-(Glu(2B)₆(Trp)₄), was synthesized using the same procedure as described for Compound 39, except Fmoc-(Glu-Trp-Glu-Trp-Glu)₂-NH₂ (SEQ ID NO:70), was used as the starting material for conjugation of Compound 33. Compound 47 was purified on a preparatory HPLC system using a gradient of 24-45% acetonitrile/H₂O (0.05% TFA) over 10 minutes on an Agilent Prep-C18 column, 30×100 mm, 5 m. The fractions were collected, frozen and then lyophilized to obtain a spectroscopically pure (>95% AUC at 254 nm) white powder. MS (ESI) calculated for C₂₀₁H₂₃₆N₄₆O₁₈ m/z 3582.4, found 717.7 (M/5)+

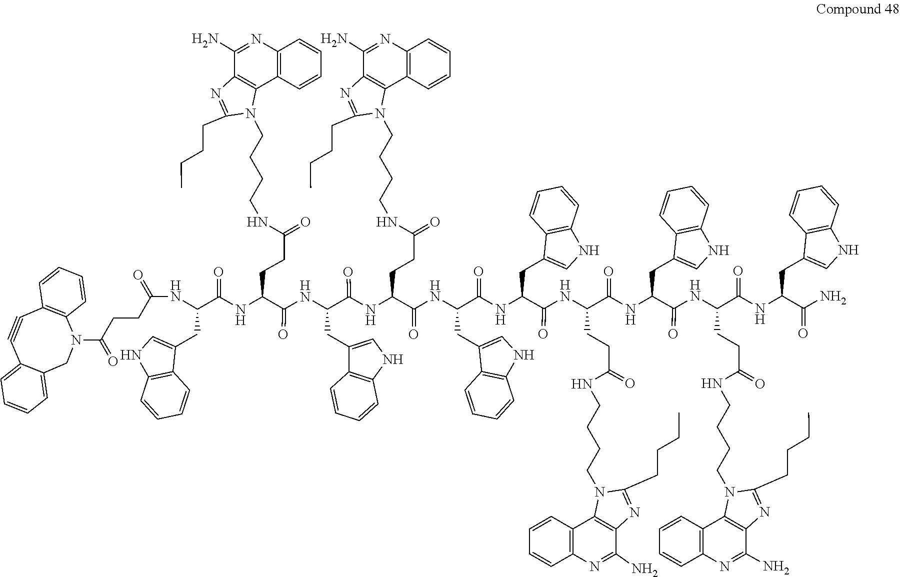 [SMILES: CCCCc1nc2c(N)nc3ccccc3c2n1CCCCNC(=O)CC[C@H](NC(=O)[C@H](Cc1c[nH]c2ccccc12)NC(=O)[C@H](CCC(=O)NCCCCn1c(CCCC)nc2c(N)nc3ccccc3c21)NC(=O)[C@H](Cc1c[nH]c2ccccc12)NC(=O)[C@H](Cc1c[nH]c2ccccc12)NC(=O)[C@H](CCC(=O)NCCCCn1c(CCCC)nc2c(N)nc3ccccc3c21)NC(=O)[C@H](Cc1c[nH]c2ccccc12)NC(=O)[C@H](CCC(=O)NCCCCn1c(CCCC)nc2c(N)nc3ccccc3c21)NC(=O)[C@H](Cc1c[nH]c2ccccc12)NC(=O)CCC(=O)N1Cc2ccccc2C#Cc2ccccc21)C(=O)N[C@@H](Cc1c[nH]c2ccccc12)C(N)=O]

Compound 48, referred to as DBCO-2B₄W₆, 2B₄W₆ or DBCO-(Glu(2B)₄(Trp)₆), was synthesized using the same procedure as described for Compound 39, except Fmoc-(Trp-Glu-Trp-Glu-Trp)₂-NH₂ (SEQ ID NO:71), was used as the starting material for conjugation of Compound 33. Compound 48 was purified on a preparatory HPLC system using a gradient of 24-45% acetonitrile/H₂O (0.05% TFA) over 10 minutes on an Agilent Prep-C18 column, 30×100 mm, 5 m. The fractions were collected, frozen and then lyophilized to obtain a spectroscopically pure (>95% AUC at 254 nm) white powder. MS (ESI) calculated for C₁₇₇H₁₉₆N₃₈O₁₆ m/z 3111.7, found 777.5 (M/4)⁺.

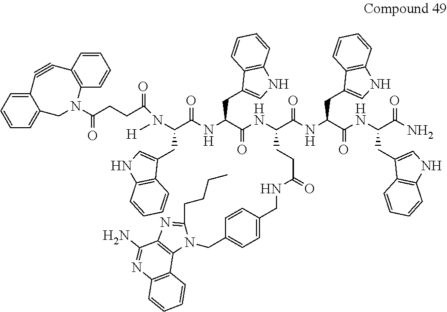 [SMILES: CCCCc1nc2c(N)nc3ccccc3c2n1Cc1ccc(CNC(=O)CC[C@H](NC(=O)[C@H](Cc2c[nH]c3ccccc23)NC(=O)[C@H](Cc2c[nH]c3ccccc23)NC(=O)CCC(=O)N2Cc3ccccc3C#Cc3ccccc32)C(=O)N[C@@H](Cc2c[nH]c3ccccc23)C(=O)N[C@@H](Cc2c[nH]c3ccccc23)C(N)=O)cc1]

Compound 49, referred to as DBCO-2BXy₁W₄, 2BXy₁W₄ or DBCO-(Glu(2BXy)₁(Trp)₄), was prepared using the same procedure as described for Compound 39, except Fmoc-Trp-Trp-Glu-Trp-Trp-NH₂ was used as the starting material. Compound 49 was purified on a preparatory HPLC system using a gradient of 40-70% acetonitrile/H₂O (0.05% TFA) over 16 minutes on an Agilent Prep-C18 column, 30×100 mm, 5 μm. The resulting fractions were collected, frozen and then lyophilized to obtain 3.4 mg (73.3% yield) of a spectroscopically pure (>95% AUC at 254 nm) white powder. MS (ESI) calculated for C₉₀H₈₅N₁₅O₉ m/z 1519.67, found 760.5 (M/2)⁺.

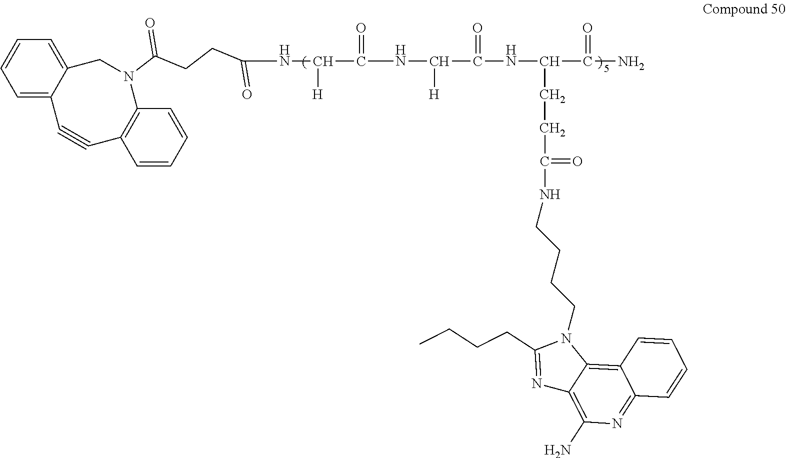

Compound 50, referred to as DBCO-(GG2B)₅, 2B₅G₁₀ or DBCO-(Glu(2B)₅(Gly)₁₀), was synthesized using the same procedure described for Compound 39, except Fmoc-(Gly-Gly-Glu)₅-NH₂ and Compound 33 were used as the starting materials. Compound 50 was purified on a preparatory HPLC system using a gradient of 22-42% acetonitrile/H₂O (0.05% TFA) over 12 minutes on an Agilent Prep-C18 column, 30×100 mm, 5 μm. The product eluted at 7 minutes and the resulting fractions were collected, frozen and then lyophilized to obtain 22.8 mg (36.2% yield) of a spectroscopically pure (>95% AUC at 254 nm) white powder. MS (ESI) calculated C₁₅₄H₁₉₆N₄₂O₂₂ for m/z 2985.51, found 598.5 (M/5)⁺.

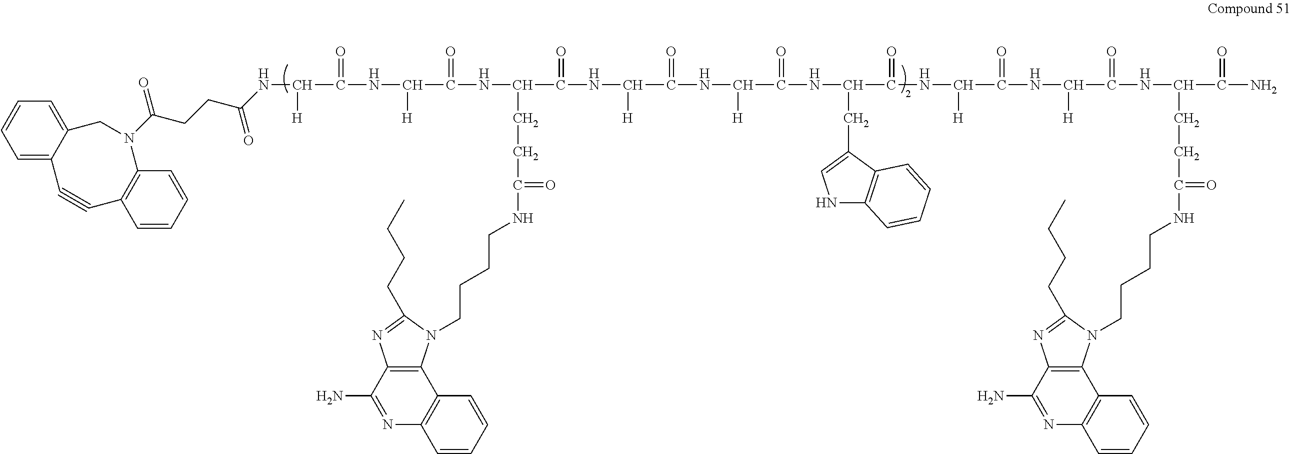

Compound 51, referred to as DBCO-(GG2BGGW)₂GG2B, 2B₃W₂G₁₀ or DBCO-(Glu(2B)₃(Trp)₂(Gly)₁₀), was synthesized from Fmoc-(Gly-Gly-Glu-Gly-Gly-Trp)₂-Gly₂-Glu-NH₂ (SEQ ID NO:76), precursor prepared by solid-phase peptide synthesis and Compound 33. 235.4 mg of Fmoc-(Gly-Gly-Glu-Gly-Gly-Trp)₂-Gly₂-Glu-NH₂ (SEQ ID NO:76), (0.15 mmol, 1 eq) was dissolved in 2 mL of 20% Piperidine in DMF. After 30 minutes the reaction was complete and the product was precipitated from 100 mL of ether and centrifuged at 3000 g at 4° C. for 10 minutes. The product was collected as a solid pellet and then washed twice more with ether, followed by drying under vacuum to yield ˜200 mg of the deprotected intermediate. 200 mg (0.15 mmol, 1 eq) of NH2-(Gly-Gly-Glu-Gly-Gly-Trp)₂-Gly₂-Glu-NH₂ (SEQ ID NO:77), was dissolved in 2 mL of dry DMSO and 89.73 mg of DBCO-NHS (0.22 mmol, 1.5 eq) was added followed by TEA (0.22 mmol, 1.5 eq). The reaction mixture was stirred at room temperature for 1 hour. The resulting DBCO intermediate was purified on a preparatory HPLC system using a gradient of 30-50% acetonitrile/H2O (0.05% TFA) over 12 minutes on an Agilent Prep C-18 column, 50×100 mm, 5 μm. The resulting fractions were combined, frozen and lyophilized to give the intermediate. 25 mg of DBCO-(Gly-Gly-Glu-Gly-Gly-Trp)₂-Gly₂-Glu-NH₂ (SEQ ID NO:78), (0.015 mmol, 1 eq) and 17.11 mg of Compound 33 (0.055 mmol, 3.6 eq) were dissolved in 1.2 mL of dry DMSO. TEA (0.183 mmol, 12 eq) was added and the reaction mixture was stirred at room temperature for 5 minutes. 19.17 mg of HATU (0.05 mmol, 3.3 eq) was added and the reaction mixture was stirred at room temperature. The progress of the reaction was monitored by LC-MS. 1.2 additional equivalents of Compound 33 and 1.1 equivalents HATU were added after 1 hour. After 2 hours, the reaction was complete. The resulting product was purified on a preparatory HPLC system using a gradient of 30-60% acetonitrile/H2O (0.05% TFA) over 12 minutes on an Agilent Prep C-18 column, 30×100 mm, 5 μm. The resulting fractions were combined, frozen and lyophilized to give a spectroscopically pure white powder. MS (ESI) Calculated for C₁₃₀H₁₅₆N₃₄O₂₀ m/z 2515.96 found 839 (m/3)⁺.

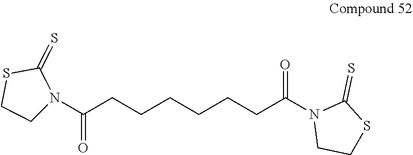 [SMILES: O=C(CCCCCCC(=O)N1CCSC1=S)N1CCSC1=S]

Compound 52, referred to as Bis(TT), was synthesized using Suberic acid and 2-thiazoline-2-thiol (TT) as starting materials. Briefly, 500 mg of Suberic acid (2.87 mmol, 1 eq), 752.7 mg of TT (6.31 mmol, 2.2 eq) and 1.431 g of EDC (7.46 mmol, 2.6 eq) were dissolved in 17.5 mL of dry DMSO. 70.15 mg of DMAP (0.57 mmol, 0.2 eq) was added and the reaction mixture was stirred at room temperature for 1 hour. The reaction mixture was diluted with DCM and washed twice with 1 M HCl and once with DI water. The organic fractions were dried with sodium sulfate and evaporated under reduced pressure to provide a yellow solid in quantitative yield.

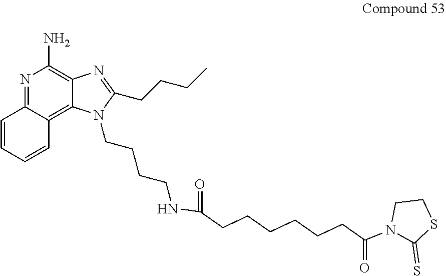 [SMILES: CCCCc1nc2c(N)nc3ccccc3c2n1CCCCNC(=O)CCCCCCC(=O)N1CCSC1=S]

Compound 53, referred to as 2B-TT, was synthesized using Compound 52 and Compound 33 as starting materials. Briefly, 50 mg (0.16 mmol, 1 eq) of Compound 33 was dissolved in 0.6 mL of methanol and added dropwise to a vigorously stirring solution of 301.1 mg of Compound 53 (0.8 mmol, 5 eq) in 1.93 mL of DCM. After 30 minutes, the reaction mixture was injected directly onto a column and purified by flash chromatography using a 2-step gradient: 5% methanol in DCM over 5 column volumes (CVs), followed by a 5-50% methanol in DCM gradient over 20 CVs. The fractions were combined and the solvent was removed under vacuum. MS (ESI) calculated for C₂₉H₄₀N₆O₂S₂ m/z 568.27 found 569.3 (m+H)⁺.

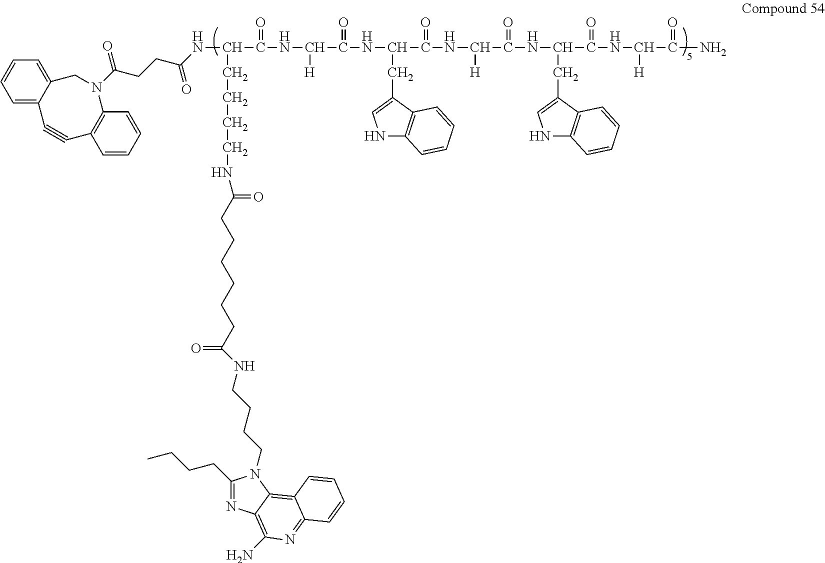

Compound 54 referred to as DBCO-(2BGWGWG)₅, 2B₅W₁₀G₁₅ or DBCO-(Glu(2B)₅(Trp)₁₀(Gly)₁₅), was synthesized from an Fmoc-(Lys-Gly-Trp-Gly-Trp-Gly)₅-NH₂ (SEQ ID NO:80), peptide precursor that was prepared by solid-phase peptide synthesis and Compound 53. 49.8 mg (0.01 mmol, 1 eq) of Fmoc-(Lys-Gly-Trp-Gly-Trp-Gly)₅-NH₂ (SEQ ID NO:80), was dissolved in 0.5 mL of dry DMSO. To this solution was added 0.492 mL of Compound 53 (0.03 mmol, 2.5 eq) as a 40 mg/mL stock solution in dry DMSO. TEA (0.01 mmol, 1 eq) was added and the reaction mixture was stirred at room temperature for 4 hours. Analytical HPLC using a gradient of 45-65% acetonitrile/H2O (0.05% TFA) over 10 minutes showed complete conversion to the penta-substituted intermediate. The reaction was quenched by addition of amino-2-propanol (0.03 mmol, 2.5 eq) and then 0.5 mL of 20% piperidine in DMF was added and the reaction mixture was stirred at room temperature for 30 minutes. The reaction mixture was added to 50 mL of ether and centrifuged at 3000 g at 4° C. for 10 minutes. The product was collected as a solid pellet and then washed twice more with ether, followed by drying under vacuum to yield the deprotected intermediate. 73.4 mg of the deprotected intermediate (0.0131 mmol, 1 eq) was dissolved in 0.5 mL of dry DMSO, followed by the addition of 0.066 mL (0.0196 mmol, 1.5 eq) of DBCO-NHS (40 mg/mL) and TEA (0.0131 mmol, 1 eq). The reaction was stirred for 1 hour at room temperature and then quenched by the addition of amino-2-propanol (0.0196 mmol, 1.5 eq). The product was then precipitated from 50 mL of 1 M HCl and centrifuged at 3000 g at 4° C. for 10 minutes. The product was collected as a solid pellet and then washed once more with 1 M HCl and once more with DI water. The final collected pellet was dried under vacuum to yield 15.1 mg (26% yield) of the final product. MS (ESI) calculated for C₃₁₉H₃₉₆N₇₂O₄₂ m/z 5909.1 found 1183 (m/5)⁺.

### Example 6—Synthesis of Amphiphilic Block Copolymers Linked to Drug Molecules of Formula S-[B]-H(D)

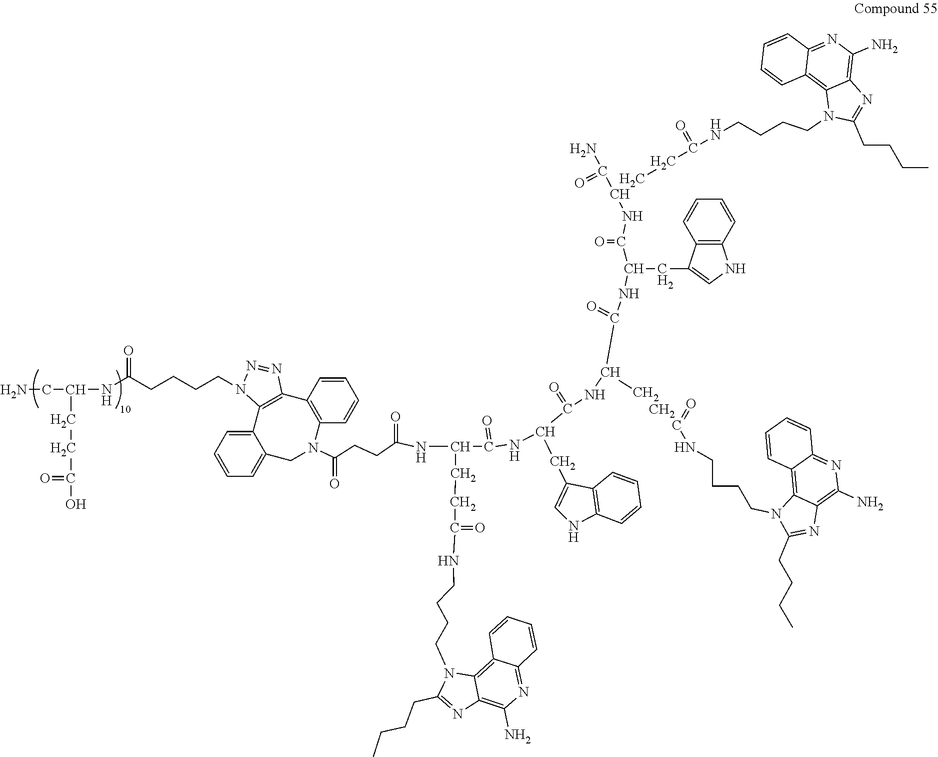

Compound 55, referred to as E₁₀-2B₃W₂, was synthesized using Azido-(Glu)₁₀-NH₂ (SEQ ID NO:81) and Compound 42 as the starting materials. 5 mg of Azido-(Glu)₁₀-NH₂ (SEQ ID NO: 81), (0.0035 mmol, 1 eq) was dissolved in dry DMSO and 6.77 mg of Compound 42 (0.0035 mmol, 1 eq) as a 40 mg/mL solution in dry DMSO was added. The reaction mixture was stirred overnight at room temperature. Compound 55 was purified on a preparatory HPLC system using a gradient of 25-45% acetonitrile/H₂O (0.05% TFA) over 10 minutes on an Agilent Prep-C18 column, 30×100 mm, 5 μm. The resulting fractions were collected, frozen and then lyophilized to obtain 11.8 mg of a spectroscopically pure (>95% AUC at 254 nm) white powder in quantitative yield. MS (ESI) calculated for m/z 3377.31, found 1127 (M/3)⁺.

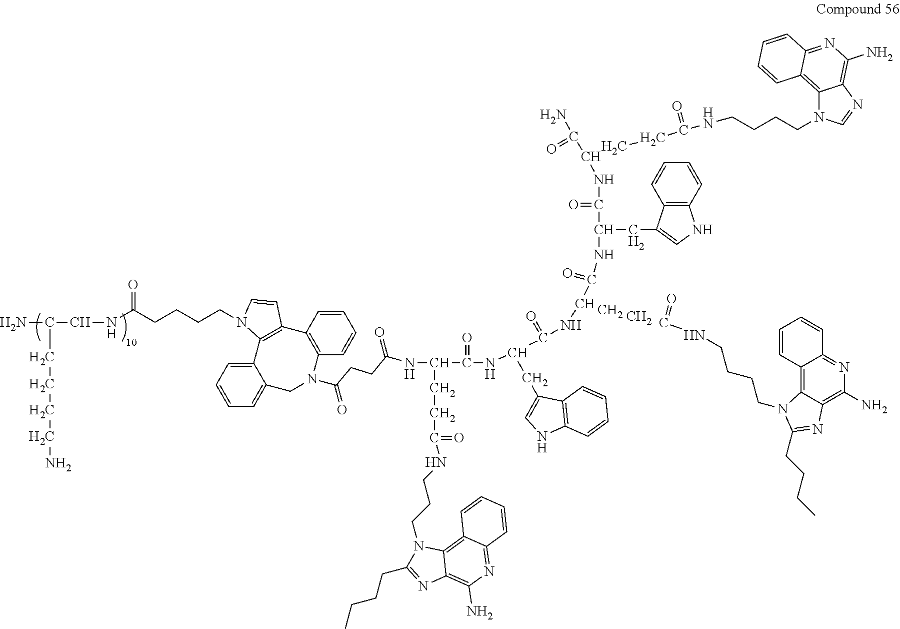

Compound 56, referred to as K₁₀-2B₃W₂ was synthesized using the same procedure as Compound 55, except Azido-(Lys)₁₀-NH₂ (SEQ ID NO:82), was used as the starting material. Compound 46 was purified on a preparatory HPLC system using a gradient of 20-40% acetonitrile/H₂ (0.05% TFA) over 10 minutes on an Agilent Prep-C18 column, 30×100 mm, 5 μm. The resulting fractions were collected, frozen and then lyophilized to obtain a spectroscopically pure (>95% AUC at 254 nm) white powder in quantitative yield. MS (ES calculated for m/z 3367.58, found 482 (M/7)r.

### Example 7—Formulation of Mosaic Particles Comprising S-B-H+D-H

Immunostimulants and antineoplastic (or “chemotherapeutic”) compounds are often highly hydrophobic and require the use of a delivery system to solubilize the drug molecule (D). Moreover, unformulated immunostimulants and chemotherapeutic compounds can broadly distribute following administration to a subject, and this can lead to dose-limiting toxicities. Therefore, drug delivery systems are needed to improve drug solubility and to prevent systemic distribution, which is a major cause of drug molecule (D) toxicity.

To evaluate the potential of the amphiphilic block copolymers described herein as drug carriers for cancer treatment, two types of drug molecules for evaluated for incorporation into amphiphilic block copolymers of formula S-B-H.

To 1 mg of compound 30 at 20 mg/mL DMSO was added 1 mg of compound 37 at 20 mg/mL in DMSO, followed by vigorous mixing. To evaluate particle size and stability, 10 μL of the resulting mixture was diluted with 190 μL PBS, pH 7.4 and the resulting solution was evaluated for turbidity and by dynamic light scattering (DLS). No aggregation was observed by turbidity measurements and DLS revealed that the mosaic particles comprising a TLR-7/8 agonist, compound 37, were 28.76 nm in diameter.

To 1 mg of compound 30 at 20 mg/mL DMSO was added 1 mg of compound 38 at 20 mg/mL in DMSO, followed by vigorous mixing. To evaluate particle size and stability, 10 μL of the resulting mixture was diluted with 190 μL PBS, pH 7.4 and the resulting solution was evaluated for turbidity and by DLS. No aggregation was observed by turbidity measurements and DLS revealed that the mosaic particles comprising an anthracycline anti-neoplastic compound, compound 38, were 19.68 nm in diameter.

These results indicate that amphiphilic block polymers comprising a charged molecule (C) and poly(amino acid)-based hydrophobic polymers or oligomers (H) further comprising aromatic amines permit high loading (up to 50% by mass) of a variety of different drug molecules (D) with anti-cancer properties in stable nanoparticles via a simple formulation process, i.e., simply mixing in a water-miscible organic solvent and then diluting with buffer.

### Example 8—Mosaic Particle Comprising S-B-H+(1) D-H+(2) D-H

It may be beneficial to combine drug molecules (D) with orthogonal mechanisms of action into the same particle as a means to augment treatment efficacy. In this regard, it may be beneficial to combine anthracyclines, which cause immunogenic cell death, with immuno-stimulants, which can induce anti-cancer T cell immunity.

Therefore, we assessed the capacity of the amphiphilic block polymers to incorporate both an anthracycline and an imidazoquinoline, TLR-7/8 agonist into the same nanoparticle micelle. To 1 mg of compound 30 at 20 mg/mL DMSO was added 1 mg of compound 37 at 20 mg/mL DMSO and 1 mg of compound 38 at 20 mg/mL in DMSO, followed by vigorous mixing. To evaluate particle size and stability, 10 μL of the resulting mixture was diluted with 190 μL PBS, pH 7.4 and the resulting solution was evaluated for turbidity and by DLS. No aggregation was observed by turbidity measurements and DLS revealed that the mosaic particles were 91.52 nm in diameter, indicating that the mosaic particles formed by amphiphilic block copolymers and multiple drug molecule hydrophobic polymers or oligomers conjugates (D-H) are stable with high drug loading efficiency.

### Example 9—Additional Hydrophobic Polymer or Oligomer (H) and Amphiphilic Block Copolymer Compositions

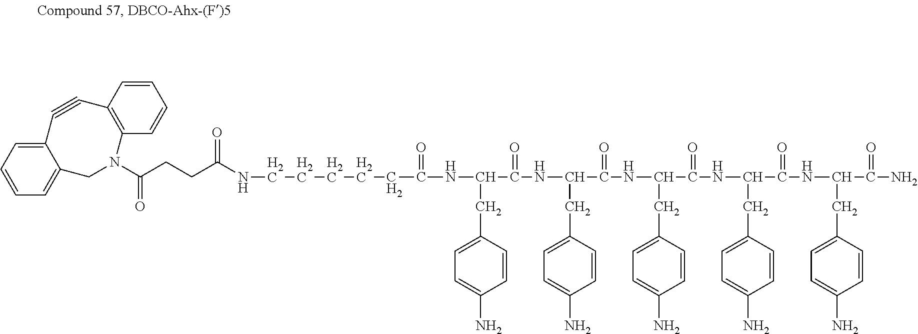 [SMILES: NC(=O)C(Cc1ccc(N)cc1)NC(=O)C(Cc1ccc(N)cc1)NC(=O)C(Cc1ccc(N)cc1)NC(=O)C(Cc1ccc(N)cc1)NC(=O)C(Cc1ccc(N)cc1)NC(=O)CCCCCNC(=O)CCC(=O)N1Cc2ccccc2C#Cc2ccccc21]

Compound 57, referred to as DBCO-Ahx-F′₅ or Ahx-F′₅ was synthesized by reacting 400 mg (0.4 mmol, 1 eq) of the precursor Ahx-(F′)₅—NH₂, which was prepared by solid phase peptide synthesis, with 171.05 mg of DBCO-NHS (0.4 mmol, 1.0 eq) and 258.1 mg of Triethylamine (2.55 mmol, 6.0 eq) in 3.7 mL of DMSO. The DBCO-NHS was added in 4 increments of 0.25 eq. The reaction was run overnight at room temperature and HPLC indicated that the reaction was complete by 24 hours. Compound 57 was purified on a preparatory HPLC system using a gradient of 13-43% acetonitrile/H₂O (0.05% TFA) over 12 minutes on an Agilent Prep-C18 column, 50×100 mm, 5 μm. The product eluted at ˜5.7 minutes and the resulting fractions were collected, frozen and then lyophilized to obtain 217.0 mg (41.5% yield) of a spectroscopically pure (>95% AUC at 254 nm) white/yellow powder. MS (ESI) calculated for C₇₀H₇₆N₁₂O₉ m/z 1228.59, found 1228.7 (M+H)⁺.

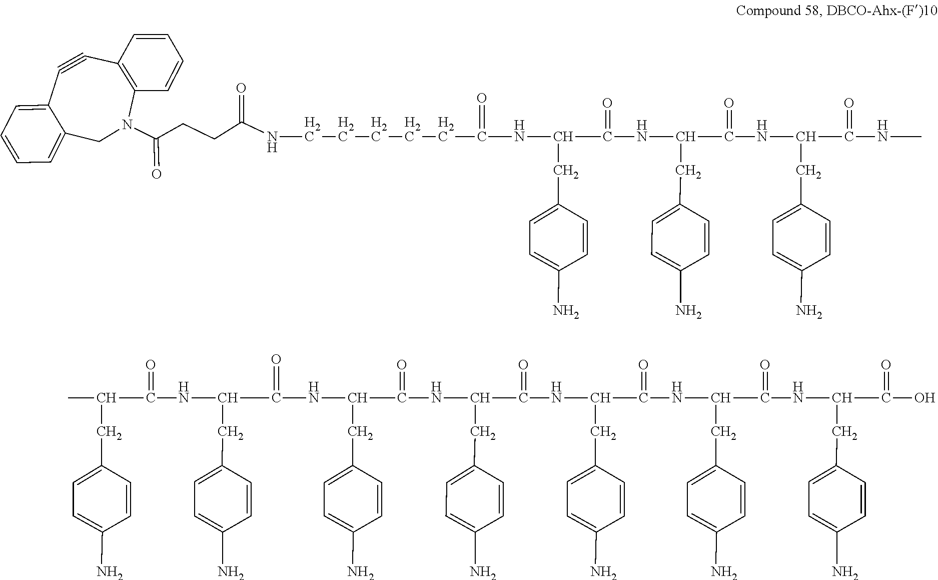

Compound 58, referred to as DBCO-Ahx-F′₁₀ or Ahx-F′₁₀ was synthesized by reacting 450 mg (0.26 mmol, 1 eq) of the precursor Ahx-(F′)₁₀—NH₂ (SEQ ID NO:83), which was prepared by solid phase peptide synthesis, with 103.4 mg of DBCO-NHS (0.26 mmol, 1.0 eq) and 286.1 mg of Triethylamine (2.83 mmol, 11.0 eq) in 3.3 mL of DMSO. The DBCO-NHS was added in 4 increments of 0.25 eq. The reaction was run overnight at room temperature and HPLC indicated that the reaction was complete by 24 hours. Compound 58 was purified on a preparatory HPLC system using a gradient of 15-45% acetonitrile/H₂O (0.05% TFA) over 12 minutes on an Agilent Prep-C18 column, 50×100 mm, 5 μm. The product eluted at −5.1 minutes and the resulting fractions were collected, frozen and then lyophilized to obtain 265.4 mg (50.6% yield) of a spectroscopically pure (>95% AUC at 254 nm) red/copper powder. MS (ESI) calculated for C₂₀₅H₂₂₆N₄₂O₂₄ m/z 3659.78, found 1221.3 (M+3H)⁺.

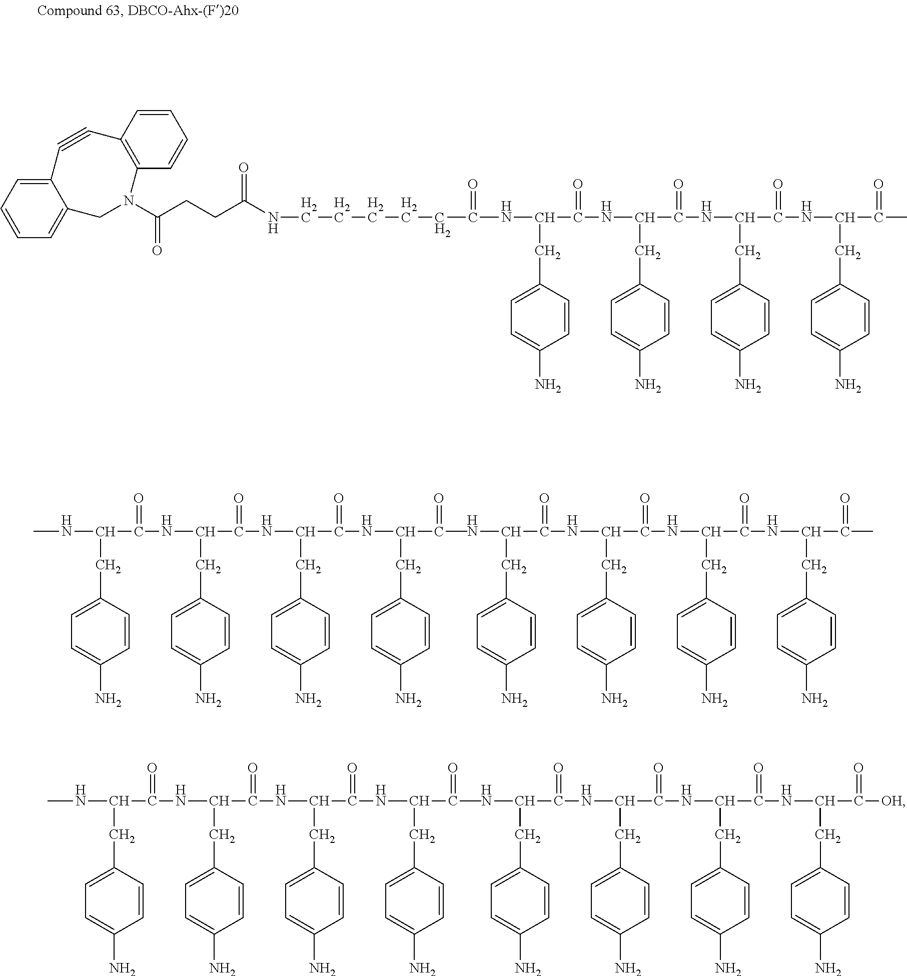

Compound 59, referred to as DBCO-Ahx-F′₂₀ or Ahx-F′₂₀ DBCO-Ahx-(F′)20 (SEQ ID NO:84) was synthesized by reacting 480 mg (0.14 mmol, 1 eq) of the precursor Ahx-(F′)₂₀—NH₂ (SEQ ID NO:85), which was prepared by solid phase peptide synthesis, with 57.3 mg of DBCO-NHS (0.14 mmol, 1.0 eq) and 302.4 mg of Triethylamine (2.99 mmol, 21.0 eq) in 3.0 mL of DMSO. The DBCO-NHS was added in 4 increments of 0.25 eq. The reaction was run overnight at room temperature and HPLC indicated that the reaction was complete by 24 hours. Compound 59 was purified on a preparatory HPLC system using a gradient of 13-43% acetonitrile/H₂O (0.05% TFA) over 12 minutes on an Agilent Prep-C18 column, 50×100 mm, 5 μm. The product eluted at ˜5.5 minutes and the resulting fractions were collected, frozen and then lyophilized to obtain 106.6 mg (20.5% yield) of a spectroscopically pure (94.4% AUC at 254 nm) brown/copper powder. MS (ESI) calculated for C₁₁₅H₁₂₆N₂₂O₁₄ m/z 2039.99, found 1020.5 (M+2H)⁺.

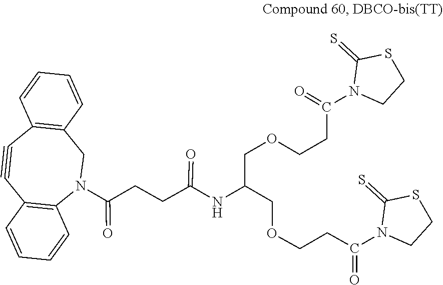 [SMILES: O=C(CCC(=O)N1Cc2ccccc2C#Cc2ccccc21)NC(COCCC(=O)N1CCSC1=S)COCCC(=O)N1CCSC1=S]

Compound 60, referred to as DBCO-2-Amino-1,3-bis(carboxylethoxy)propane(TT)2 or DBCO-bis(TT) was synthesized by reacting 385.6 mg (0.74 mmol, 1 eq) of the precursor DBCO-2-Amino-1,3-bis(carboxylethoxy)propane, with 193.4 mg of 2-Thiazoline-2-thiol (1.62 mmol, 2.2 eq) and 367.5 mg of 1-Ethyl-3-(3-dimethylaminopropyl) carbodiimide (1.92 mmol, 2.6 eq) in and 4-Dimethylaminopyridine in 4.0 mL of DCM. The reaction was run overnight at room temperature and HPLC indicated that the reaction was complete by 24 hours. The product eluted at 6.8 minutes on an Agilent analytical C18 column, 4.6×100 mm, 2.7 μm. Compound 60 was extracted with ethyl acetate and 1M HCl and was dried on the rotovap to obtain 317.1 mg (59.3% yield) of an impure (27.0% AUC at 254 nm) yellow powder. MS (ESI) calculated for C₃₄H₃₆N₄O₆S₄ m/z 724.15, found 725.3 (M+H)⁺

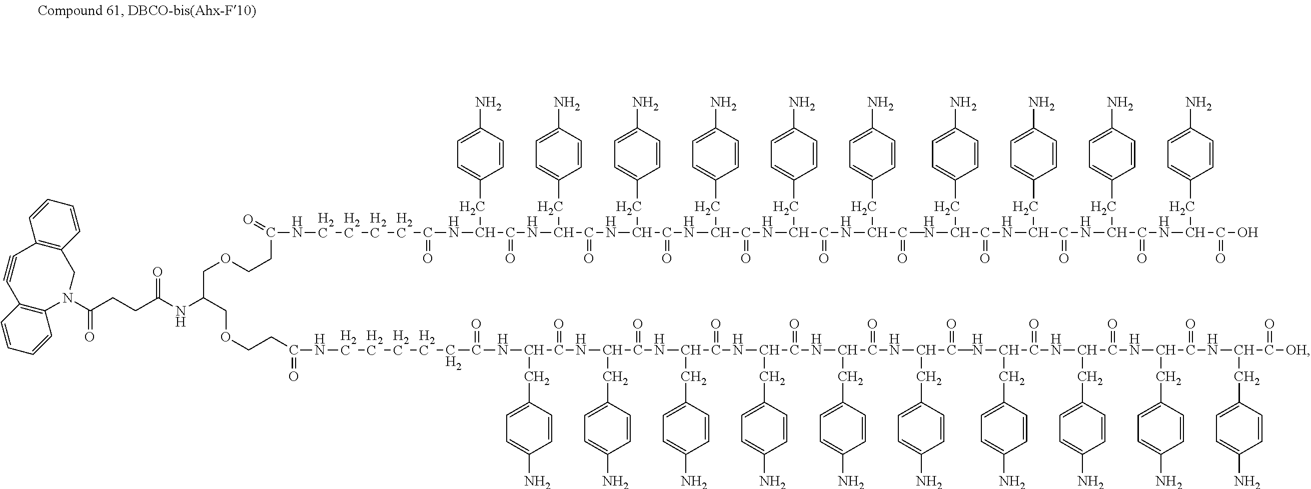

Compound 61, referred to as DBCO-2-Amino-1,3-bis(carboxylethoxy)propane(Ahx-F′10)2 or DBCO-bis(Ahx-F′10) was synthesized by reacting 13.0 mg (0.018 mmol, 1 eq) of the precursor DBCO-2-Amino-1,3-bis(carboxylethoxy)propane(TT)2, Compound 61, with 314.2 mg of Ahx-(F′)₁₀—NH₂ (SEQ ID NO:87) (0.18 mmol, 10 eq) that was prepared by solid phase peptide synthesis and 199.5 mg of Triethylamine (1.97 mmol, 11.0 eq) in 1.8 mL of DMSO. The reaction was run overnight at room temperature and HPLC indicated that the reaction was complete by 24 hours. Compound 61 was purified on a preparatory HPLC system using a gradient of 5-25-35% acetonitrile/H₂O (0.05% TFA) over 14 minutes on an Agilent Prep-C18 column, 50×100 mm, 5 μm. The product eluted at ˜9.8 minutes and the resulting fractions were collected, frozen and then lyophilized to obtain 19.16 mg (26.8% yield) of a spectroscopically pure (83.4% AUC at 254 nm) orange powder. MS (ESI) calculated for C₂₂₀H₂₅₂N₄O₃₀ m/z 3989.95, found 1330.8 (M+3H)⁺.

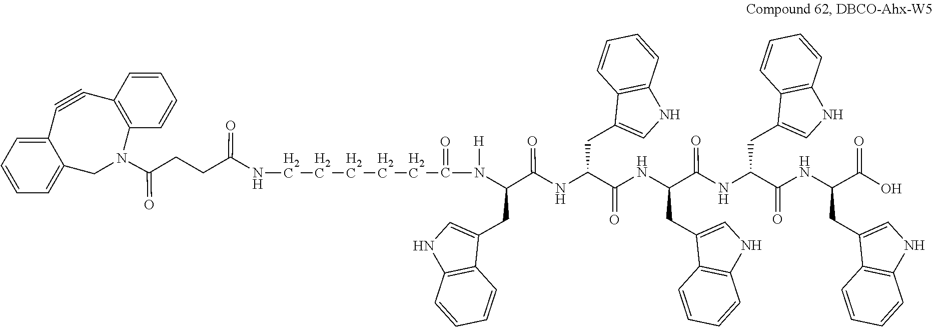 [SMILES: O=C(CCC(=O)N1Cc2ccccc2C#Cc2ccccc21)NCCCCCC(=O)N[C@H](Cc1c[nH]c2ccccc12)C(=O)N[C@H](Cc1c[nH]c2ccccc12)C(=O)N[C@H](Cc1c[nH]c2ccccc12)C(=O)N[C@H](Cc1c[nH]c2ccccc12)C(=O)N[C@H](Cc1c[nH]c2ccccc12)C(=O)O]

Compound 62, referred to as DBCO-Ahx-W5 was synthesized by reacting 14.2 mg (0.035 mmol, 1 eq) of the precursor DBCO-NHS, with 37.5 mg of Ahx-(W)₅—NH₂ (SEQ ID NO:88) (0.035 mmol, 1 eq) that was prepared by solid phase peptide synthesis and 3.93 mg of Triethylamine (0.039 mmol, 1.1 eq) in 0.5 mL of DMSO. The reaction was run overnight at room temperature and HPLC indicated that the reaction was complete by 24 hours. Compound 62 was crashed out in twice 1M HCL and once in H2O to obtain 34.3 (71.9% yield) of a spectroscopically pure (92.6% AUC at 254 nm) pink powder. MS (ESI) calculated for C₈₀H₇₆N₁₂O₉ m/z 1348.59, found 1348.4 (M+H)⁺.

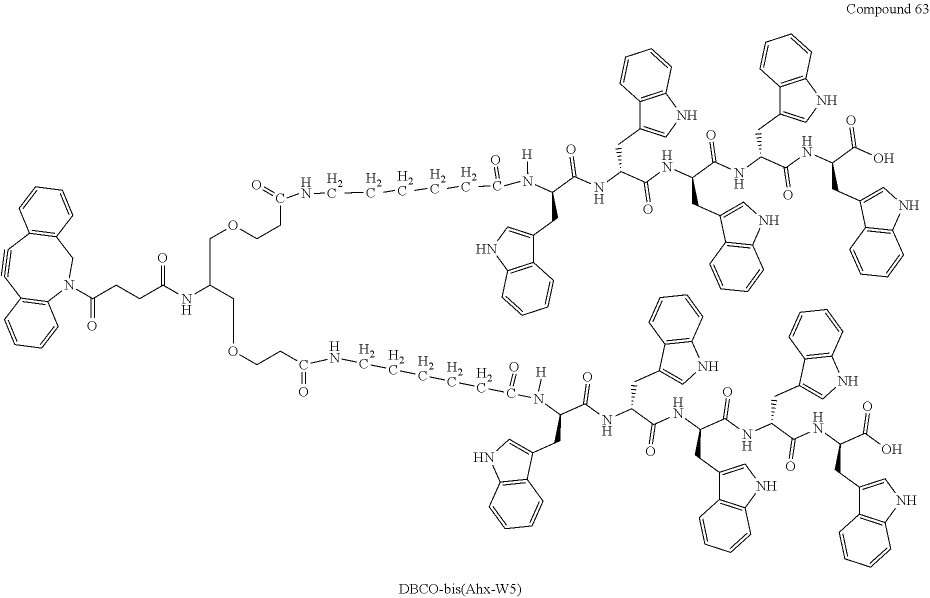 [SMILES: O=C(CCOCC(COCCC(=O)NCCCCCC(=O)N[C@H](Cc1c[nH]c2ccccc12)C(=O)N[C@H](Cc1c[nH]c2ccccc12)C(=O)N[C@H](Cc1c[nH]c2ccccc12)C(=O)N[C@H](Cc1c[nH]c2ccccc12)C(=O)N[C@H](Cc1c[nH]c2ccccc12)C(=O)O)NC(=O)CCC(=O)N1Cc2ccccc2C#Cc2ccccc21)NCCCCCC(=O)N[C@H](Cc1c[nH]c2ccccc12)C(=O)N[C@H](Cc1c[nH]c2ccccc12)C(=O)N[C@H](Cc1c[nH]c2ccccc12)C(=O)N[C@H](Cc1c[nH]c2ccccc12)C(=O)N[C@H](Cc1c[nH]c2ccccc12)C(=O)O]

Compound 63, referred to as DBCO-2-Amino-1,3-bis(carboxylethoxy)propane(Ahx-W5)2 or DBCO-bis-(Ahx-W5) was synthesized by reacting 13.0 mg (0.018 mmol, 1 eq) of the precursor DBCO-2-Amino-1,3-bis(carboxylethoxy)propane(TT)2, with 41.3 mg of Ahx-(W)₅—NH₂ (0.039 mmol, 2.2 eq) that was prepared by solid phase peptide synthesis and 9.1 mg of Triethylamine (0.09 mmol, 2.3 eq) in 0.3 mL of DMSO. The reaction was run overnight at room temperature and HPLC indicated that the reaction was complete by 24 hours. Compound 63 was purified on a preparatory HPLC system using a gradient of 15-60-90% acetonitrile/H₂O (0.05% TFA) over 16 minutes on an Agilent Prep-C18 column, 30×100 mm, 5 μm. The product eluted at ˜12.7 minutes and the resulting fractions were collected, frozen and then lyophilized to obtain 12.5 mg (30.8% yield) of a spectroscopically pure (>95% AUC at 254 nm) pink powder. MS (ESI) calculated for C₁₅₀H₁₅₂N₂₄O₂₀ m/z 2609.16, found 1305.0 (M+2H)⁺.

### Example 10—Synthesis of Amphiphilic Block Copolymers of Formula (S-[B])-H(D), Wherein the Hydrophobic Polymer or Oligomer (H) is Branched

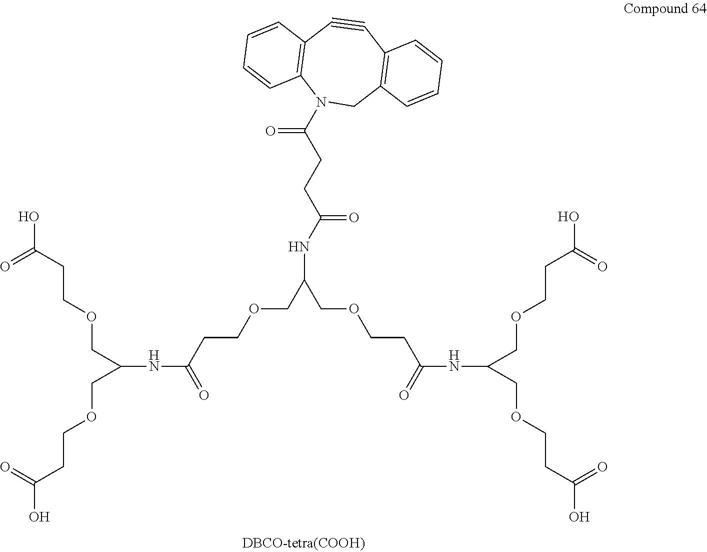 [SMILES: O=C(O)CCOCC(COCCC(=O)O)NC(=O)CCOCC(COCCC(=O)NC(COCCC(=O)O)COCCC(=O)O)NC(=O)CCC(=O)N1Cc2ccccc2C#Cc2ccccc21]

Compound 64, referred to as DBCO-2-Amino-1,3-bis(carboxylethoxy)propane(COOH)₄ or DBCO-tetra(COOH), was synthesized by reacting 250 mg (0.34 mmol, 1.1 eq) of the precursor Compound 60 with 170 mg of DBCO-2-Amino-1,3-bis(carboxylethoxy)propane (0.6 mmol, 2 eq) and 190 mg of TEA (1.9 mmol, 6 eq) in 2.5 mL of DMF. The reaction was run for 1 hour at room temperature and HPLC indicated the reaction was complete. MS (ESI) calculated for C₄₆H₆₀N₄O₁₈ m/z 956.4, found 957.2 (M+H)⁺.

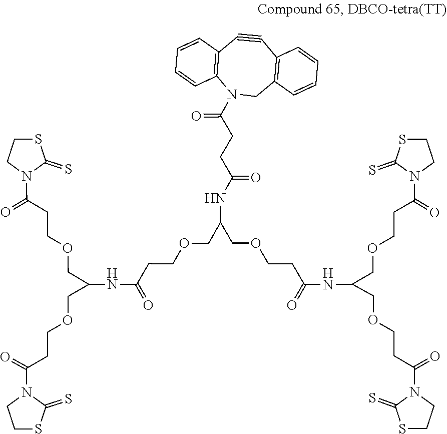 [SMILES: O=C(CCOCC(COCCC(=O)NC(COCCC(=O)N1CCSC1=S)COCCC(=O)N1CCSC1=S)NC(=O)CCC(=O)N1Cc2ccccc2C#Cc2ccccc21)NC(COCCC(=O)N1CCSC1=S)COCCC(=O)N1CCSC1=S]

Compound 65, referred to as DBCO-2-Amino-1,3-bis(carboxylethoxy)propane(TT)₄ or DBCO-tetra(TT), was synthesized by reacting 178 mg (0.19 mmol, 1 eq) of the precursor Compound 64 with 115 mg of 2-Thiazoline-2-thiol (0.96 mmol, 5.2 eq). TEA (2.98 mmol, 16 eq) was added and the reaction mixture was cooled in an ice bath for 5 minutes. 310 mg of HATU (0.8 mmol, 4.4 eq) was added and the reaction mixture was stirred in an ice bath. The progress of the reaction was monitored by LC-MS. After 2 hours, the reaction was complete. Compound 65 was crashed out once in 1M HCl and once in H₂O. The resulting solid was dissolved in ACN and dried on rotovap to obtain 215 mg (85.0% yield) of an impure (53.0% AUC at 254 nm) yellow/brown oil. MS (ESI) calculated for C₅₈H₇₂N₈O₁₄S₈ m/z 1360.3, found 1361.0 (M+H)⁺.

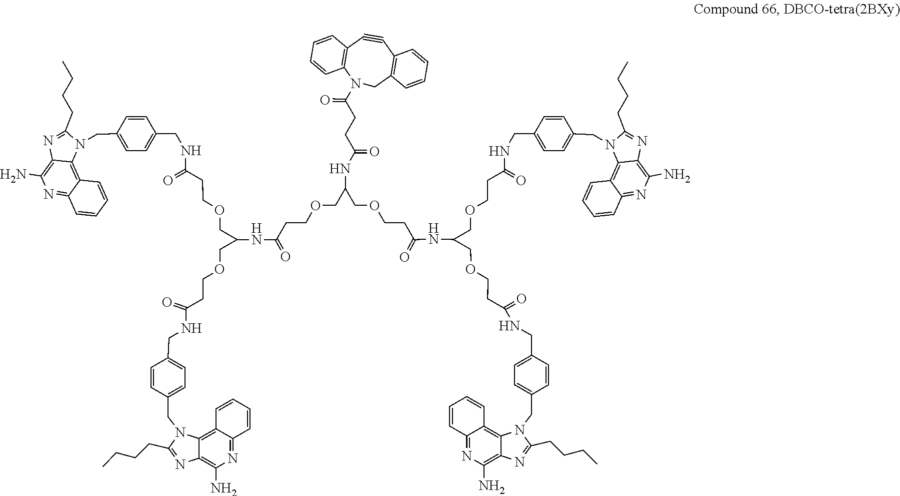 [SMILES: CCCCc1nc2c(N)nc3ccccc3c2n1Cc1ccc(CNC(=O)CCOCC(COCCC(=O)NCc2ccc(Cn3c(CCCC)nc4c(N)nc5ccccc5c43)cc2)NC(=O)CCOCC(COCCC(=O)NC(COCCC(=O)NCc2ccc(Cn3c(CCCC)nc4c(N)nc5ccccc5c43)cc2)COCCC(=O)NCc2ccc(Cn3c(CCCC)nc4c(N)nc5ccccc5c43)cc2)NC(=O)CCC(=O)N2Cc3ccccc3C#Cc3ccccc32)cc1]

Compound 66, referred to as DBCO-2-Amino-1,3-bis(carboxylethoxy)propane(2BXy)₄ or DBCO-tetra(2BXy), was synthesized by reacting 16 mg (0.012 mmol, 1 eq) of the precursor Compound 65 with 17 mg of Compound 36 (0.047 mmol, 4 eq) and TEA (0.047 mmol, 4 eq) in 0.5 mL DMSO. The progress of the reaction was monitored by HPLC. After 1 hour, the reaction was complete. Compound 66 was purified on a preparatory HPLC system using a gradient of 38-48% acetonitrile/H₂O (0.05% TFA) over 12 minutes on an Agilent Prep-C18 column, 30×100 mm, 5 μm. The product eluted at ˜4.0 minutes and the resulting fractions were collected, frozen and then lyophilized to obtain 13.6 mg (49.8% yield) of a spectroscopically pure (98.3% AUC at 254 nm) white powder. MS (ESI) calculated for C₁₃₄H₁₅₂N₂₄O₁₄ m/z 2321.2, found 775.0 (M/3+H)⁺.

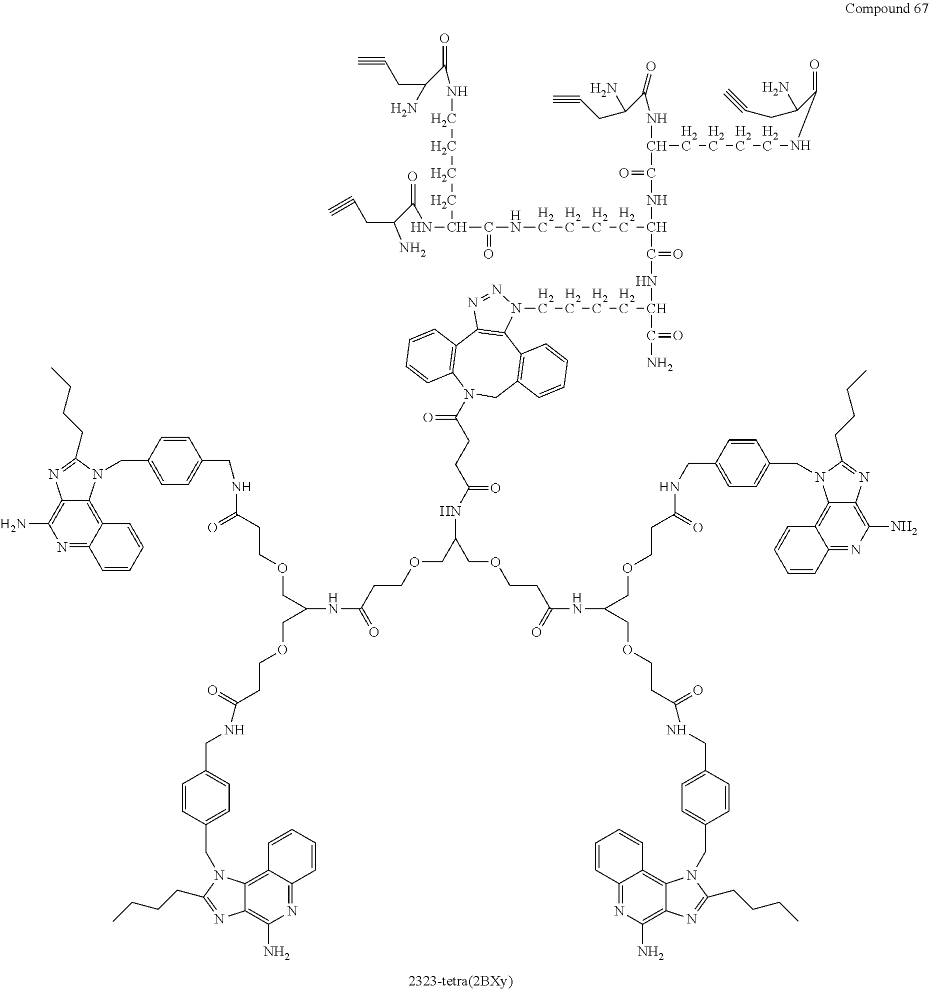 [SMILES: C#CCC(N)C(=O)NCCCCC(NC(=O)C(N)CC#C)C(=O)NCCCCC(NC(=O)C(CCCCNC(=O)C(N)CC#C)NC(=O)C(N)CC#C)C(=O)NC(CCCCn1nnc2c1-c1ccccc1CN(C(=O)CCC(=O)NC(COCCC(=O)NC(COCCC(=O)NCc1ccc(Cn3c(CCCC)nc4c(N)nc5ccccc5c43)cc1)COCCC(=O)NCc1ccc(Cn3c(CCCC)nc4c(N)nc5ccccc5c43)cc1)COCCC(=O)NC(COCCC(=O)NCc1ccc(Cn3c(CCCC)nc4c(N)nc5ccccc5c43)cc1)COCCC(=O)NCc1ccc(Cn3c(CCCC)nc4c(N)nc5ccccc5c43)cc1)c1ccccc1-2)C(N)=O]

Compound 67, referred to as {propargyl}₄K2K{Lys(N₃)}-DBCO-2-Amino-1,3-bis(carboxylethoxy)propane(2BXy)₄ or 2323-tetra(2BXy), was synthesized by reacting 16 mg of {propargyl}₄K2K{Lys(N₃)}, which was prepared by solid phase peptide synthesis, (0.17 mmol, 1 eq) and 39 mg of Compound 66 (0.017 mmol, 1 eq) in 1.0 mL dry DMSO. The reaction mixture was stirred overnight at room temperature. HPLC indicated that the reaction was complete. Compound 67 was purified on a preparatory HPLC system using a gradient of 20-50% acetonitrile/H₂O (0.05% TFA) over 12 minutes on an Agilent Prep-C18 column, 30×100 mm, 5 m. The product eluted at ˜6.5 minutes and the resulting fractions were collected, frozen and then lyophilized to obtain 26 mg (47.5% yield) of a spectroscopically pure (98.6% AUC at 254 nm) white powder. MS (ESI) calculated for C₁₇₈H₂₂₁N₃₉O₂₂ m/z 3256.7, found 1087.0 (M/3+H)⁺.

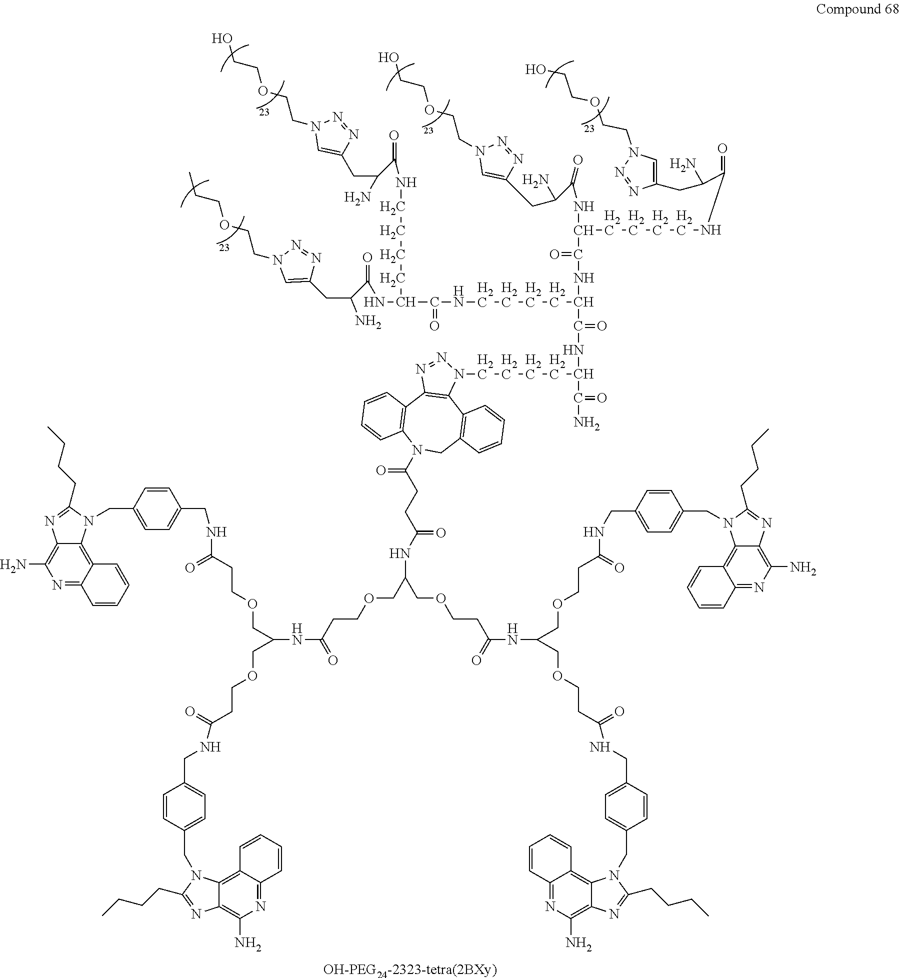

Compound 68, referred to as {OH-PEG₂₄}4-{propargyl}₄K2K{Lys(N₃)}-DBCO-2-Amino-1,3-bis(carboxylethoxy)propane(2BXy)₄ or OH-PEG₂₄-2323-tetra(2BXy), was synthesized by reacting 3.5 mg Compound 67 (0.0011 mmol, 1 eq) with 4.7 mg of alpha-Azido-omega-hydroxy 24 (ethylene glycol) (0.0043 mmol, 4 eq) in 0.058 mL H₂O and 0.117 mL DMSO. To the reaction mixture, 0.8 mg Sodium Ascorbate (0.0043 mmol and 4 eq) was added. 1.1 mg Copper Sulfate Pentahydrate (0.0043 mmol, 4 eq) and 1.9 mg tris-hydroxypropyltriazolylmethylamine (0.0043 mmol, 4 eq) were combined in a separate vial, and then added to the reaction mixture. The reaction mixture was stirred overnight at room temperature. LC-MS indicated that the reaction was complete. Compound 68 was purified by dialysis with a Regenerated Cellulose membrane, MWCO: 2 kDa, with solvent changes of 1:1 H₂O/MeOH with 0.01% EDTA (2×), 1:1 H₂O/MeOH (1×), and MeOH (2×). Sample collected and dried on rotovap to obtain 5.8 mg (70.5% yield) of a spectroscopically pure (99.3% AUC at 254 nm) blue solid. MS (ESI) calculated for C₃₇₀H₆₀₉N₅₁O₁₁₈ m/z 7655.3, found 1277.8 (M/6)⁺.

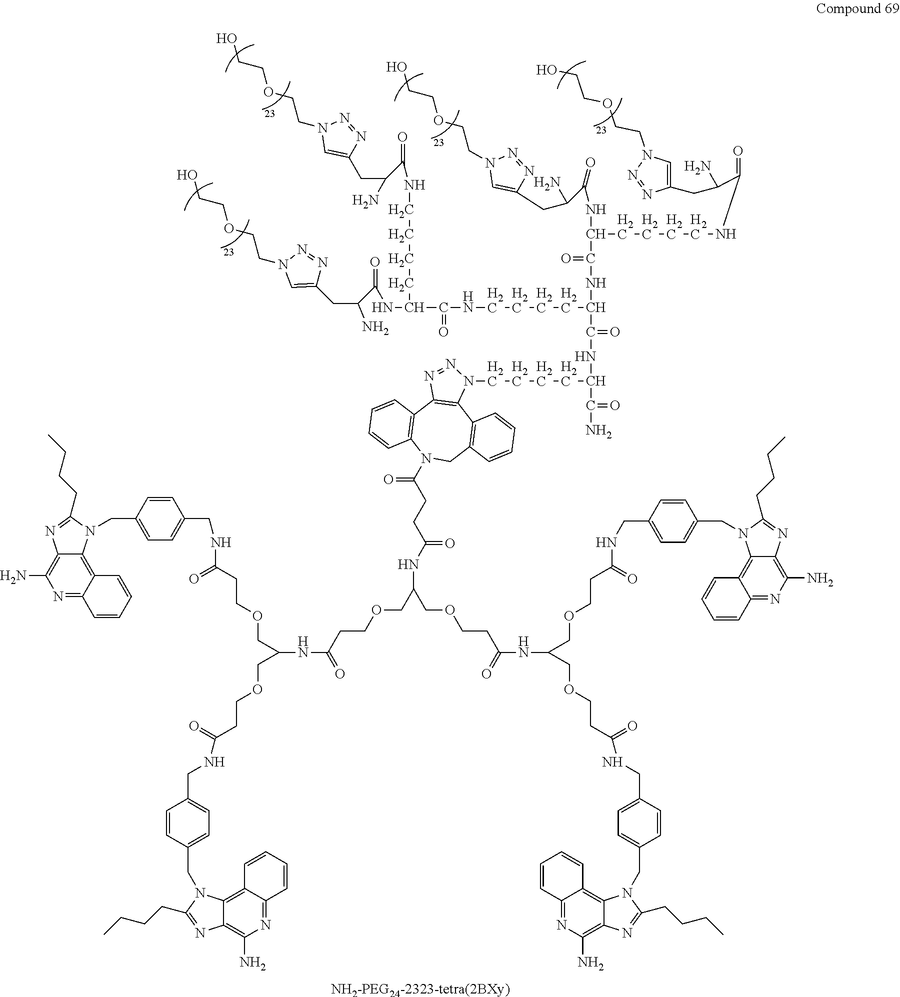

Compound 69, referred to as {NH₂—PEG₂₄}4-{propargy}₄K2K{Lys(N₃)}-DBCO-2-Amino-1,3-bis(carboxylethoxy)propane(2BXy)₄ or NH₂—PEG2₄-2323-tetra(2BXy), was synthesized and purified using the same procedure as Compound 68, except alpha-Azido-omega-amino 23 (ethylene glycol) was used as the starting material. Upon collecting purified sample and drying on rotovap, 4.8 mg (58.4% yield) of a spectroscopically pure (95.5% AUC at 254 nm) green solid was obtained. MS (ESI) calculated for C₃₇₀H₆₁₃N₅₅O₁₁₄ m/z 7651.4, found 1276.8 (M/6+H)⁺.

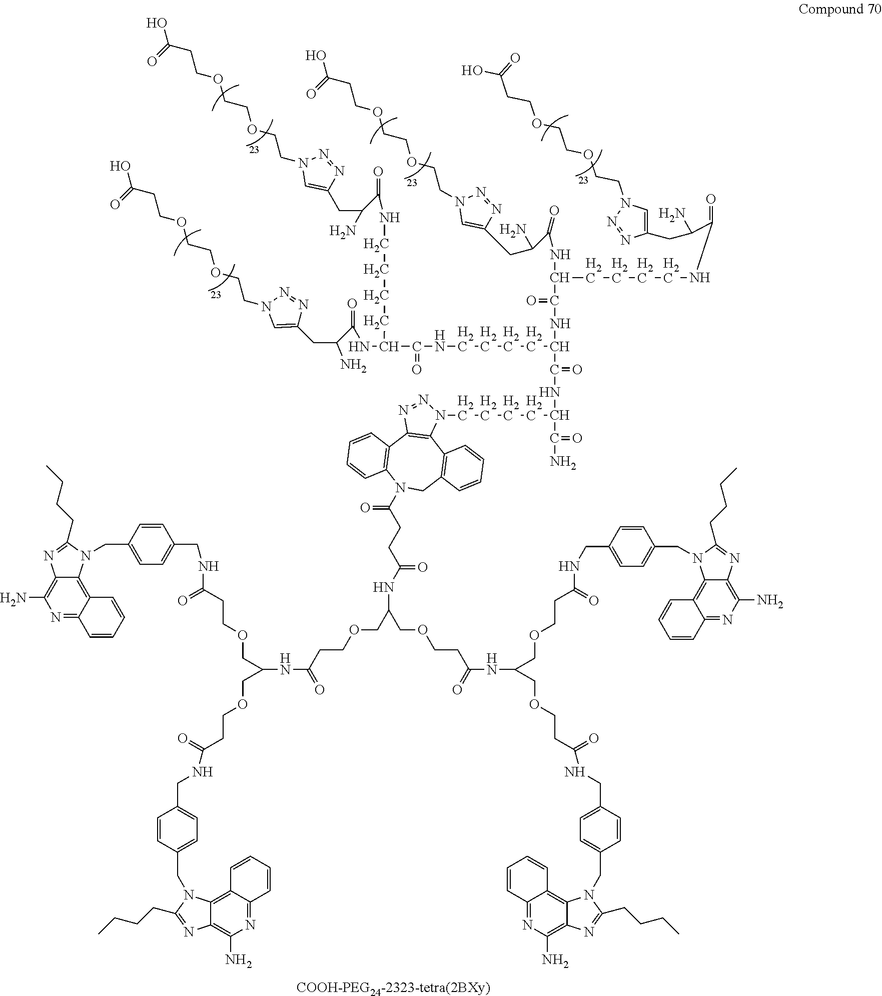

Compound 70, referred to as {COOH-PEG₂₄}4-{propargyl}₄K2K{Lys(N₃)}-DBCO-2-Amino-1,3-bis(carboxylethoxy)propane(2BXy)₄ or COOH-PEG₂₄-2323-tetra(2BXy), was synthesized and purified using the same procedure as Compound 68, except alpha-Azido-omega-(propionic acid) 24 (ethylene glycol) was used as the starting material. Upon collecting purified sample and drying on rotovap, 6.0 mg (70.3% yield) of a spectroscopically pure (98.0% AUC at 254 nm) blue solid was obtained. MS (ESI) calculated for C₃₈₂H₆₂₅N₅₁O₁₂₆ m/z 7943.1, found 1325.4 (M/6+H)⁺.

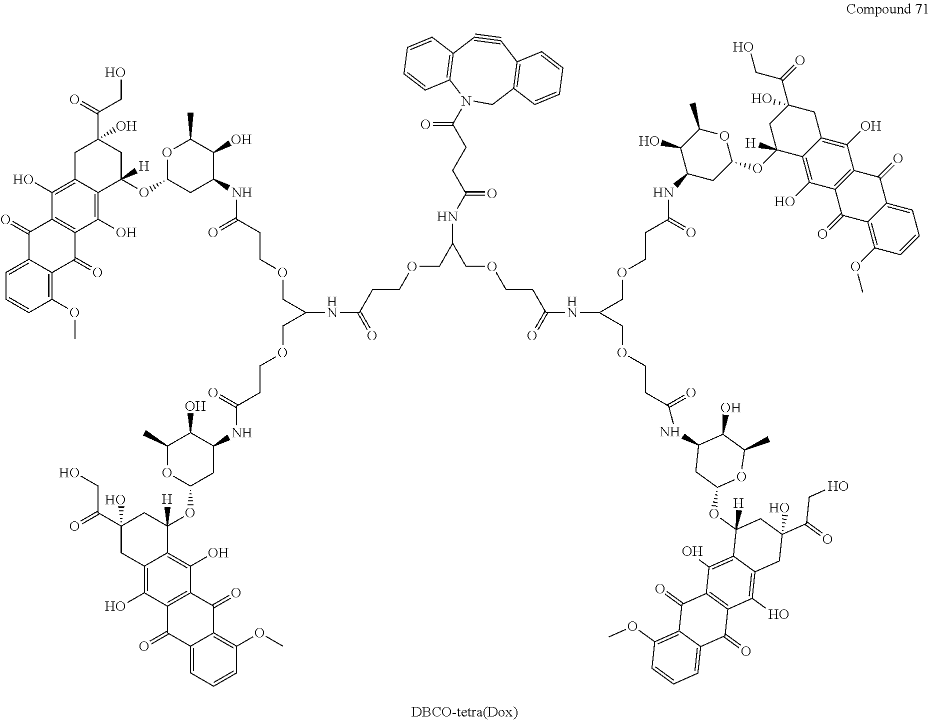 [SMILES: COc1cccc2c1C(=O)c1c(O)c3c(c(O)c1C2=O)C[C@@](O)(C(=O)CO)C[C@@H]3O[C@H]1C[C@H](NC(=O)CCOCC(COCCC(=O)N[C@H]2C[C@H](O[C@H]3C[C@](O)(C(=O)CO)Cc4c(O)c5c(c(O)c43)C(=O)c3c(OC)cccc3C5=O)O[C@@H](C)[C@H]2O)NC(=O)CCOCC(COCCC(=O)NC(COCCC(=O)N[C@@H]2C[C@@H](O[C@@H]3C[C@@](O)(C(=O)CO)Cc4c(O)c5c(c(O)c43)C(=O)c3c(OC)cccc3C5=O)O[C@H](C)[C@@H]2O)COCCC(=O)N[C@@H]2C[C@@H](O[C@@H]3C[C@@](O)(C(=O)CO)Cc4c(O)c5c(c(O)c43)C(=O)c3c(OC)cccc3C5=O)O[C@H](C)[C@@H]2O)NC(=O)CCC(=O)N2Cc3ccccc3C#Cc3ccccc32)[C@H](O)[C@H](C)O1]

Compound 72, referred to as DBCO-2-Amino-1,3-bis(carboxylethoxy)propane(Doxorubicin)₄ or DBCO-tetra(Dox), was synthesized by reacting 23 mg (0.017 mmol, 1 eq) of the precursor Compound 65 with 40 mg of Doxorubicin Hydrochloride (0.069 mmol, 4 eq) and TEA (0.138 mmol, 8 eq) in 1.5 mL DMSO. The progress of the reaction was monitored by HPLC. After 1 hour, the reaction was complete. Compound 72 was purified on a preparatory HPLC system using a gradient of 38-48% acetonitrile/H₂O (0.05% TFA) over 12 minutes on an Agilent Prep-C18 column, 30×100 mm, 5 μm. The product eluted at ˜7.5 minutes and the resulting fractions were collected, frozen and then lyophilized to obtain 5.3 mg (20.1% yield) of a spectroscopically pure (95.9% AUC at 254 nm) red powder. MS (ESI) calculated for C₁₅₄H₁₆₈N₈O₅₈ m/z 3059.0.

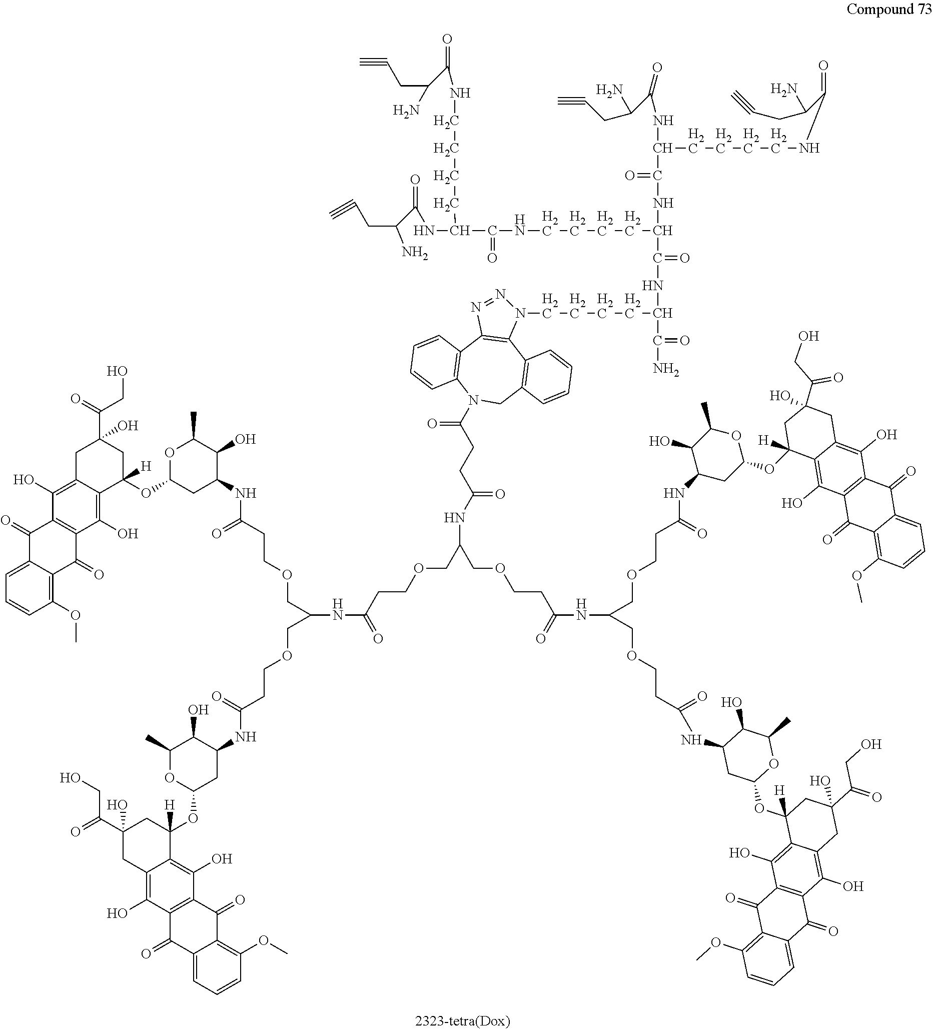

Compound 73, referred to as {propargyl}₄K2K{Lys(N₃)}-DBCO-2-Amino-1,3-bis(carboxylethoxy)propane(Doxorubicin)₄ or 2323-tetra(Dox), was synthesized by reacting 16 mg of {propargyl}₄K2K{Lys(N₃)} (0.17 mmol, 1 eq) and 53 mg of Compound 72 (0.017 mmol, 1 eq) in 2.0 mL dry DMSO. The reaction mixture was stirred overnight at room temperature. HPLC indicated that the reaction was complete. Compound 73 was purified on a preparatory HPLC system using a gradient of 25-45% acetonitrile/H₂O (0.05% TFA) over 12 minutes on an Agilent Prep-C18 column, 30×100 mm, 5 m. The product eluted at ˜4.6 minutes and the resulting fractions were collected, frozen and then lyophilized to obtain 28 mg (40.9% yield) of a spectroscopically pure (91.3% AUC at 254 nm) red/orange powder. MS (ESI) calculated for C₁₉₈H₂₃₇N₂₃O₆₆ m/z 3995.2, found 1332.8 (M/3+H)⁺.

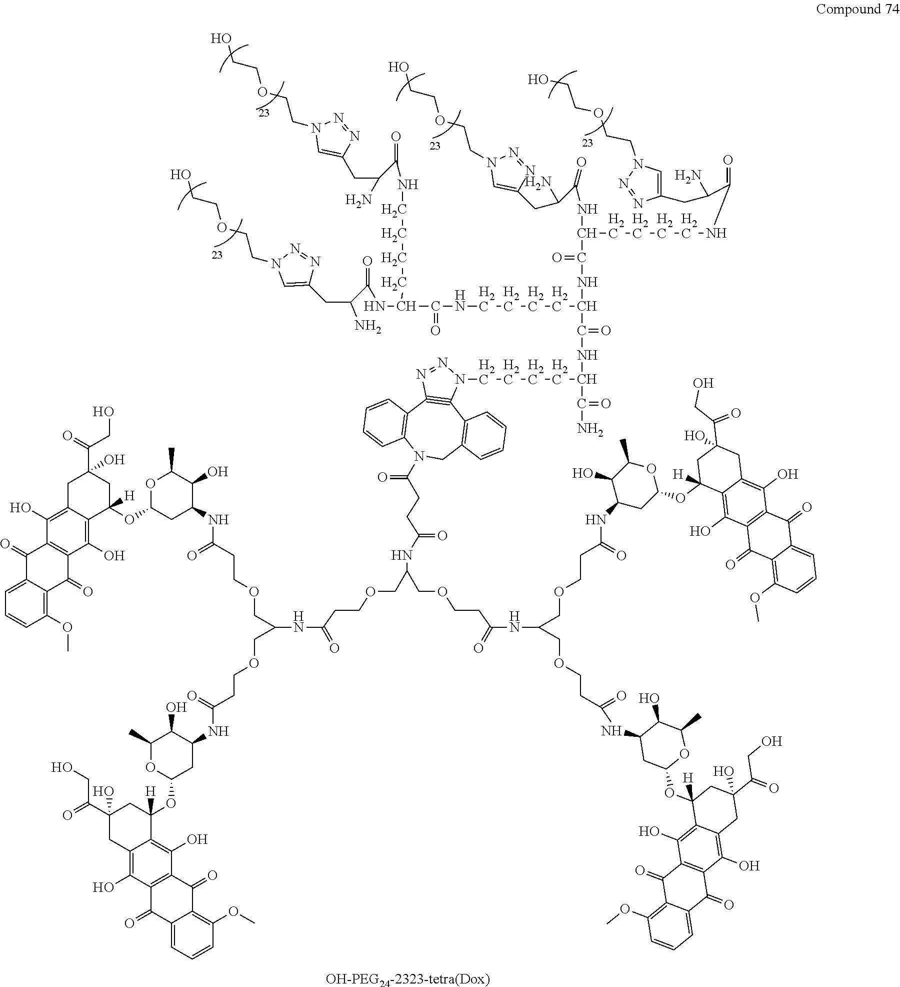

Compound 74, referred to as {OH-PEG₂₄}4-{propargyl}₄K2K{Lys(N₃)}-DBCO-2-Amino-1,3-bis(carboxylethoxy)propane(Doxorubucin)₄ or OH-PEG₂₄-2323-tetra(Dox), was synthesized by reacting 3.5 mg Compound 73 (0.0011 mmol, 1 eq) with 4.7 mg of alpha-Azido-omega-hydroxy 24 (ethylene glycol) (0.0043 mmol, 4 eq) in 0.054 mL H₂O and 0.109 mL DMSO. To the reaction mixture, 0.7 mg Sodium Ascorbate (0.0035 mmol and 4 eq) was added. 0.9 mg Copper Sulfate Pentahydrate (0.0035 mmol, 4 eq) and 1.5 mg tris-hydroxypropyltriazolylmethylamine (0.0035 mmol, 4 eq) were combined in a separate vial, and then added to the reaction mixture. The reaction mixture was stirred overnight at room temperature. LC-MS indicated that the reaction was complete. Compound 74 was purified by dialysis with a Regenerated Cellulose membrane, MWCO: 2 kDa, with solvent changes of 1:1 H₂O/MeOH with 0.01% EDTA (2×), 1:1 H₂O/MeOH (1×), and MeOH (2×). Sample collected and dried on rotovap to obtain 5.1 mg (69.3% yield) of a spectroscopically pure (95.8% AUC at 254 nm) purple solid. MS (ESI) calculated for C₃₉₀H₆₂₅N₃₅O₁₆₂ m/z 8396.4, found 1399.8 (M/6+H)⁺.

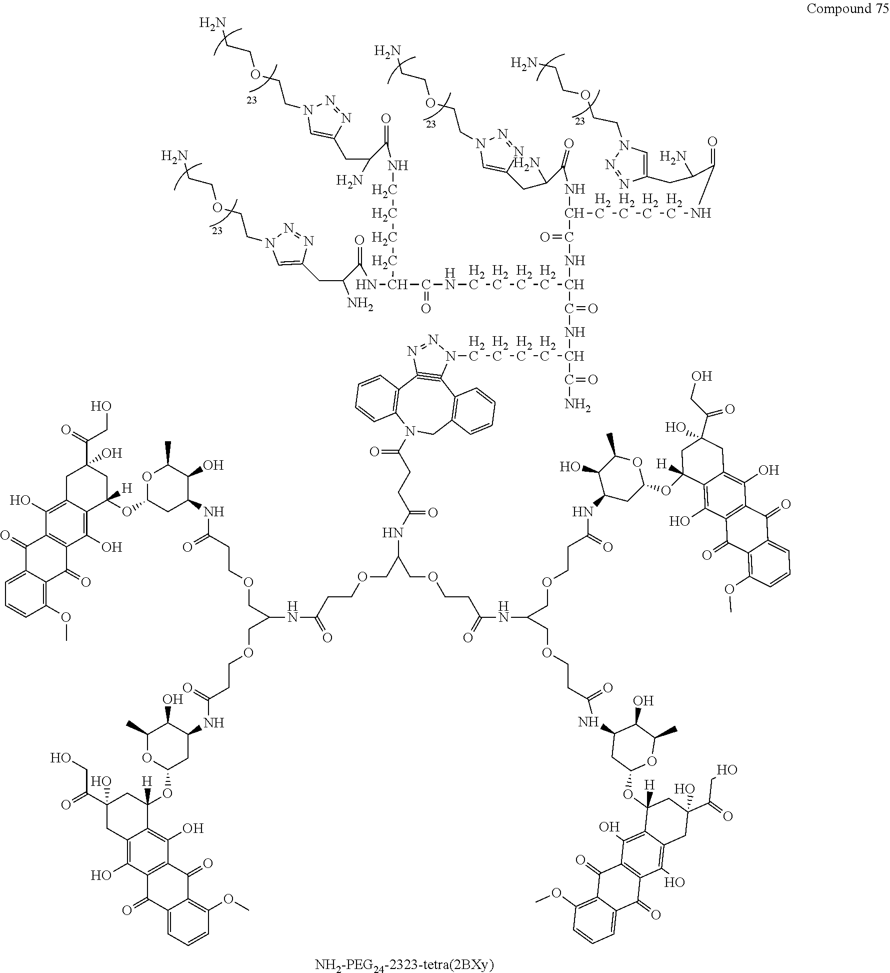

Compound 75, referred to as {NH₂—PEG₂₄}4-{propargyl}₄K2K{Lys(N₃)}-DBCO-2-Amino-1,3-bis(carboxylethoxy)propane(2BXy)₄ or NH₂—PEG₂₄-2323-tetra(2BXy), was synthesized and purified using the same procedure as Compound 74, except alpha-Azido-omega-amino 23 (ethylene glycol) was used as the starting material. Upon collecting purified sample and drying on rotovap, 5.0 mg (68.0% yield) of a spectroscopically impure (67.7% AUC at 254 nm) purple solid was obtained. MS (ESI) calculated for C₃₉₀H₆₂₉N₃₉O₁₅₈ m/z 8387.2, found 1399.2 (M/6+H)⁺.

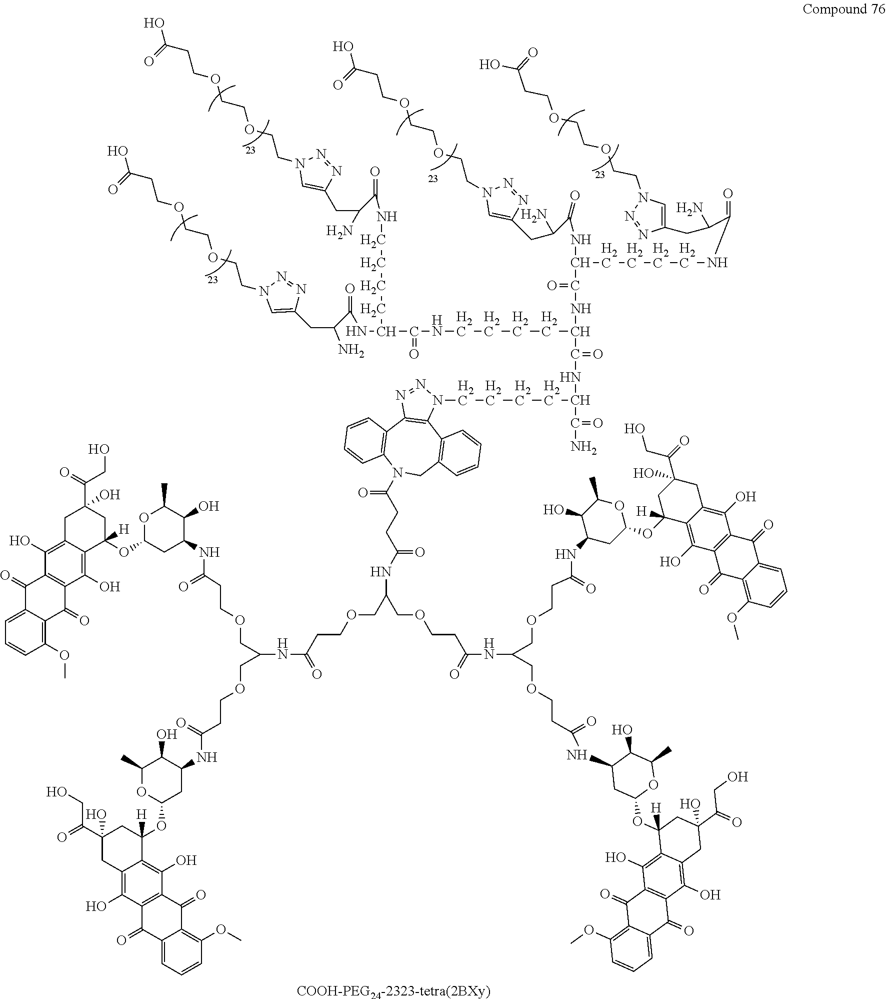

Compound 76, referred to as {COOH-PEG₂₄}4-{propargyl}₄K2K{Lys(N₃)}-DBCO-2-Amino-1,3-bis(carboxylethoxy)propane(2BXy)₄ or COOH-PEG₂₄-2323-tetra(2BXy), was synthesized and purified using the same procedure as Compound 74, except alpha-Azido-omega-(propionic acid) 24 (ethylene glycol) was used as the starting material. Upon collecting purified sample and drying on rotovap, 5.4 mg (71.0% yield) of a spectroscopically pure (90.4% AUC at 254 nm) purple solid was obtained. MS (ESI) calculated for C₄₀₂H₆₄₁N₃₅O₁₇₀ m/z 8679.3, found 1241.2 (M/7+H)⁺.

### Example 11—Synthesis of Additional Amphiphilic Block Copolymers of Formula (S-[B])-H with Brush Architecture

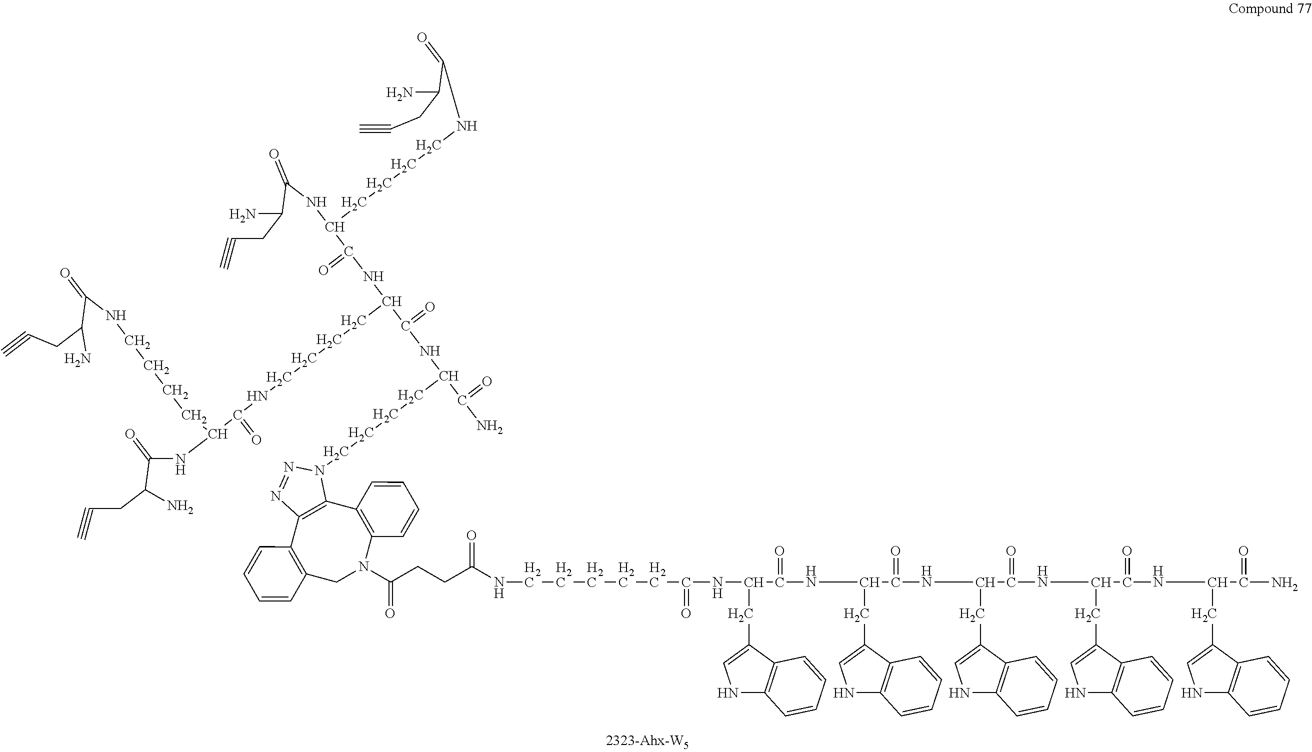 [SMILES: C#CCC(N)C(=O)NCCCCC(NC(=O)C(N)CC#C)C(=O)NCCCCC(NC(=O)C(CCCCNC(=O)C(N)CC#C)NC(=O)C(N)CC#C)C(=O)NC(CCCCn1nnc2c1-c1ccccc1N(C(=O)CCC(=O)NCCCCCC(=O)NC(Cc1c[nH]c3ccccc13)C(=O)NC(Cc1c[nH]c3ccccc13)C(=O)NC(Cc1c[nH]c3ccccc13)C(=O)NC(Cc1c[nH]c3ccccc13)C(=O)NC(Cc1c[nH]c3ccccc13)C(N)=O)Cc1ccccc1-2)C(N)=O]

Compound 77, referred to as {propargyl}₄K2K{Lys(N₃)}-DBCO-Ahx-W₅ or 2323-Ahx-W₅, was synthesized by reacting 28.4 mg of Compound 62 (0.02 mmol, 1.2 eq) dissolved in dry DMSO and 23 mg of {propargyl}4K2K{Lys(N₃)}, which was prepared by solid phase peptide synthesis, (0.024 mmol, 1 eq) as a 100 mg/mL solution in dry DMSO was added. The reaction mixture was stirred overnight at room temperature. HPLC indicated the reaction was complete. Compound 77 was purified on a preparatory HPLC system using a gradient of 25-55% acetonitrile/H₂O (0.05% TFA) over 12 minutes on an Agilent Prep-C18 column, 30×100 mm, 5 μm. The product eluted at ˜4.5 minutes and the resulting fractions were collected, frozen and then lyophilized to obtain 32 mg (80.0% yield) of a spectroscopically pure (99.4% AUC at 254 nm) white powder. MS (ESI) calculated for C₁₂₄H₁₄₆N₂₈O₁₆ m/z 2283.2, found 1142.8 (M/2+H)⁺.

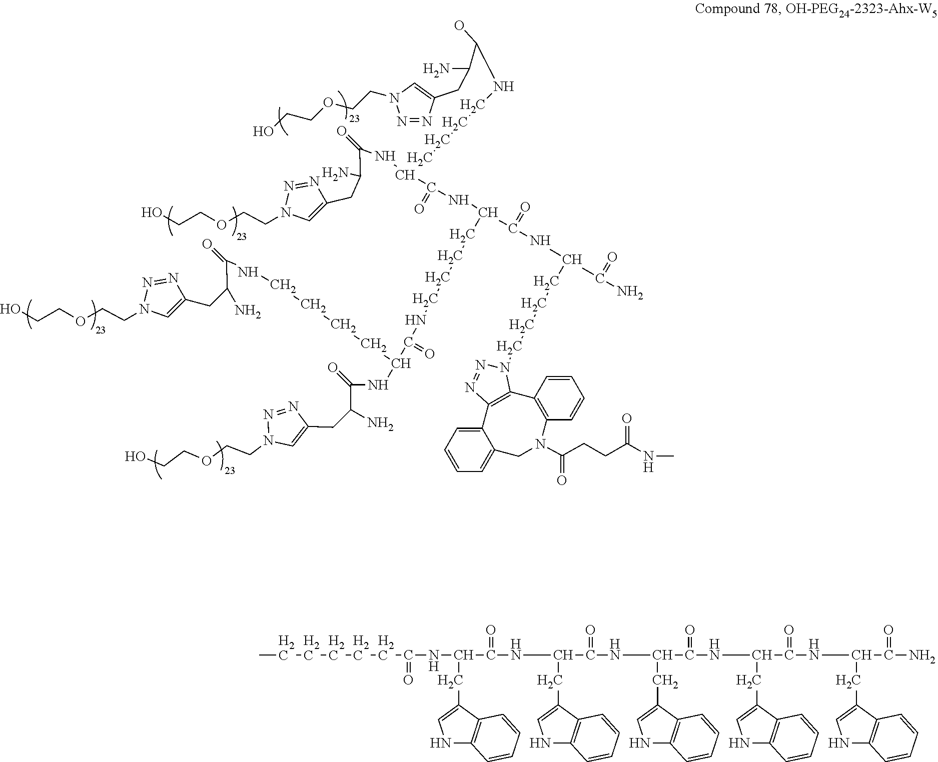

Compound 78, referred to as {OH-PEG2₄}₄-{propargyl}₄K2K{Lys(N₃)}-DBCO-Ahx-W₅ or OH-PEG₂₄-2323-Ahx-W₅, was synthesized by reacting 3.2 mg Compound 73 (0.0014 mmol, 1 eq) with 6.1 mg of alpha-Azido-omega-hydroxy 24 (ethylene glycol) (0.0056 mmol, 4 eq) in 0.063 mL H₂O and 0.126 mL DMSO. To the reaction mixture, 1.1 mg Sodium Ascorbate (0.0056 mmol and 4 eq) was added. 1.4 mg Copper Sulfate Pentahydrate (0.0056 mmol, 4 eq) and 2.4 mg tris-hydroxypropyltriazolylmethylamine (0.0056 mmol, 4 eq) were combined in a separate vial, and then added to the reaction mixture. The reaction mixture was stirred overnight at room temperature. LC-MS indicated that the reaction was complete. Compound 78 was purified by dialysis with a Regenerated Cellulose membrane, MWCO: 2 kDa, with solvent changes of 1:1 H₂O/MeOH with 0.01% EDTA (2×), 1:1 H₂O/MeOH (1×), and MeOH (2×). Sample collected and dried on rotovap to obtain 7.1 mg (75.8% yield) of a spectroscopically impure (63.0% AUC at 254 nm) blue solid. MS (ESI) calculated for C₃₁₆H₅₃₄N₄₀O₁₁₂ m/z 6681.7, found 1337.8 (M/5+H)⁺.

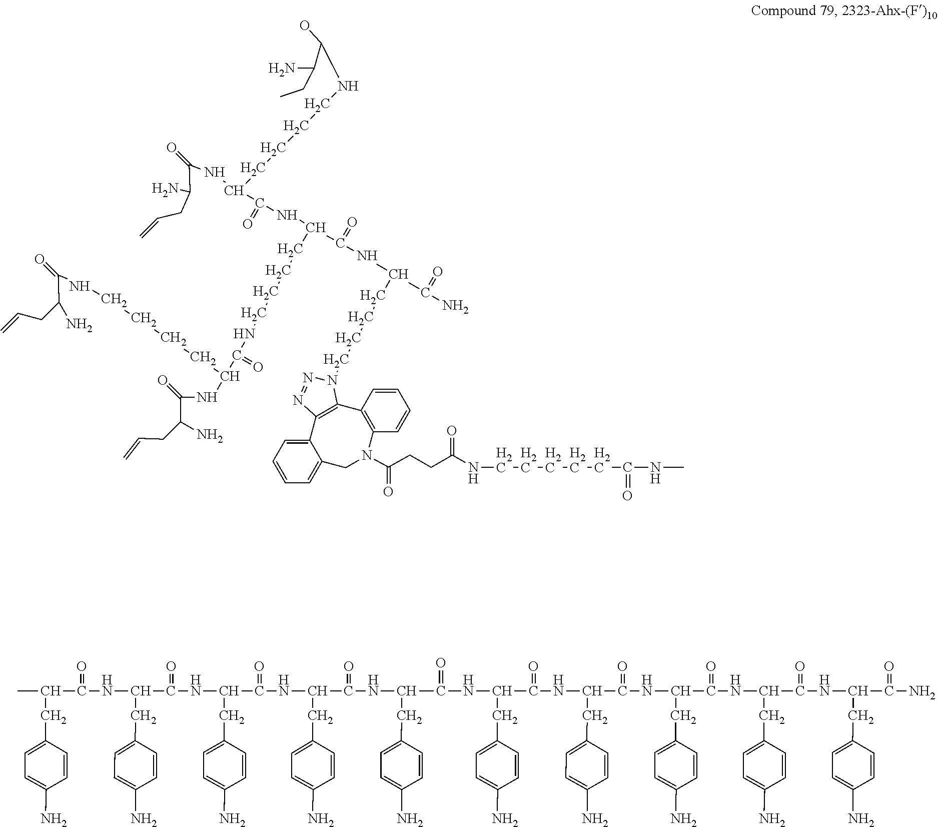

Compound 79, referred to as {propargyl}₄K2K{Lys(N₃)}-DBCO-Ahx-(F′)₁₀ or 2323-Ahx-(F′)₁₀, was synthesized by reacting 88.6 mg of Compound 58 (0.04 mmol, 1.1 eq) and 41 mg of {propargyl}₄K2K{Lys(N₃)}, which was prepared by solid phase peptide synthesis, (0.043 mmol, 1 eq) in 2.0 mL dry DMSO. The reaction mixture was stirred overnight at room temperature. HPLC indicated the reaction was complete and a resulted in a spectroscopically pure (96.1% AUC at 254 nm) colorless solution. MS (ESI) calculated for C₁₅₉H₁₉₆N₃₈O₂₁ m/z 2973.5, found 992.3 (M/3+H)⁺.

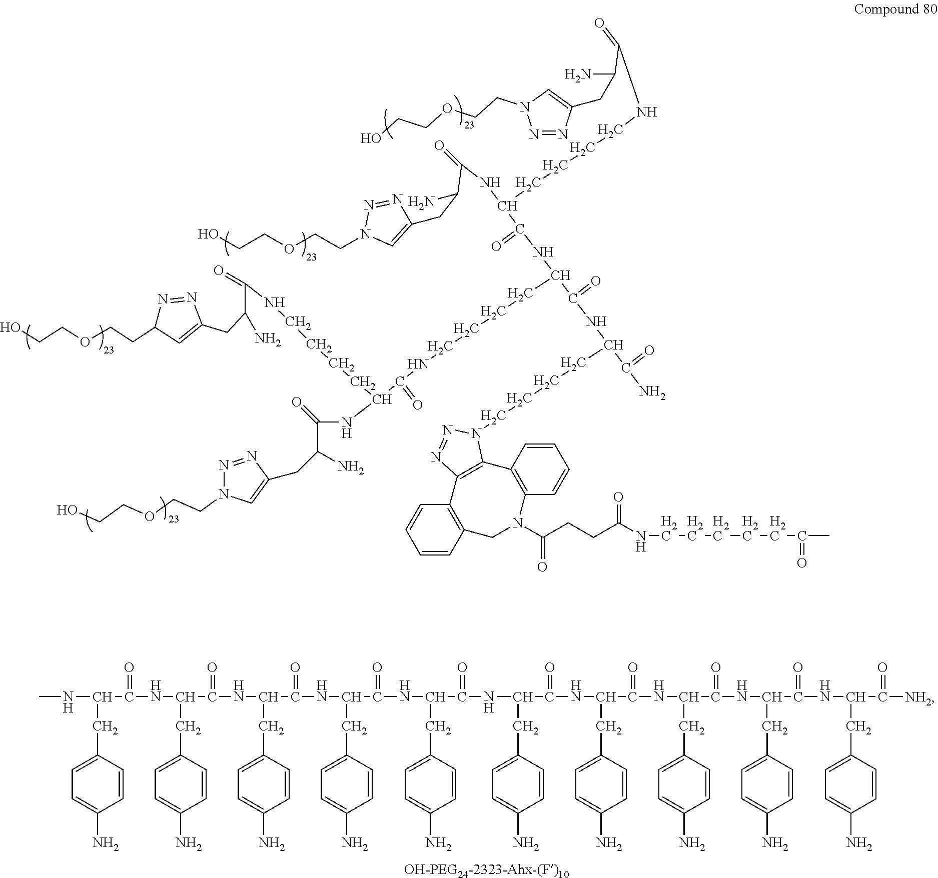

Compound 80, referred to as {OH-PEG₂₄}4-{propargyl}₄K2K{Lys(N₃)}-DBCO-Ahx-(F′)₁₀, or OH-PEG₂₄-2323-Ahx-(F′)₁₀ was synthesized by reacting 4.0 mg Compound 79 (0.0013 mmol, 1 eq) with 5.9 mg of alpha-Azido-omega-hydroxy 24 (ethylene glycol) (0.0054 mmol, 4 eq) in 0.050 mL H₂O and 0.099 mL DMSO. To the reaction mixture, 1.1 mg Sodium Ascorbate (0.0054 mmol, 4 eq) was added. 1.3 mg Copper Sulfate Pentahydrate (0.0054 mmol, 4 eq) and 2.3 mg tris-hydroxypropyltriazolylmethylamine (0.0054 mmol, 4 eq) were combined in a separate vial, and then added to the reaction mixture. The reaction mixture was stirred overnight at room temperature. LC-MS indicated that the reaction was complete. Compound 80 was purified by dialysis with a Regenerated Cellulose membrane, MWCO: 2 kDa, with solvent changes of 1:1 H₂O/MeOH with 0.01% EDTA (2×), 1:1 H₂O/MeOH (1×), and MeOH (2×). Sample collected and dried on rotovap to obtain 6.4 mg (64.6% yield) of a spectroscopically pure (94.1% AUC at 254 nm) green solid. MS (ESI) calculated for C₃₅₁H₅₈₄N₅₀O₁₁₇ m/z 7372.1, found 1230.2 (M/6+H)⁺.

### Example 12—Synthesis of Additional Amphiphilic Block Copolymers of Formula S-[B]-H, and Hydrophobic Polymers of Formula H-D

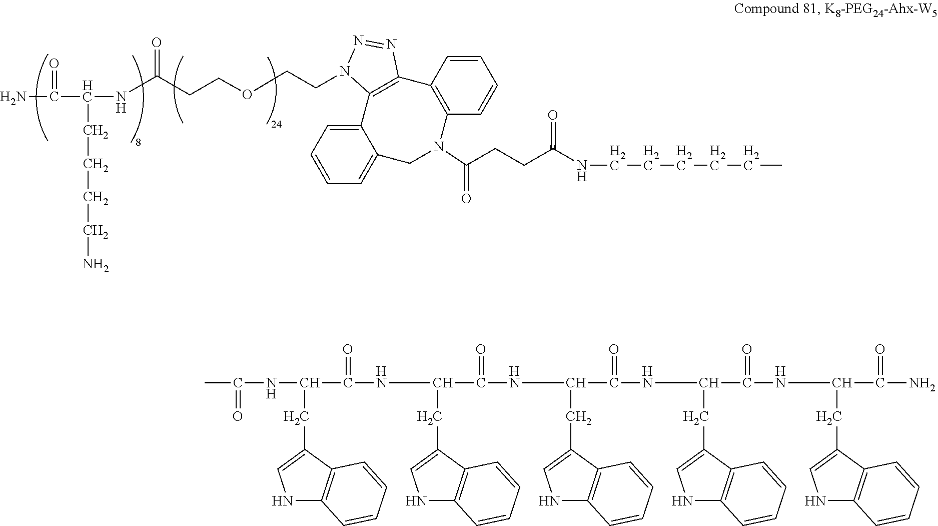

Compound 81, referred to as Ks-PEG₂₄-(N₃-DBCO)-Ahx-W₅ or Ks-PEG₂₄-Ahx-W₅, was synthesized by first suspending K₈-PEG₂₄-N₃, which was prepared by solid phase peptide synthesis, in DMSO at 20 mg/mL and Compound 62 in DMSO at 100 mg/mL and then combining in a reaction vessel at a molar ratio of about 1.1 moles of H for every 1.0 moles of S-B. The reaction was performed at room temperature and determined to be complete after the S-B fragment was fully converted to S-B-H. This resulted in a spectroscopically pure (90.2% AUC at 254 nm) white solution. MS (ESI) calculated for C₁₇₉H₂₇₅N₃₃O₄₁ m/z 3543.0, found 1182.4 (M/3+H)⁺.

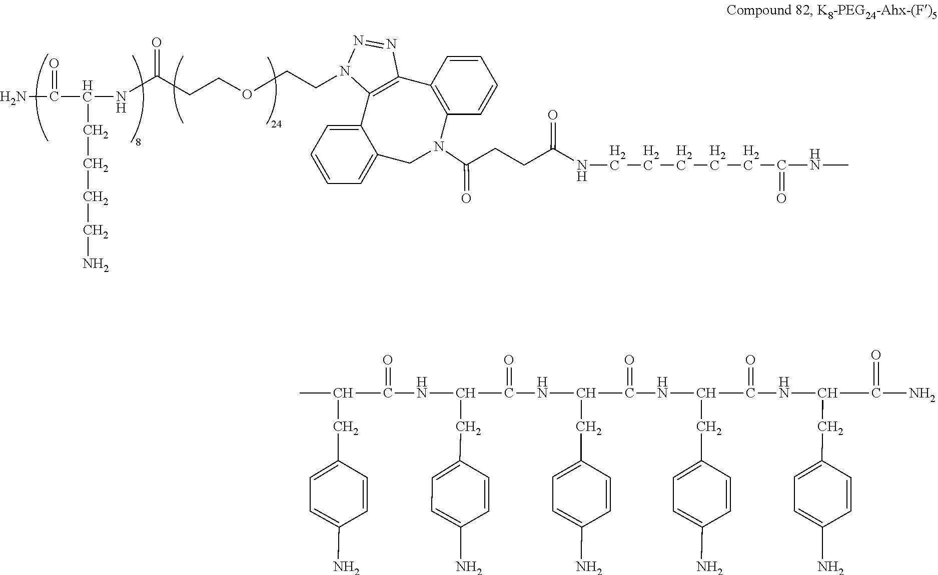

Compound 82, referred to as K₈-PEG₂₄-(N₃-DBCO)-Ahx-(F′)₅ or K₈—PEG₂₄-Ahx-(F′)₅, was synthesized and purified using the same procedure as Compound 81, except Compound 57 was used as the starting material. This resulted in a spectroscopically pure (86.8% AUC at 254 nm) white solution. MS (ESI) calculated for C₁₆₉H₂₇₅N₃₃O₄₁ m/z 3423.0, found 1142.4 (M/3+H)⁺.

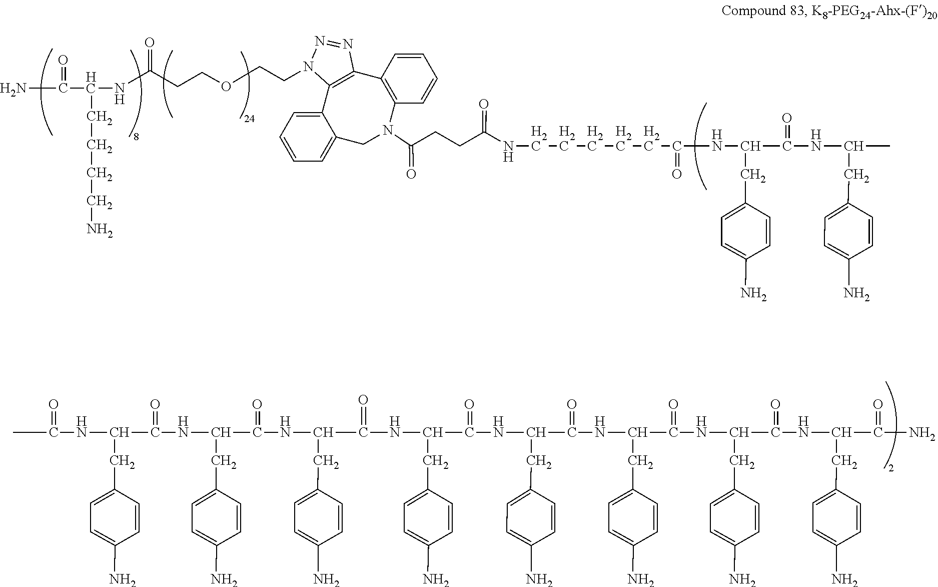

Compound 83, referred to as K₈-PEG₂₄-(N₃-DBCO)-Ahx-(F′)₂₀ or K₈—PEG₂₄-Ahx-(F′)₂0, was synthesized and purified using the same procedure as Compound 81, except Compound 59 was used as the starting material. This resulted in a spectroscopically pure (89.2% AUC at 254 nm) light brown solution. MS (ESI) calculated for C₃₀₄H₄₂₅N₆₃O₅₆ m/z 5854.2, found 1171.2 (M/5+H)⁺.

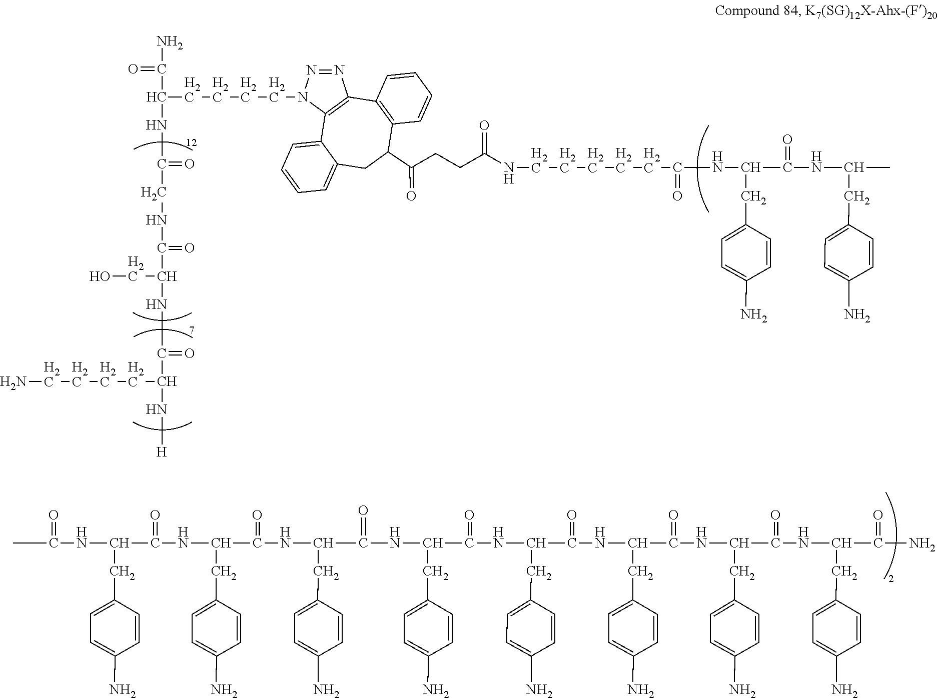

Compound 84, referred to as K₇(SG)₁₂×—(N₃-DBCO)-Ahx-(F′)₂₀ or K₇(SG)₁₂X-Ahx-(F′)₂₀, was synthesized and purified using the same procedure as Compound 81, except K₇(SG)₁₂X-N₃, which was prepared by solid phase peptide synthesis, and Compound 59 were used as the starting materials. This resulted in a spectroscopically pure (76.5% AUC at 254 nm) light brown solution. MS (ESI) calculated for C₃₁₃H₄₂₀N₈₆O₆₇ m/z 6455.2, found 1292.8 (M/5+H)⁺. Note: X=azido-lysine.

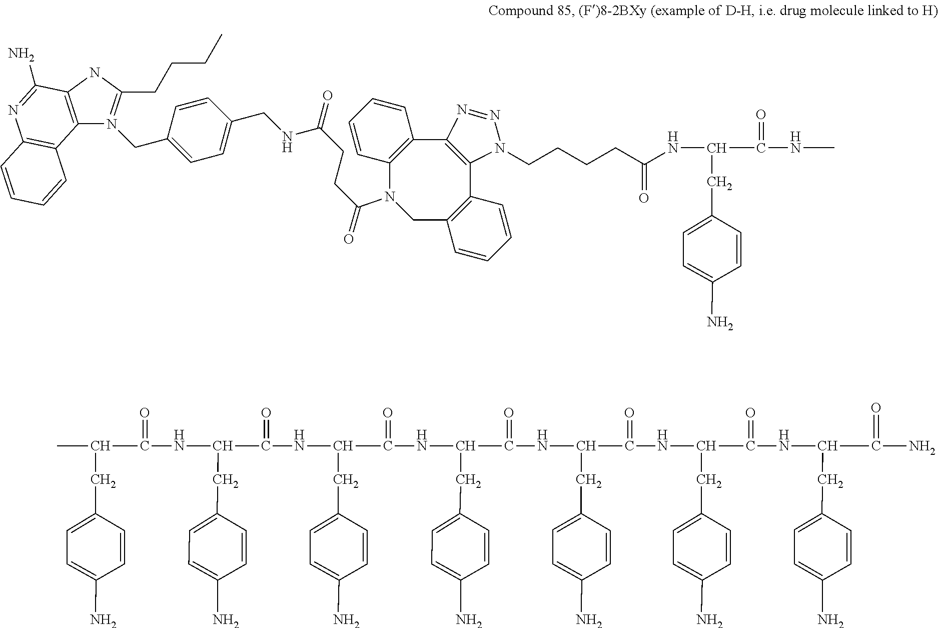

Compound 85, referred to as (F′)₈-(N₃-DBCO)-2BXy or (F′)₈-2BXy, was synthesized and purified using the same procedure as Compound 81, except (F′)₈-N₃, which was prepared by solid phase peptide synthesis, and DBCO-2BXy were used as the starting materials. This resulted in a spectroscopically pure (84.8% AUC at 254 nm) off-white solution. MS (ESI) calculated for C₁₁₈H₁₂₈N₂₆O₁₁ m/z 2085.0, found 1043.6 (M/2+H)⁺. F

### Example 13—Synthesis of S-B-H, Wherein S is a Nucleic Acid-Based Drug Molecule (D) and H Comprises a Poly(Amino Acid)

CpG-based agonists of TLR-9 carry net charge at physiologic pH and can therefore function as the surface stabilizing group (S). To enable site-selective attachment of CpG to hydrophobic polymers or oligomers (S) a CpG sequence comprising an azide was prepared. Briefly, azide-modified CpG ODN 1826 of formula /5AzideN//iSp9/G*G*T*C*C*A*T*G*A*C*G*T*T*C*C*T*G*A*C*G*T*T was custom synthesized by Integrated DNA Technologies (IDT, Coralville, Iowa, USA), wherein /5AzideN//iSp9/ is an azide-terminated PEG3 spacer linked at the 5′-OH of the DNA sequence G*G*T*C*C*A*T*G*A*C*(T*T*C*C*T*G*A*C*G*T*T with a phosphorothioate backbone. As a non-limiting example, DBCO-Ahx-W5 (Compound 62) was reacted with the azide bearing CpG sequence in a DMSO/PBS solution at room temperature to generate an amphiphilic block copolymer, wherein S-B and H were linked together through a triazole group. The reaction was monitored by gel permeation chromatography, which showed that the CpG was completely converted to the product after 16 hours at room temperature. The resulting amphiphilic block copolymer was not turbid (OD at 490 nm<0.04) and formed stable nanoparticle micelles, ˜20 nm, diameter, when resuspended in PBS at 0.5 mg/mL.

### Example 14—Synthesis of S-H, Wherein S is a Hydrophilic Polymer and H Comprises a Poly(Amino Acid)

A pHPMA (HPMA polymer) with a molecular weight of ˜7,000 g/mol terminated with an azide group was prepared as previously described. Briefly, Azide-pHPMA was synthesized via the RAFT polymerization of HPMA using CTA-Azide as a chain transfer agent and ACVA-Azide as an initiator in tert-butanol/N,N-dimethylacetamide at 70° C. for 6.5 hours. The resulting polymer (Azide-pHPMA-DTB) was purified by precipitating in acetone/diethyl ether and dried to yield light pink solid. The CTA (DTB) was removed by reacting the resulting solid in tert-butanol at 80° C. for 2 hours with 20-fold molar excess ACVA. The resulting capped polymer Azide-pHPMA was analyzed by GPC-MALS to confirm that the molecular weight was approximately 7,000 g/mol (or “Daltons” or “Da”), corresponding a polymer with approximately 50 monomeric units. As a non-limiting example, DBCO-Ahx-F′₁₀ (Compound 58) was reacted with azide-pHPMA at room temperature to generate an amphiphilic block copolymer, wherein S and H were linked together through a triazole group. The reaction was monitored by gel permeation chromatography, which showed that the CpG was completely converted to the product after 16 hours at room temperature. The resulting amphiphilic block copolymer was not turbid (OD at 490 nm<0.04) and formed stable nanoparticle micelles, ˜20 nm, diameter, when resuspended in PBS at 0.5 mg/mL.

### Example 15—Combinatorial Synthesis of Amphiphilic Block Copolymers

A combinatorial library of different C-B-H compositions was prepared by reacting different compositions of hydrophobic polymer bearing an alkyne, with different compositions of C-B bearing an azide. Each of the precursors were first suspended in DMSO at greater than 20 mg/mL DMSO, depending on the solubility of the specific composition, sometimes up to 100 mg/mL DMSO, and then combined in a reaction vessel at a molar ratio of about 1.05 moles of H for every 1.0 moles of C-B2. The reactions were performed at room temperature and determined to be complete after the C-B fragment was fully converted to C-B-H.

This reaction scheme was used to prepare different compositions of amphiphilic block copolymers that were characterized for the capacity to form stable nanoparticle micelles in aqueous buffer, PBS pH 7.4, at a concentration of 0.5 mg/mL of amphiphilic block copolymer. The results of these studies are summarized below according to the chemical composition and architecture of the amphiphilic block copolymers.

Linear Peptide

A series of linear amphiphilic block copolymers of formula C-B-H, wherein the charged molecule (C) and spacer (B) comprises peptides, i.e., poly(lysine) and poly(serine-co-glycine), respectively, with varying hydrophobic polymer composition were evaluated for particle size and stability by dynamic light scattering. The results show that nanoparticle micellization is highly dependent on the net charge of these compositions, with C-B-H with net charge of +8 and comprising hydrophobic polymers with up to 20 hydrophobic amino acids based on Phe(NH2), i.e., phenylalanine-amine, sometimes abbreviated F′, forming stable nanoparticle micelles, whereas those with +4 net charge were found to aggregate (Table 3).

TABLE 3
Peptide-based linear amphiphilic block copolymers

| | SEQ | | | Size |
|---|---|---|---|---|
| | ID | Net | | (diameter, |
| Composition (C-B-L-H), L = (Lys(N3-DBCO)) | NO: | charge | MW | nm) |
| KKK-SGSGSGSGSGSGSGSGSGSGSGSG-(Lys(N3-DBCO))-Ahx-(F′)5 | 91 | 4 | 3660.44 | 1013 |
| KKK-SGSGSGSGSGSGSGSGSGSGSGSG-(Lys(N3-DBCO))-Ahx-(F′)10 | 92 | 4 | 4470.64 | 3839 |
| KKK-SGSGSGSGSGSGSGSGSGSGSGSG-(Lys(N3-DBCO))-Ahx-(F′)20 | 93 | 4 | 6092.46 | 1521 |
| KKK-SGSGSGSGSGSGSGSGSGSGSGSG-(Lys(N3-DBCO))-Bis(Ahx-F′10)2 | | 4 | 6419.44 | 2683 |
| KKKKKKK-SGSGSGSGSGSGSGSGSGSGSGSG-(Lys(N3-DBCO))-Ahx-(F′)5 | 95 | 8 | 4173.14 | 477 |
| KKKKKKK-SGSGSGSGSGSGSGSGSGSGSGSG-(Lys(N3-DBCO))-Ahx-(F′)10 | 96 | 8 | 4983.34 | 16 |
| KKKKKKK-SGSGSGSGSGSGSGSGSGSGSGSG-(Lys(N3-DBCO))-Ahx-(F′)20 | 97 | 8 | 6605.16 | 32 |
| KKKKKKK-SGSGSGSGSGSGSGSGSGSGSGSG-(Lys(N3-DBCO))- | | 8 | 6932.14 | 56 |
| (Ahx-(F′)10)2 | | | | |

Note:
single letter abbreviations for amino acids are used in the above table.

Linear PEG

A series of linear amphiphilic block copolymers of formula C-B-H, wherein the charged molecule (C) comprises peptides and the spacer (B) comprises a hydrophilic polymer, i.e. PEG, with varying hydrophobic polymer composition were evaluated for particle size and stability by dynamic light scattering.

Similar to the results observed with amphiphilic block copolymers with peptide-based spacers, nanoparticle micellization was highly dependent on the net charge, with C-B-H with net charge of +8 and comprising hydrophobic polymers with up to 20 hydrophobic amino acids based on F′ forming stable nanoparticle micelles (Table 4). Though, notably, several C-B-H compositions with B comprised of a 24-monomer unit ethylene oxide (PEG) formed stable nanoparticle micelles with as little as +4 net charge, which suggests that spacer groups (B) based on hydrophilic polymers do not require as high charge as those amphiphilic block copolymers with peptide-based spacers.

TABLE 4
Peptide- and hydrophilic polymer-based
linear amphiphilic block copolymers

| | | | Size |
|---|---|---|---|
| Composition (C-B-L-H), L = (Azide- | Net | | (diameter, |
| DBCO) | charge | MW | nm) |
| KK-PEG4-(azide-DBCO)-Ahx-(F′)5 | 2 | 1776.4 | 2427 |
| KK-PEG4-(azide-DBCO)-Ahx-(F′)10 | 2 | 2586.6 | 1112 |
| KK-PEG4-(azide-DBCO)-Ahx-(F′)20 | 2 | 4208.42 | 3618 |
| KK-PEG4-(azide-DBCO)-(Ahx-(F′)10)2 | 2 | 4535.4 | 2650 |
| KK-PEG24-(azide-DBCO)-Ahx-(F′)5 | 2 | 2656.76 | 467 |
| KK-PEG24-(azide-DBCO)-Ahx-(F′)10 | 2 | 3466.96 | 24 |
| KK-PEG24-(azide-DBCO)-Ahx-(F′)20 | 2 | 5088.78 | 2205 |
| KK-PEG24-(azide-DBCO)-(Ahx-(F′)10)2 | 2 | 5415.76 | 1492 |
| KKKK-PEG4-(azide-DBCO)-Ahx-(F′)5 | 4 | 2032.1 | 2069 |
| KKKK-PEG4-(azide-DBCO)-Ahx-(F′)10 | 4 | 2842.3 | 14 |
| KKKK-PEG4-(azide-DBCO)-Ahx-(F′)20 | 4 | 4464.12 | 2890 |
| KKKK-PEG4-(azide-DBCO)-(Ahx- | 4 | 4791.1 | 2121 |
| (F′)10)2 | | | |
| KKKK-PEG24-(azide-DBCO)-Ahx-(F′)5 | 4 | 2913.1 | 1392 |
| KKKK-PEG24-(azide-DBCO)-Ahx-(F′)10 | 4 | 3723.3 | 22 |
| KKKK-PEG24-(azide-DBCO)-Ahx-(F′)20 | 4 | 5345.12 | 103 |
| KKKK-PEG24-(azide-DBCO)-(Ahx- | 4 | 5672.1 | 30 |
| (F′)10)2 | | | |
| KKKKKKKK-PEG4-(azide-DBCO)-Ahx- | 8 | 2544.69 | 1181 |
| (F′)5 | | | |
| KKKKKKKK-PEG4-(azide-DBCO)-Ahx- | 8 | 3354.89 | 11 |
| (F′)10 | | | |
| KKKKKKKK-PEG4-(azide-DBCO)-Ahx- | 8 | 4976.71 | 19 |
| (F′)20 | | | |
| KKKKKKKK-PEG4-(azide-DBCO)- | 8 | 5303.69 | 17 |
| (Ahx-(F′)10)2 | | | |
| KKKKKKKK-PEG24-(azide-DBCO)- | 8 | 3425.79 | 49 |
| Ahx-(F′)5 | | | |
| KKKKKKKK-PEG24-(azide-DBCO)- | 8 | 4235.99 | 16 |
| Ahx-(F′)10 | | | |
| KKKKKKKK-PEG24-(azide-DBCO)- | 8 | 5857.81 | 21 |
| Ahx-(F′)20 | | | |
| KKKKKKKK-PEG24-(azide-DBCO)- | 8 | 6184.79 | 26 |
| (Ahx-(F′)10)2 | | | |

Note:
single letter abbreviations for amino acids are used in the above table; and, oligo(lysine) sequences in the above table were linked to the PEG spacer through the N-terminus and are terminated with an amide.

Dendritic Charged Moiety, with Linear PEG (Cone-Shaped)

A series of cone-shaped amphiphilic block copolymers of formula C-B-L-H, wherein the charged moiety (C) comprises peptides of dendritic structure and the spacer (B) comprises a hydrophilic polymer, i.e. PEG, with varying hydrophobic polymer composition were evaluated for particle size and stability by dynamic light scattering. The cone-shaped structures exhibited overall similar characteristics to amphiphilic block copolymers based on linear C-B-H, wherein B is a hydrophilic polymer, and, notably required up to +8 net charge to stabilize hydrophobic polymers (H) comprised of 20 hydrophobic amino acids based on F (e.g., Ahx-(F′)20) (Table 5).

TABLE 5
Peptide- and hydrophilic polymer-based,
cone-shaped amphiphilic block copolymers

| | | | Size |
|---|---|---|---|
| Composition (C-B-L-H), L = (Lys(N3- | Net | | (diameter, |
| DBCO)) | charge | MW | nm) |
| K2K-PEG4-Lys(N3-DBCO)-Ahx-(F′)5 | 4 | 2033.3 | 1685 |
| K2K-PEG4-Lys(N3-DBCO)-Ahx-(F′)10 | 4 | 2843.5 | 53 |
| K2K-PEG4-Lys(N3-DBCO)-Ahx-(F′)20 | 4 | 4465.32 | 2038 |
| K2K-PEG4-Lys(N3-DBCO)-(Ahx- | 4 | 4792.3 | 2000 |
| (F′)10)2 | | | |
| K2K-PEG24-Lys(N3-DBCO)-Ahx-(F′)5 | 4 | 2913.09 | 3 |
| K2K-PEG24-Lys(N3-DBCO)-Ahx-(F′)10 | 4 | 3723.29 | 24 |
| K2K-PEG24-Lys(N3-DBCO)-Ahx-(F′)20 | 4 | 5345.11 | 5590 |
| K2K-PEG24-Lys(N3-DBCO)-(Ahx- | 4 | 5672.09 | 2000 |
| (F′)10)2 | | | |
| K4K2K-PEG4-Lys(N3-DBCO)-Ahx-(F′)5 | 8 | 2544.7 | 532 |
| K4K2K-PEG4-Lys(N3-DBCO)-Ahx- | 8 | 3354.9 | 10 |
| (F′)10 | | | |
| K4K2K-PEG4-Lys(N3-DBCO)-Ahx- | 8 | 4976.72 | 20 |
| (F′)20 | | | |
| K4K2K-PEG4-Lys(N3-DBCO)-(Ahx- | 8 | 5303.7 | 67 |
| (F′)10)2 | | | |
| K4K2K-PEG24-Lys(N3-DBCO)-Ahx- | 8 | 3425.77 | 892 |
| (F′)5 | | | |
| K4K2K-PEG24-Lys(N3-DBCO)-Ahx- | 8 | 4235.97 | 17 |
| (F′)10 | | | |
| K4K2K-PEG24-Lys(N3-DBCO)-Ahx- | 8 | 5857.79 | 32 |
| (F′)20 | | | |
| K4K2K-PEG24-Lys(N3-DBCO)-(Ahx- | 8 | 6184.77 | 2000 |
| (F′)10)2 | | | |

Note:
single letter abbreviations for amino acids are used in the above table; and, K2K and K4K2K are lysine dendrons comprising 3 and 7 lysines, respectively. For clarity, the structure of K2K (linked to a spacer, B) is shown here for clarity:

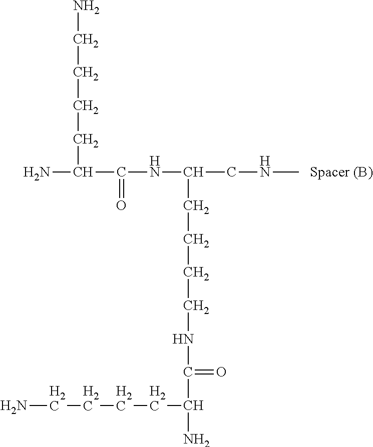 [SMILES: CNCC(CCCCNC(=O)C(N)CCCCN)NC(=O)C(N)CCCCN]

C-B-L-H with Brush Architecture

Finally, a series of brush amphiphilic block copolymers of formula (C-B)y19-K-H, wherein the charged molecule (C) comprises peptides, the spacer (B) comprises a hydrophilic polymer, i.e. PEG, and K is an amplifying linker having 4 sites of attachment (y19=4) for every one hydrophobic polymer, with varying hydrophobic polymer composition were evaluated for particle size and stability by dynamic light scattering.

A striking finding was that the brush amphiphilic block copolymers required less net charge to form stable nanoparticle micelles as compared with the other compositions and architectures of amphiphilic block copolymers. For instance, whereas the linear and cone amphiphilic block copolymers with hydrophobic polymers based on Ahx-(F′)20 and (Ahx-(F′)10)2 with a net charge of +4 were found to form aggregates, indicating insufficient charge stabilization, the brush structures of formula (C-B)y19-K-H all formed stable nanoparticle micelles without presence of aggregates (Table 6).

TABLE 6
Peptide- and hydrophilic polymer-based,
brush-shaped amphiphilic block copolymers

| | | | Size |
|---|---|---|---|
| Composition (C-B-L-H), L = (Lys(N3- | Net | | (diameter, |
| DBCO)) | charge | MW | nm) |
| NH2-PEG24-(azide-propargyl)-4K2K- | 4 | 4234.03 | 8 |
| Lys(N3-DBCO)-Ahx-(F′)10 | | | |
| NH2-PEG24-(azide-propargyl)-4K2K- | 4 | 5855.85 | 14 |
| Lys(N3-DBCO)-Ahx-(F′)20 | | | |
| NH2-PEG24-(azide-propargyl)-4K2K- | 4 | 6182.83 | 11 |
| Lys(N3-DBCO)-(Ahx-(F′)10)2 | | | |
| KK-PEG24-(azide-propargyl)-4K2K- | 8 | 4474.35 | 23 |
| Lys(N3-DBCO)-Ahx-(F′)10 | | | |
| KK-PEG24-(azide-propargyl)-4K2K- | 8 | 6096.17 | 12 |
| Lys(N3-DBCO)-Ahx-(F′)20 | | | |
| KK-PEG24-(azide-propargyl)-4K2K- | 8 | 6423.15 | 14 |
| Lys(N3-DBCO)-(Ahx-(F′)10)2 | | | |

Note:
single letter abbreviations for amino acids are used in the above table; and, oligo(lysine) sequences in the above table were linked to the PEG spacer through the N-terminus and are terminated with an amide.

As shown in Table 7, incorporation of amino acids bearing amines but not carboxylic acids (e.g., glutamic acid) led to improved manufacturability of hydrophobic polymers, e.g., peptide-based hydrophobic polymers based on poly(Trp).

TABLE 7
hydrophobic polymers comprising alkyl amines

| Hydrophobic | SEQ ID | Confirmed | Successful |
|---|---|---|---|
| block precursor | NO: | MW | synthesis |
| Fmoc-WWWWWEWWWW | 99 | — | No |
| Fmoc-WWEWWWWEWW | 100 | — | No |
| Fmoc-EWEWEEWEWE | 101 | 1758.80 | Yes |
| Fmoc-WWWWWKWWWW | 102 | 1821.10 | Yes |
| Fmoc-WWKWWWWKWW | 103 | 1985.30 | Yes |
| Fmoc-KWKWKKWKWK | 104 | 1753.15 | Yes |
| Fmoc-WWWWWKWWWWW | 105 | 2007.30 | Yes |
| Fmoc-KWWKWWKWWKWWKWWK | 106 | 2870.42 | Yes |

Note:
single letter abbreviations for amino acids are used in the above table.

### Example 15—Formulation and Evaluation of Nanoparticle Micelles that Comprise Drug Molecules (D) Based on Different Compositions of S-B-H(D) and S-B-H+D

The above data show that the amphiphilic block copolymers of the present disclosure provide unexpected improvements in manufacturing nanoparticle formulations of drug molecules (D). The next set of studies sough to determine whether or not nanoparticles that comprise amphiphilic block copolymers and immunostimulatory and/or chemotherapeutic drug molecules can mediate durable regression of large, established tumors.

To evaluate a broad range of different formulations based on the amphiphilic block copolymers described herein, the following formulations were prepared:

(a) Nanoparticles comprised of amphiphilic block copolymers that comprise CpG-Ahx-W5 (i.e. CpG oligonucleotide, which itself is an immunostimulatory drug (D), linked to DBCO-Ahx-Trp-Trp-Trp-Trp-Trp through an azide-DBCO linkage, as described under example 13) admixed with Compound 33, an imidazoquinoline-based TLR-7/8a drug (D), referred to as “2BXy,” at a 1 to 4 molar ratio;

(b) Nanoparticles comprised of amphiphilic block copolymers with the formula S-[B]-H that are associated with 2BXy (i.e. S-[B]-H+D) or both 2BXy and Doxorubicin (i.e. S-[B]-H+(1)D+(2)D), including K₈—PEG₂₄-W₅ (Compound 88) admixed with 2BXy at a 1 to 4 molar ratio (i.e. 1 mole of S-B-H to 4 moles of D), K₈—PEG₂₄-F′₂₀ (Compound 82) admixed with 2BXy at a 1 to 4 molar ratio, K₇—SG₁₂-F′₂₀ (Compound 84) admixed with 2BXy at a 1 to 4 molar ratio, HPMA-F′₂₀ (as described under example 13) admixed with 2BXy at a 1 to 4 molar ratio, and [OH-PEG₂₄]4-F′₁₀ (Compound 80) admixed with 2BXy and Doxorubicin at a 1 to 4 to 4 molar ratio, respectively; and,

(c) Nanoparticles comprised of amphiphilic block copolymers with the formula S-[B]-H that have brush S-B architecture and neutral surface charge with a branched poly(amino acid)-based hydrophobic polymer or oligomer (H) that is covalently attached to four 2BXy drug molecules (D), i.e., OH-PEG₂₄]4-2BXy₄ (Compound 80).

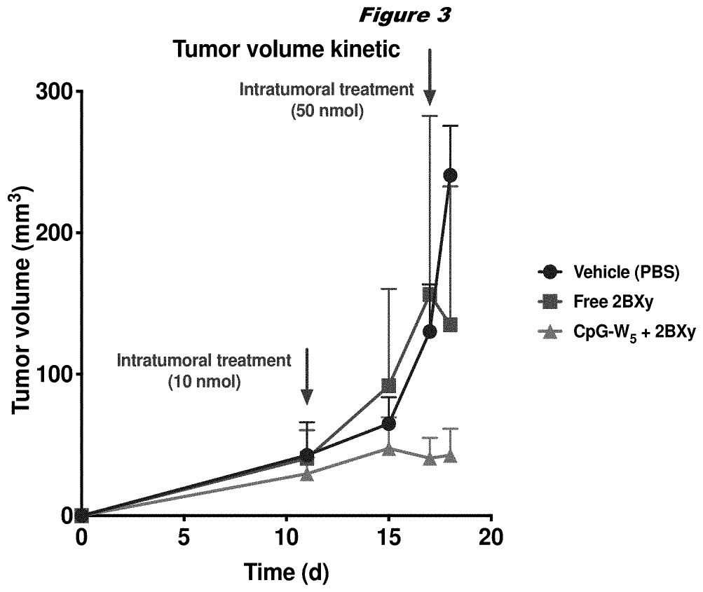

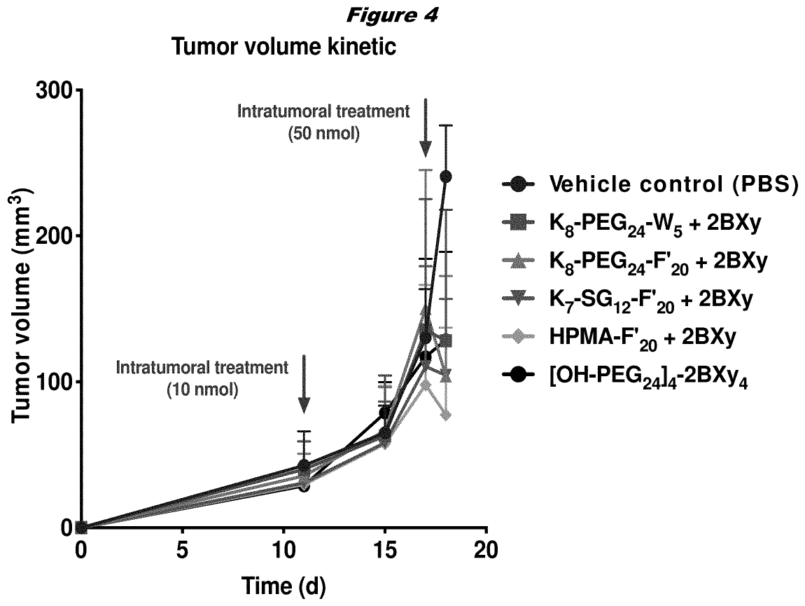

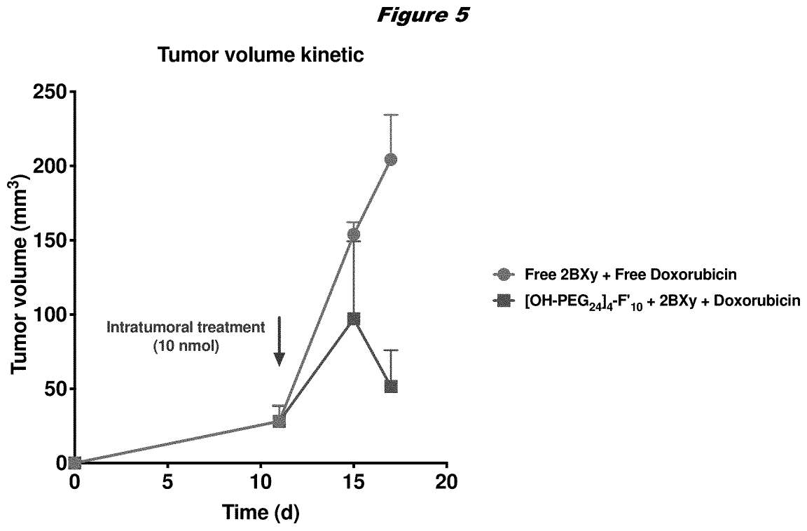

To evaluate the efficacy of the above formulations, C57BL/6 mice were implanted subcutaneously with 1.0×10⁵ MC38 tumor cells and then treated intratumorally (50 μL injection volume) on days 11 and 17 with either PBS alone (‘Naïve’), the soluble drug molecules (2BXy or 2BXy and Doxorubicin) or nanoparticle formulations comprised of an amphiphilic block copolymer with associated or covalently linked drug molecules (S-B-H+D, or S-B-H(D), where D is 2BXy or 2BXy and Doxorubicin). The dose of each active drug was 10 nmol on day 11 and 50 nmol on day 17. Tumors were measured by digital callipers for the longest dimension (‘length’) and the distance perpendicular to the length (‘width’), and volume estimated by the formula: <volume=width×width×length/2>. Data are reported as mean±s.e.m in FIGS. 3-5. Tumor regression is noted as soon as 1 day following 50 nmol dose of each S-B-H carrier of TLR-7/8a.

Importantly, all of the nanoparticle formulations comprised of amphiphilic block copolymers and drug molecules provided enhanced tumor regression as compared with the free drug molecules (FIGS. 3-5). These data indicate that the compositions of amphiphilic block copolymers described herein not only provide unexpected improvements in formulation properties, including defined nanoparticle formulations with high drug loading, but also lead to enhanced in vivo activity for mediating tumor regression using diverse classes of drug molecules, e.g., immunostimulatory and cytotoxic drugs. A non-limiting explanation is that the enhanced drug loading in defined nanoparticle compositions leads to improved drug molecule (D) accumulation in tumors.

Throughout the specification and the claims that follow, unless the context requires otherwise, the words “comprise” and “include” and variations such as “comprising” and “including” will be understood to imply the inclusion of a stated integer or group of integers, but not the exclusion of any other integer or group of integers.

The reference to any prior art in this specification is not, and should not be taken as, an acknowledgement of any form of suggestion that such prior art forms part of the common general knowledge.

It will be appreciated by those skilled in the art that the invention is not restricted in its use to the particular application described. Neither is the present invention restricted in its preferred embodiment with regard to the particular elements and/or features described or depicted herein. It will be appreciated that the invention is not limited to the embodiment or embodiments disclosed, but is capable of numerous rearrangements, modifications and substitutions without departing from the scope of the invention as set forth and defined by the following claims.

Please note that the following claims are provisional claims only, and are provided as examples of possible claims and are not intended to limit the scope of what may be claimed in any future patent applications based on the present application. Integers may be added to or omitted from the example claims at a later date so as to further define or re-define the invention.

1. An amphiphilic block copolymer having any one of the formulas, S-[B]-H, S-[B]-H(D), D-[B]-H, S-B(D)-H, S-[B]-H-[B]-S, S-[B]-H(D)-[B]-S, D-[B]-H-[B]-S, D-[B]-H-[B]-D, S-B(D)-H-[B]-S or S-B(D)-H-B(D)-S; wherein S is a hydrophilic surface stabilizing group; B is a spacer group; H is a hydrophobic polymer or oligomer; D is a drug molecule; ( ) denotes that the group is bonded directly or indirectly as a side chain or as part of a side chain group to the adjacent group; [ ] denotes that the group is optional; and - denotes that each of the adjacent S, B, H or D are linked directly to one another or indirectly to one another via a linker group.

2. The amphiphilic block copolymer according to claim 1, wherein the hydrophobic polymer or oligomer comprises three or more side chain aromatic groups.

3. The amphiphilic block copolymer according to claim 2, wherein the hydrophobic polymer or oligomer comprises three or more side chain aromatic amine groups.

4. The amphiphilic block copolymer according to claim 3, wherein the aromatic amine groups have the formula —Ar—NHR, where Ar is a C6-C10 aromatic group or heterocyclic aromatic group, optionally fused to another ring, and R is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

5. The amphiphilic block copolymer according to any one of claims 1 to 3, wherein the hydrophobic polymer or oligomer is a poly(amino acid) comprising from about 5 to about 50 monomers.

6. The amphiphilic block copolymer according to any one of claims 1 to 4, wherein the hydrophobic polymer or oligomer comprises from about 3 to about 30 aromatic amino acids.

7. The amphiphilic block copolymer according to any one of claims 1 to 6, wherein the hydrophobic polymer or oligomer comprises a poly(amino acid)-based polymer comprised of hydrophobic monomers (e), spacer monomers (m), charged amino acid monomers (n) for charge compensation, and functional group containing monomers (o) for drug molecule (D) attachment.

8. The amphiphilic block copolymer according to any one of claims 1 to 7, wherein the hydrophilic surface stabilizing group comprises one or more charged functional groups.

9. The amphiphilic block copolymer according to claim 8, wherein the surface stabilizing group provides a high net charge (>+4, or <−4).

10. The amphiphilic block copolymer according to any one of claims 1 to 9, wherein the hydrophilic surface stabilizing group comprises one or more mono-saccharide or oligo-saccharide molecules.

11. The amphiphilic block copolymer according to any one of claims 1 to 10, wherein the drug molecule (D) has immunostimulatory properties.

12. The amphiphilic block copolymer according to claim 11, wherein the drug molecule (D) is a PRR agonist.

13. The amphiphilic block copolymer according to claim 12, wherein the drug molecule (D) is a TLR-7 agonist, a TLR-8 agonist and/or a TLR-7/8 agonist.

14. A composition comprising the amphiphilic block copolymer according to any one of claims 1 to 13.

15. A particle comprising the amphiphilic block copolymer according to any one of claims 1 to 13.

16. A polymersome particle comprising the amphiphilic block copolymer according to any one of claims 1 to 13.

17. A micelle particle comprising the amphiphilic block copolymer according to any one of claims 1 to 13.

18. Use of the amphiphilic block copolymer according to any one of claims 1 to 13 to form a particle.

19. Use of the amphiphilic block copolymer according to any one of claims 1 to 13 to form a polymersome particle.

20. Use of the amphiphilic block copolymer according to any one of claims 1 to 13 to form a micelle particle.

21. A mosaic particle comprising two or more different amphiphilic block copolymers selected from any one of the formulas, S-[B]-H, S-[B]-H(D), D-[B]-H, S-B(D)-H, S-[B]-H-[B]-S, S-[B]-H(D)-[B]-S, D-[B]-H-[B]-S, D-[B]-H-[B]-D, S-B(D)-H-[B]-S or S-B(D)-H-B(D)-S; wherein S is a hydrophilic surface stabilizing group; B is a spacer group; H is a hydrophobic polymer or oligomer; D is a drug molecule; ( ) denotes that the group is bonded directly or indirectly as a side chain or as part of a side chain group to the adjacent group; [ ] denotes that the group is optional; and - denotes that each of the adjacent S, B, H or D are linked directly to one another or indirectly to one another via a linker group.

22. A particle of any one of claims 15 to 17 and 21 further comprising a drug molecule hydrophobic polymer or oligomer conjugate, D-H.

23. A method of preparing particles comprising an amphiphilic block copolymer membrane and at least one drug molecule encapsulated therein, said method comprising:
providing an amphiphilic block copolymer having any one of the formulas, S-[B]-H, S-[B]-H(D), D-[B]-H, S-B(D)-H, S-[B]-H-[B]-S, S-[B]-H(D)-[B]-S, D-[B]-H-[B]-S, D-[B]-H-[B]-D, S-B(D)-H-[B]-S or S-B(D)-H-B(D)-S; wherein S is a hydrophilic surface stabilizing group; B is a spacer group; H is a hydrophobic polymer or oligomer; D is a drug molecule; ( ) denotes that the group is bonded directly or indirectly as a side chain or as part of a side chain group to the adjacent group; [ ] denotes that the group is optional; and - denotes that each of the adjacent S, B, H or D are linked directly to one another or indirectly to one another via a linker group; and
preparing an aqueous solution comprising said amphiphilic block copolymer under conditions to produce particles having the at least one drug molecule encapsulated therein.


SEQUENCE LISTING (107 sequences)

SEQ ID NO: 1 - protein, 16 aa, Artificial Sequence
  Synthetic Sequence
DDDDDDDDDDDDDDDD

SEQ ID NO: 2 - protein, 15 aa, Artificial Sequence
  Synthetic Sequence
DDDDDDDDDDDDDDD

SEQ ID NO: 3 - protein, 14 aa, Artificial Sequence
  Synthetic Sequence
DDDDDDDDDDDDDD

SEQ ID NO: 4 - protein, 13 aa, Artificial Sequence
  Synthetic Sequence
DDDDDDDDDDDDD

SEQ ID NO: 5 - protein, 12 aa, Artificial Sequence
  Synthetic Sequence
DDDDDDDDDDDD

SEQ ID NO: 6 - protein, 11 aa, Artificial Sequence
  Synthetic Sequence
DDDDDDDDDDD

SEQ ID NO: 7 - protein, 10 aa, Artificial Sequence
  Synthetic Sequence
DDDDDDDDDD

SEQ ID NO: 8 - protein, 9 aa, Artificial Sequence
  Synthetic Sequence
DDDDDDDDD

SEQ ID NO: 9 - protein, 8 aa, Artificial Sequence
  Synthetic Sequence
DDDDDDDD

SEQ ID NO: 10 - protein, 7 aa, Artificial Sequence
  Synthetic Sequence
DDDDDDD

SEQ ID NO: 11 - protein, 6 aa, Artificial Sequence
  Synthetic Sequence
DDDDDD

SEQ ID NO: 12 - protein, 5 aa, Artificial Sequence
  Synthetic Sequence
DDDDD

SEQ ID NO: 13 - protein, 4 aa, Artificial Sequence
  Synthetic Sequence
DDDD

SEQ ID NO: 14 - protein, 16 aa, Artificial Sequence
  Synthetic Sequence
KKKKKKKKKKKKKKKK

SEQ ID NO: 15 - protein, 15 aa, Artificial Sequence
  Synthetic Sequence
KKKKKKKKKKKKKKK

SEQ ID NO: 16 - protein, 14 aa, Artificial Sequence
  Synthetic Sequence
KKKKKKKKKKKKKK

SEQ ID NO: 17 - protein, 13 aa, Artificial Sequence
  Synthetic Sequence
KKKKKKKKKKKKK

SEQ ID NO: 18 - protein, 12 aa, Artificial Sequence
  Synthetic Sequence
KKKKKKKKKKKK

SEQ ID NO: 19 - protein, 11 aa, Artificial Sequence
  Synthetic Sequence
KKKKKKKKKKK

SEQ ID NO: 20 - protein, 10 aa, Artificial Sequence
  Synthetic Sequence
KKKKKKKKKK

SEQ ID NO: 21 - protein, 9 aa, Artificial Sequence
  Synthetic Sequence
KKKKKKKKK

SEQ ID NO: 22 - protein, 8 aa, Artificial Sequence
  Synthetic Sequence
KKKKKKKK

SEQ ID NO: 23 - protein, 7 aa, Artificial Sequence
  Synthetic Sequence
KKKKKKK

SEQ ID NO: 24 - protein, 6 aa, Artificial Sequence
  Synthetic Sequence
KKKKKK

SEQ ID NO: 25 - protein, 5 aa, Artificial Sequence
  Synthetic Sequence
KKKKK

SEQ ID NO: 26 - protein, 4 aa, Artificial Sequence
  Synthetic Sequence
KKKK

SEQ ID NO: 27 - protein, 4 aa, Artificial Sequence
  Synthetic Sequence
KDKD

SEQ ID NO: 28 - protein, 6 aa, Artificial Sequence
  Synthetic Sequence
KDKDKD

SEQ ID NO: 29 - protein, 4 aa, Artificial Sequence
  Synthetic Sequence
KPLR

SEQ ID NO: 30 - protein, 4 aa, Artificial Sequence
  Synthetic Sequence
YLLL

SEQ ID NO: 31 - protein, 5 aa, Artificial Sequence
  Synthetic Sequence
LLLLL

SEQ ID NO: 32 - protein, 10 aa, Artificial Sequence
  Synthetic Sequence
LLLLLLLLLL

SEQ ID NO: 33 - protein, 5 aa, Artificial Sequence
  Synthetic Sequence
FFFFF

SEQ ID NO: 34 - protein, 10 aa, Artificial Sequence
  Synthetic Sequence
FFFFFFFFFF

SEQ ID NO: 35 - protein, 5 aa, Artificial Sequence
  Synthetic Sequence
HHHHH

SEQ ID NO: 36 - protein, 10 aa, Artificial Sequence
  Synthetic Sequence
HHHHHHHHHH

SEQ ID NO: 37 - protein, 5 aa, Artificial Sequence
  Synthetic Sequence
WWWWW

SEQ ID NO: 38 - protein, 10 aa, Artificial Sequence
  Synthetic Sequence
WWWWWWWWWW

SEQ ID NO: 39 - protein, 5 aa, Artificial Sequence
  Synthetic Sequence
  misc_feature (1)..(5): Xaa is a para-amino-phenylalanine
XXXXX

SEQ ID NO: 40 - protein, 10 aa, Artificial Sequence
  Synthetic Sequence
  misc_feature (1)..(10): Xaa is a para-amino-phenylalanine
XXXXXXXXXX

SEQ ID NO: 41 - protein, 20 aa, Artificial Sequence
  Synthetic Sequence
  misc_feature (1)..(20): Xaa is a para-amino-phenylalanine
XXXXXXXXXXXXXXXXXXXX

SEQ ID NO: 42 - protein, 30 aa, Artificial Sequence
  Synthetic Sequence
  misc_feature (1)..(30): Xaa is a para-amino-phenylalanine
XXXXXXXXXXXXXXXXXXXXXXXXXXXXXX

SEQ ID NO: 43 - protein, 10 aa, Artificial Sequence
  Synthetic Sequence
FWFWFWFWFW

SEQ ID NO: 44 - protein, 20 aa, Artificial Sequence
  Synthetic Sequence
  misc_feature (2)..(2): Xaa is a para-amino-phenylalanine
  misc_feature (4)..(4): Xaa is a para-amino-phenylalanine
  misc_feature (6)..(6): Xaa is a para-amino-phenylalanine
  misc_feature (8)..(8): Xaa is a para-amino-phenylalanine
  misc_feature (10)..(10): Xaa is a para-amino-phenylalanine
  misc_feature (12)..(12): Xaa is a para-amino-phenylalanine
  misc_feature (14)..(14): Xaa is a para-amino-phenylalanine
  misc_feature (16)..(16): Xaa is a para-amino-phenylalanine
  misc_feature (18)..(18): Xaa is a para-amino-phenylalanine
  misc_feature (20)..(20): Xaa is a para-amino-phenylalanine
WXWXWXWXWXWXWXWXWXWX

SEQ ID NO: 45 - protein, 5 aa, Artificial Sequence
  Synthetic Sequence
  MISC_FEATURE (1)..(1): Wherein Phe comprises a DBCO linkage.
FFFFF

SEQ ID NO: 46 - protein, 5 aa, Artificial Sequence
  Synthetic Sequence
  MISC_FEATURE (1)..(1): Wherein Trp comprises a DBCO linkage.
WWWWW

SEQ ID NO: 47 - protein, 5 aa, Artificial Sequence
  Synthetic Sequence
  misc_feature (1)..(5): Xaa is a para-amino-phenylalanine
  misc_feature (1)..(1): Wherein Xaa is comprises a DBCO linkage.
XXXXX

SEQ ID NO: 48 - protein, 10 aa, Artificial Sequence
  Synthetic Sequence
  misc_feature (1)..(10): Xaa is a para-amino-phenylalanine
  misc_feature (1)..(1): Wherein Xaa is comprises with a DBCO linkage.
XXXXXXXXXX

SEQ ID NO: 49 - protein, 20 aa, Artificial Sequence
  Synthetic Sequence
  misc_feature (1)..(20): Xaa is a para-amino-phenylalanine
  misc_feature (1)..(1): Wherein Phe comprises a DBCO linkage.
XXXXXXXXXXXXXXXXXXXX

SEQ ID NO: 50 - protein, 7 aa, Artificial Sequence
  Synthetic Sequence
  MISC_FEATURE (7)..(7): Xaa is a azidolysine (Lys(N3))
KKSLVRX

SEQ ID NO: 51 - protein, 13 aa, Artificial Sequence
  Synthetic Sequence
  misc_feature (8)..(8): Xaa is a azidolysine (Lys(N3))
  misc_feature (8)..(8): Wherein Xaa is connected to Phe via a N3-DBCO linkage
KKKSLVRXFFFFF

SEQ ID NO: 52 - protein, 10 aa, Artificial Sequence
  Synthetic Sequence
  MISC_FEATURE (5)..(5): Wherein Glu is connected to Phe via a N3-DBCO linkage.
EEEEEFFFFF

SEQ ID NO: 53 - protein, 13 aa, Artificial Sequence
  Synthetic Sequence
  misc_feature (8)..(8): Xaa is a azidolysine (Lys(N3))
  misc_feature (8)..(8): Wherein Xaa is connected to Trp via a N3-DBCO linkage.
KKKSLVRXWWWWW

SEQ ID NO: 54 - protein, 10 aa, Artificial Sequence
  Synthetic Sequence
  MISC_FEATURE (5)..(5): Wherein Glu is connected to Trp via a N3-DBCO linkage.
EEEEEWWWWW

SEQ ID NO: 55 - protein, 13 aa, Artificial Sequence
  Synthetic Sequence
  misc_feature (8)..(8): Xaa is a azidolysine (Lys(N3))
  misc_feature (9)..(13): Xaa is a para-amino-phenylalanine
  misc_feature (8)..(8): Wherein Xaa is connected to Xaa via a N3-DBCO linkage.
KKKSLVRXXXXXX

SEQ ID NO: 56 - protein, 10 aa, Artificial Sequence
  Synthetic Sequence
  misc_feature (5)..(5): Wherein Glu is connected to Xaa via a N3-DBCO linkage.
  misc_feature (6)..(10): Xaa is a para-amino-phenylalanine
EEEEEXXXXX

SEQ ID NO: 57 - protein, 18 aa, Artificial Sequence
  Synthetic Sequence
  misc_feature (8)..(8): Xaa is a azidolysine (Lys(N3))
  misc_feature (9)..(18): Xaa is a para-amino-phenylalanine
  misc_feature (8)..(8): Wherein Xaa is connected to Xaa via a N3-DBCO linkage.
KKKSLVRXXXXXXXXXXX

SEQ ID NO: 58 - protein, 15 aa, Artificial Sequence
  Synthetic Sequence
  misc_feature (5)..(5): Wherein Glu is connected to Xaa via a N3-DBCO linkage
  misc_feature (6)..(15): Xaa is a para-amino-phenylalanine
EEEEEXXXXXXXXXX

SEQ ID NO: 59 - protein, 10 aa, Artificial Sequence
  Synthetic Sequence
  misc_feature (1)..(10): Xaa is a para-amino-phenylalanine
  misc_feature (1)..(1): Wherein Xaa comprises Hydroxyl-PEG24-N3-DBCO linked to the N-terminus
XXXXXXXXXX

SEQ ID NO: 60 - protein, 17 aa, Artificial Sequence
  Synthetic Sequence
  misc_feature (7)..(7): Wherein Asp comprises PEG24-N3-DBCO linked at the C-terminus
  misc_feature (8)..(17): Xaa is a para-amino-phenylalanine
  misc_feature (8)..(8): Wherein Xaa is connected to Asp via a PEG24-N3- DBCO linkage.
DDDDDDDXXXXXXXXXX

SEQ ID NO: 61 - protein, 21 aa, Artificial Sequence
  Synthetic Sequence
  misc_feature (11)..(11): Xaa is a azidolysine (Lys(N3))
  misc_feature (12)..(21): Xaa is a para-amino-phenylalanine
  misc_feature (11)..(11): Wherein Xaa is connected to Xaa via a N3-DBCO linkage
KKKKKKKKKKXXXXXXXXXXX

SEQ ID NO: 62 - protein, 28 aa, Artificial Sequence
  Synthetic Sequence
  misc_feature (8)..(8): Xaa is a azidolysine (Lys(N3))
  misc_feature (9)..(28): Xaa is a para-amino-phenylalanine
  misc_feature (8)..(8): Wherein Xaa is connected to Xaa via a N3-DBCO linkage
KKKSLVRXXXXXXXXXXXXXXXXXXXXX

SEQ ID NO: 63 - protein, 25 aa, Artificial Sequence
  Synthetic Sequence
  misc_feature (5)..(5): Wherein Glu is connected to Xaa via a N3-DBCO linkage
  misc_feature (6)..(25): Xaa is para-amino-phenylalanine
EEEEEXXXXXXXXXXXXXXXXXXXX

SEQ ID NO: 64 - protein, 5 aa, Artificial Sequence
  Synthetic Sequence
  MISC_FEATURE (1)..(1): Wherein Glu is modified with an Fmoc group at the N-terminus
  MISC_FEATURE (5)..(5): Wherein Glu is modified with an amide (NH2) at the C-terminus
EEEEE

SEQ ID NO: 65 - protein, 5 aa, Artificial Sequence
  Synthetic Sequence
  MISC_FEATURE (1)..(1): Wherein Glu is modified with an Fmoc group at the N-terminus
  MISC_FEATURE (5)..(5): Wherein Glu is modified with an amide (NH2) at the C-terminus
EWEWE

SEQ ID NO: 66 - protein, 5 aa, Artificial Sequence
  Synthetic Sequence
  MISC_FEATURE (1)..(1): Wherein Trp is modified with an Fmoc group at the N-terminus
  MISC_FEATURE (5)..(5): Wherein Trp is modified with an amide (NH2) at the C-terminus
WEWEW

SEQ ID NO: 67 - protein, 10 aa, Artificial Sequence
  Synthetic Sequence
  MISC_FEATURE (1)..(1): Wherein Trp is modified with an Fmoc group at the N-terminus
  MISC_FEATURE (10)..(10): Wherein Trp is modified with an amide (NH2) at the C-terminus
WWEWWWWEWW

SEQ ID NO: 68 - protein, 5 aa, Artificial Sequence
  Synthetic Sequence
  MISC_FEATURE (1)..(1): Wherein Trp is modified with an Fmoc group at the N-terminus
  MISC_FEATURE (5)..(5): Wherein Trp is modified with an amide (NH2) at the C-terminus
WWEWW

SEQ ID NO: 69 - protein, 5 aa, Artificial Sequence
  Synthetic Sequence
  MISC_FEATURE (1)..(1): Wherein Glu is modified with an Fmoc group at the N-terminus
  MISC_FEATURE (5)..(5): Wherein Glu is modified with an amide (NH2) at the C-terminus
EWEWE

SEQ ID NO: 70 - protein, 10 aa, Artificial Sequence
  Synthetic Sequence
  MISC_FEATURE (1)..(1): Wherein Glu is modified with an Fmoc group at the N-terminus
  MISC_FEATURE (10)..(10): Wherein Glu is modified with an amide (NH2) at the C-terminus
EWEWEEWEWE

SEQ ID NO: 71 - protein, 10 aa, Artificial Sequence
  Synthetic Sequence
  MISC_FEATURE (1)..(1): Wherein Trp is modified with an Fmoc group at the N-terminus
  MISC_FEATURE (10)..(10): Wherein Trp is modified with an amide (NH2) at the C-terminus
WEWEWWEWEW

SEQ ID NO: 72-75 - skipped (4 entries)

SEQ ID NO: 76 - protein, 15 aa, Artificial Sequence
  Synthetic Sequence
  MISC_FEATURE (1)..(1): Wherein Gly is modified with an Fmoc group at the N-terminus
  MISC_FEATURE (15)..(15): Wherein Glu is modified with an amide (NH2) at the C-terminus
GGEGGWGGEGGWGGE

SEQ ID NO: 77 - protein, 15 aa, Artificial Sequence
  Synthetic Sequence
  MISC_FEATURE (1)..(1): Wherein Gly is modified with an amide (NH2) at the N-terminus
  MISC_FEATURE (15)..(15): Wherein Glu is modified with an amide (NH2) at the C-terminus
GGEGGWGGEGGWGGE

SEQ ID NO: 78 - protein, 15 aa, Artificial Sequence
  Synthetic Sequence
  MISC_FEATURE (1)..(1): Wherein Gly is linked to DBCO linkage
  MISC_FEATURE (15)..(15): Wherein Glu is modified with an amide (NH2) at the C-terminus
GGEGGWGGEGGWGGE

SEQ ID NO: 79 - skipped

SEQ ID NO: 80 - protein, 30 aa, Artificial Sequence
  Synthetic Sequence
  MISC_FEATURE (1)..(1): Wherein Lys is modified with an Fmoc group at the N-terminus
  MISC_FEATURE (30)..(30): Wherein Gly is modified with an amide (NH2) at the C-terminus
KGWGWGKGWGWGKGWGWGKGWGWGKGWGWG

SEQ ID NO: 81 - protein, 10 aa, Artificial Sequence
  Synthetic Sequence
  MISC_FEATURE (1)..(1): Wherein Glu is modified with an Azido group at the N-terminus
  MISC_FEATURE (10)..(10): Wherein Glu is modified with an amide (NH2) at the C-terminus
EEEEEEEEEE

SEQ ID NO: 82 - protein, 10 aa, Artificial Sequence
  Synthetic Sequence
  MISC_FEATURE (1)..(1): Wherein Lys is modified with an Azido group at the N-terminus
  MISC_FEATURE (10)..(10): Wherein Lys is modified with an amide (NH2) at the C-terminus
KKKKKKKKKK

SEQ ID NO: 83 - protein, 10 aa, Artificial Sequence
  Synthetic Sequence
  MISC_FEATURE (1)..(1): Wherein Xaa is linked to a Ahx
  MISC_FEATURE (20)..(20): Wherein Xaa is modified with an amide (NH2) at the C-terminus
  misc_feature (1)..(10): Xaa is a para-amino-phenylalanine
XXXXXXXXXX

SEQ ID NO: 84 - protein, 20 aa, Artificial Sequence
  Synthetic Sequence
  MISC_FEATURE (1)..(1): Wherein Xaa is linked to a DBCO-Ahx
  MISC_FEATURE (20)..(20): Wherein Xaa is modified with an amide (NH2) at the C-terminus
  misc_feature (1)..(20): Xaa is a para-amino-phenylalanine
XXXXXXXXXXXXXXXXXXXX

SEQ ID NO: 85 - protein, 20 aa, Artificial Sequence
  Synthetic Sequence
  MISC_FEATURE (1)..(1): Wherein Xaa is linked to a Ahx
  MISC_FEATURE (20)..(20): Wherein Xaa is modified with an amide (NH2) at the C-terminus
  misc_feature (1)..(20): Xaa is a para-amino-phenylalanine
XXXXXXXXXXXXXXXXXXXX

SEQ ID NO: 86 - skipped

SEQ ID NO: 87 - protein, 10 aa, Artificial Sequence
  Synthetic Sequence
  MISC_FEATURE (1)..(1): Wherein Xaa is linked to a Ahx
  MISC_FEATURE (10)..(10): Wherein Xaa is modified with an amide (NH2) at the C-terminus
  misc_feature (1)..(10): Xaa ia para-amino-phenylalanine
XXXXXXXXXX

SEQ ID NO: 88 - protein, 5 aa, Artificial Sequence
  Synthetic Sequence
  MISC_FEATURE (1)..(1): Wherein Trp is linked to a Ahx
  MISC_FEATURE (5)..(5): Wherein Xaa is modified with an amide (NH2) at the C-terminus
WWWWW

SEQ ID NO: 89-90 - skipped (2 entries)

SEQ ID NO: 91 - protein, 33 aa, Artificial Sequence
  Synthetic Sequence
  misc_feature (28)..(28): Wherein Lys is connected to Xaa via a N3-DBCO- Ahx.
  misc_feature (29)..(33): Xaa is a para-amino-phenylalanine
KKKSGSGSGSGSGSGSGSGSGSGSGSGKXXXXX

SEQ ID NO: 92 - protein, 38 aa, Artificial Sequence
  Synthetic Sequence
  misc_feature (28)..(28): Wherein Lys is connected to Xaa via a N3-DBCO- Ahx.
  misc_feature (29)..(38): Xaa is a para-amino-phenylalanine
KKKSGSGSGSGSGSGSGSGSGSGSGSGKXXXXXXXXXX

SEQ ID NO: 93 - protein, 48 aa, Artificial Sequence
  Synthetic Sequence
  misc_feature (28)..(28): Wherein Lys is connected to Xaa via a N3-DBCO- Ahx.
  misc_feature (29)..(48): Xaa is a para-amino-phenylalanine
KKKSGSGSGSGSGSGSGSGSGSGSGSGKXXXXXXXXXXXXXXXXXXXX

SEQ ID NO: 94 - skipped

SEQ ID NO: 95 - protein, 37 aa, Artificial Sequence
  Synthetic Sequence
  misc_feature (32)..(32): Wherein Lys is connected to Xaa via a N3-DBCO- Ahx.
  misc_feature (33)..(37): Xaa is a para-amino-phenylalanine
KKKKKKKSGSGSGSGSGSGSGSGSGSGSGSGKXXXXX

SEQ ID NO: 96 - protein, 42 aa, Artificial Sequence
  Synthetic Sequence
  misc_feature (32)..(32): Wherein Lys is connected to Xaa via a N3-DBCO- Ahx.
  misc_feature (33)..(42): Xaa is a para-amino-phenylalanine
KKKKKKKSGSGSGSGSGSGSGSGSGSGSGSGKXXXXXXXXXX

SEQ ID NO: 97 - protein, 52 aa, Artificial Sequence
  Synthetic Sequence
  misc_feature (32)..(32): Wherein Lys is connected to Xaa via a N3-DBCO- Ahx.
  misc_feature (33)..(52): Xaa is a para-amino-phenylalanine
KKKKKKKSGSGSGSGSGSGSGSGSGSGSGSGKXXXXXXXXXXXXXXXXXXXX

SEQ ID NO: 98 - skipped

SEQ ID NO: 99 - protein, 10 aa, Artificial Sequence
  Synthetic Sequence
  MISC_FEATURE (1)..(1): Wherein Trp is modified with an Fmoc group at the N-terminus
WWWWWEWWWW

SEQ ID NO: 100 - protein, 10 aa, Artificial Sequence
  Synthetic Sequence
  MISC_FEATURE (1)..(1): Wherein Trp is modified with an Fmoc group at the N-terminus
WWEWWWWEWW

SEQ ID NO: 101 - protein, 10 aa, Artificial Sequence
  Synthetic Sequence
  MISC_FEATURE (1)..(1): Wherein Glu is modified with an Fmoc group at the N-terminus
EWEWEEWEWE

SEQ ID NO: 102 - protein, 10 aa, Artificial Sequence
  Synthetic Sequence
  MISC_FEATURE (1)..(1): Wherein Trp is modified with an Fmoc group at the N-terminus
WWWWWKWWWW

SEQ ID NO: 103 - protein, 10 aa, Artificial Sequence
  Synthetic Sequence
  MISC_FEATURE (1)..(1): Wherein Trp is modified with an Fmoc group at the N-terminus
WWKWWWWKWW

SEQ ID NO: 104 - protein, 10 aa, Artificial Sequence
  Synthetic Sequence
  MISC_FEATURE (1)..(1): Wherein Lys is modified with an Fmoc group at the N-terminus
KWKWKKWKWK

SEQ ID NO: 105 - protein, 11 aa, Artificial Sequence
  Synthetic Sequence
  MISC_FEATURE (1)..(1): Wherein Trp is modified with an Fmoc group at the N-terminus
WWWWWKWWWWW

SEQ ID NO: 106 - protein, 16 aa, Artificial Sequence
  Synthetic Sequence
  MISC_FEATURE (1)..(1): Wherein Lys is modified with an Fmoc group at the N-terminus
KWWKWWKWWKWWKWWK

SEQ ID NO: 107 - protein, 4 aa, Artificial Sequence
  Synthetic Sequence
GFLG